(19) United States
(12) Patent Application Publication
(10) Pub. No.: US 2026/0000743 A1
(43) Pub. Date: Jan. 1, 2026

(54) IMMUNOGENIC COMPOSITIONS COMPRISING NUCLEIC ACIDS FOR RAS PEPTIDES

(71) Applicant: Think Therapeutics, Inc., Newton, MA (US)

(72) Inventors: David Kenneth GIFFORD, Newton, MA (US); Brandon CARTER, Cambridge, MA (US)

(21) Appl. No.: 19/236,476

(22) Filed: Jun. 12, 2025

Related U.S. Application Data
(63) Continuation of application No. 17/815,086, filed on Jul. 26, 2022, now Pat. No. 12,364,741.
Application No. 17/815,086 is a division of application No. 17/551,679, filed on Dec. 15, 2021, now Pat. No. 11,464,837.
Application No. 17/551,679 is a continuation of application No. 17/336,960, filed on Jun. 2, 2021, now Pat. No. 11,235,039.
Application No. 17/336,960 is a continuation of application No. 17/100,630, filed on Nov. 20, 2020, now Pat. No. 11,058,751.

(51) Int. Cl. A61K 39/00; A61P 35/00

(52) U.S. Cl. CPC A61K 39/0005; A61K 39/0011; A61P 35/00; C12Y 306/05002; A61K 2039/585

(57) ABSTRACT

The present disclosure provides for methods, systems, and compositions of nucleic acid and peptide sequences. For example, the present disclosure provides for one or more polynucleotide sequences encoding one or more amino acid sequences selected from the group consisting of SEQ ID NO: 1, SEQ ID NO: 2, SEQ ID NO: 21, SEQ ID NO: 23, SEQ ID NO: 86, SEQ ID NO: 88, SEQ ID NO: 125, SEQ ID NO: 154, SEQ ID NO: 194, SEQ ID NO: 200, SEQ ID NO: 209, and SEQ ID NO: 366.

This application is a continuation of U.S. patent application Ser. No. 17/815,086 filed on Jul. 26, 2022, which is a divisional of U.S. patent application Ser. No. 17/551,679 filed on Dec. 15, 2021, now U.S. Pat. No. 11,464,837, which is a continuation of U.S. patent application Ser. No. 17/336,960 filed Jun. 2, 2021, now U.S. Pat. No. 11,235,039, which is a continuation of Ser. No. 17/100,630 filed Nov. 20, 2020, now U.S. Pat. No. 11,058,751, each of which is incorporated by reference herein in its entirety. This patent disclosure contains material that is subject to copyright protection. The copyright owner has no objection to the facsimile reproduction of the patent document or the patent disclosure as it appears in the U.S. Patent and Trademark Office patent file or records, but otherwise reserves any and all copyright rights.

### INCORPORATION BY REFERENCE

All documents cited herein are incorporated herein by reference in their entirety.

### SEQUENCE LISTING

ST.26 Sequence Listing XML submitted in compliance with 37 CFR 1.831-1.834, through the USPTO's Patent Center electronic filing system. The XML Sequence Listing file was created on Jun. 11, 2025. The XML format file is 1,379,909 bytes in size and is titled “2215269_00123US5_SL.xml.”

### REFERENCE TO AN ELECTRONIC SEQUENCE LISTING

The contents of the electronic Sequence Listing file (2215269_00123US5_SL.xml) is herein incorporated by reference in its entirety.

### TECHNICAL FIELD

The present invention relates generally to compositions, systems, and methods of peptide vaccines. More particularly, the present invention relates to compositions, systems, and methods of designing peptide vaccines to treat or prevent disease optimized based on predicted population immunogenicity.

### BACKGROUND

The goal of a peptide vaccine is to train the immune system to recognize and expand its capacity to engage cells that display foreign peptides to improve the immune response to cancerous cells or pathogens. A peptide vaccine can also be administered to someone who is already diseased to increase their immune response to a causal cancer, other diseases, or pathogen. There exists a need for compositions, systems, and methods of peptide vaccines based on prediction of the foreign peptides that will be displayed at a later time to protect a host from cancer, other disease, or pathogen infection.

### SUMMARY OF THE INVENTION

In one aspect, the invention provides for a composition comprising one or more polynucleotides, wherein the one or more polynucleotides comprise one or more polynucleotide sequences encoding one or more amino acid sequences selected from the group consisting of SEQ ID NO: 1, SEQ ID NO: 2, SEQ ID NO: 21, SEQ ID NO: 23, SEQ ID NO: 86, SEQ ID NO: 88, SEQ ID NO: 125, SEQ ID NO: 154, SEQ ID NO: 194, SEQ ID NO: 200, SEQ ID NO: 209, and SEQ ID NO: 366, wherein each amino acid sequence of the one or more acid sequences encode a fragment of a KRAS protein, wherein the fragment of the KRAS protein comprises a G12D mutation and a heteroclitic modification.

In some embodiments, the one or more polynucleotides are contained in a construct for in vivo expression in a subject of one or more peptides encoded by the one or more amino acid sequences. In some embodiments, an administration of the one or more polynucleotides causes the one or more peptides encoded by the one or more amino acid sequences to be displayed by a HLA class I molecule in the subject. In some embodiments, the one or more peptides are displayed by the HLA class I molecule with a peptide-HLA binding affinity value of less than about 500 nM. In some embodiments, the one or more polynucleotide sequences encode at least two amino acid sequences selected from the group consisting of SEQ ID NO: 1, SEQ ID NO: 2, SEQ ID NO: 21, SEQ ID NO: 23, SEQ ID NO: 86, SEQ ID NO: 88, SEQ ID NO: 125, SEQ ID NO: 154, SEQ ID NO: 194, SEQ ID NO: 200, SEQ ID NO: 209, and SEQ ID NO: 366. In some embodiments, the one or more polynucleotides are contained in a construct for in vivo expression in a subject of at least two peptides encoded by the at least two amino acid sequences. In some embodiments, an administration of the one or more polynucleotides causes the at least two peptides encoded by the at least two amino acid sequences to be displayed by a HLA class I molecule in the subject. In some embodiments, the administration of the one or more polynucleotides causes: a first peptide of the at least two peptides to be displayed by a first plurality of HLA class I alleles; and a second peptide of the at least two peptides to be displayed by a second plurality of HLA class I alleles, wherein the first plurality of HLA class I alleles and the second plurality of HLA class I alleles differ by at least one HLA class I allele. In some embodiments, the composition is an immunogenic composition and is configured to prevent or treat cancer in the subject. In some embodiments, the one or more polynucleotides encode each of SEQ ID NO: 1, SEQ ID NO: 2, SEQ ID NO: 21, SEQ ID NO: 23, SEQ ID NO: 86, SEQ ID NO: 88, SEQ ID NO: 125, SEQ ID NO: 154, SEQ ID NO: 194, SEQ ID NO: 200, SEQ ID NO: 209, and SEQ ID NO: 366.

In another aspect, the invention provides for a composition comprising one or more polynucleotides, wherein the one or more polynucleotides comprise one or more polynucleotide sequences encoding one or more amino acid sequences selected from the group consisting of SEQ ID NO: 3, SEQ ID NO: 4, SEQ ID NO: 6, SEQ ID NO: 15, SEQ ID NO: 16, SEQ ID NO: 69, SEQ ID NO: 72, SEQ ID NO: 118, SEQ ID NO: 122, SEQ ID NO: 131, SEQ ID NO: 138, SEQ ID NO: 152, SEQ ID NO: 189, SEQ ID NO: 233, SEQ ID NO: 353, SEQ ID NO: 514, and SEQ ID NO: 591, wherein each amino acid sequence of the one or more amino acid sequences encodes a fragment of a KRAS protein, wherein the fragment of the KRAS protein comprises a G12V mutation and a heteroclitic modification.

In some embodiments, the one or more polynucleotides are contained in a construct for in vivo expression in a subject of one or more peptides encoded by the one or more amino acid sequences. In some embodiments, an administration of the one or more polynucleotides causes the one or more peptides encoded by the one or more amino acid sequences to be displayed by a HLA class I molecule in the subject. In some embodiments, the one or more peptides are displayed by the HLA class I molecule with a peptide-HLA binding affinity value of less than about 500 nM. In some embodiments, the one or more polynucleotide sequences encode at least two amino acid sequence selected from the group consisting of SEQ ID NO: 3, SEQ ID NO: 4, SEQ ID NO: 6, SEQ ID NO: 15, SEQ ID NO: 16, SEQ ID NO: 69, SEQ ID NO: 72, SEQ ID NO: 118, SEQ ID NO: 122, SEQ ID NO: 131, SEQ ID NO: 138, SEQ ID NO: 152, SEQ ID NO: 189, SEQ ID NO: 233, SEQ ID NO: 353, SEQ ID NO: 514, and SEQ ID NO: 591. In some embodiments, the one or more polynucleotides are contained in a construct for in vivo expression in a subject of at least two peptides encoded by the at least two amino acid sequence. In some embodiments, an administration of the one or more polynucleotides causes the at least two peptides encoded by the at least two amino acid sequences to be displayed by a HLA class I molecule in the subject. In some embodiments, the administration of the one or more polynucleotides causes: a first peptide of the at least two peptides to be displayed by a first plurality of HLA class I alleles; and a second peptide of the at least two peptides to be displayed by a second plurality of HLA class I alleles, wherein the first plurality of HLA class I alleles and the second plurality of HLA class I alleles differ by at least one HLA class I allele. In some embodiments, the composition is an immunogenic composition and is configured to prevent or treat cancer in the subject. In some embodiments, the one or more polynucleotides encode each of SEQ ID NO: 3, SEQ ID NO: 4, SEQ ID NO: 6, SEQ ID NO: 15, SEQ ID NO: 16, SEQ ID NO: 69, SEQ ID NO: 72, SEQ ID NO: 118, SEQ ID NO: 122, SEQ ID NO: 131, SEQ ID NO: 138, SEQ ID NO: 152, SEQ ID NO: 189, SEQ ID NO: 233, SEQ ID NO: 353, SEQ ID NO: 514, and SEQ ID NO: 591.

In another aspect, the invention provides for a composition comprising one or more polynucleotides, wherein the one or more polynucleotides comprise one or more polynucleotide sequences encoding one or more amino acid sequences selected from the group consisting of SEQ ID NO: 5, SEQ ID NO: 8, SEQ ID NO: 9, SEQ ID NO: 10, SEQ ID NO: 17, SEQ ID NO: 18, SEQ ID NO: 19, SEQ ID NO: 20, SEQ ID NO: 25, SEQ ID NO: 113, SEQ ID NO: 181, SEQ ID NO: 195, SEQ ID NO: 212, and SEQ ID NO: 324, wherein each amino acid sequence of the one or more amino acid sequences encodes a fragment of a KRAS protein, wherein the fragment of the KRAS protein comprises a G12R mutation and a heteroclitic modification.

In some embodiments, the one or more polynucleotides are contained in a construct for in vivo expression in a subject of one or more peptides encoded by the one or more amino acid sequences. In some embodiments, an administration of the one or more polynucleotides causes the one or more peptides encoded by the one or more amino acid sequences to be displayed by a HLA class I molecule in the subject. In some embodiments, the one or more peptides are displayed by the HLA class I molecule with a peptide-HLA binding affinity value of less than about 500 nM. In some embodiments, the one or more polynucleotide sequences encode at least two amino acid sequence selected from the group consisting of SEQ ID NO: 5, SEQ ID NO: 8, SEQ ID NO: 9, SEQ ID NO: 10, SEQ ID NO: 17, SEQ ID NO: 18, SEQ ID NO: 19, SEQ ID NO: 20, SEQ ID NO: 25, SEQ ID NO: 113, SEQ ID NO: 181, SEQ ID NO: 195, SEQ ID NO: 212, and SEQ ID NO: 324. In some embodiments, the one or more polynucleotides are contained in a construct for in vivo expression in a subject of at least two peptides encoded by the at least two amino acid sequence. In some embodiments, an administration of the one or more polynucleotides causes the at least two peptides encoded by the at least two amino acid sequences to be displayed by a HLA class I molecule in the subject. In some embodiments, the administration of the one or more polynucleotides causes: a first peptide of the at least two peptides to be displayed by a first plurality of HLA class I alleles; and a second peptide of the at least two peptides to be displayed by a second plurality of HLA class I alleles, wherein the first plurality of HLA class I alleles and the second plurality of HLA class I alleles differ by at least one HLA class I allele. In some embodiments, the composition is an immunogenic composition and is configured to prevent or treat cancer in the subject. In some embodiments, the one or more polynucleotides encode each of SEQ ID NO: 5, SEQ ID NO: 8, SEQ ID NO: 9, SEQ ID NO: 10, SEQ ID NO: 17, SEQ ID NO: 18, SEQ ID NO: 19, SEQ ID NO: 20, SEQ ID NO: 25, SEQ ID NO: 113, SEQ ID NO: 181, SEQ ID NO: 195, SEQ ID NO: 212, and SEQ ID NO: 324.

In another aspect, the invention provides for a composition comprising one or more polynucleotides, wherein the one or more polynucleotides comprise one or more polynucleotide sequences encoding one or more amino acid sequences selected from the group consisting of SEQ ID NO: 26, SEQ ID NO: 27, SEQ ID NO: 28, SEQ ID NO: 30, SEQ ID NO: 36, SEQ ID NO: 37, SEQ ID NO: 70, SEQ ID NO: 71, SEQ ID NO: 73, SEQ ID NO: 74, SEQ ID NO: 78, SEQ ID NO: 176, and SEQ ID NO: 187, wherein each amino acid sequence of the one or more amino acid sequences encodes a fragment of a KRAS protein, wherein the fragment of the KRAS protein comprises a G12C mutation and a heteroclitic modification.

In some embodiments, the one or more polynucleotides are contained in a construct for in vivo expression in a subject of one or more one peptides encoded by the one or more amino acid sequences. In some embodiments, an administration of the one or more polynucleotides causes the one or more peptides encoded by the one or more amino acid sequences to be displayed by a HLA class I molecule in the subject. In some embodiments, the one or more peptides are displayed by the HLA class I molecule with a peptide-HLA binding affinity value of less than about 500 nM. In some embodiments, the one or more polynucleotide sequences encode at least two amino acid sequence selected from the group consisting of SEQ ID NO: 26, SEQ ID NO: 27, SEQ ID NO: 28, SEQ ID NO: 30, SEQ ID NO: 36, SEQ ID NO: 37, SEQ ID NO: 70, SEQ ID NO: 71, SEQ ID NO: 73, SEQ ID NO: 74, SEQ ID NO: 78, SEQ ID NO: 176, and SEQ ID NO: 187. In some embodiments, the one or more polynucleotides are contained in a construct for in vivo expression in a subject of at least two peptides encoded by the at least two amino acid sequence. In some embodiments, an administration of the one or more polynucleotides causes the at least two peptides encoded by the at least two amino acid sequences to be displayed by a HLA class I molecule in the subject. In some embodiments, the administration of the one or more polynucleotides causes: a first peptide of the at least two peptides to be displayed by a first plurality of HLA class I alleles; and a second peptide of the at least two peptides to be displayed by a second plurality of HLA class I alleles, wherein the first plurality of HLA class I alleles and the second plurality of HLA class I alleles differ by at least one HLA class I allele. In some embodiments, the composition is an immunogenic composition and is configured to prevent or treat cancer in the subject. In some embodiments, the one or more polynucleotides encode each of SEQ ID NO: 26, SEQ ID NO: 27, SEQ ID NO: 28, SEQ ID NO: 30, SEQ ID NO: 36, SEQ ID NO: 37, SEQ ID NO: 70, SEQ ID NO: 71, SEQ ID NO: 73, SEQ ID NO: 74, SEQ ID NO: 78, SEQ ID NO: 176, and SEQ ID NO: 187.

In one aspect, the invention provides for a nucleic acid sequence encoding two or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In some embodiments, the nucleic acid sequence is an immunogenic composition. In some embodiments, the nucleic acid sequence is administered in a construct for expression in vivo. In some embodiments, the in vivo administration of the nucleic acid sequence is configured to produce one or more peptides that are displayed by an HLA class I molecule. In some embodiments, the one or more peptides is a modified or unmodified fragment of a mutated KRAS protein. In some embodiments, the mutated KRAS protein is selected from the group consisting of KRAS G12D, KRAS G12V, and KRAS G12R. In some embodiments, the nucleic acid sequence is administered in an effective amount to a subject to prevent cancer. In some embodiments, the nucleic acid sequence is administered in an effective amount to a subject to treat cancer.

In another aspect, the invention provides for an immunogenic peptide composition comprising two or more peptides selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In some embodiments, a peptide in the immunogenic peptide composition is displayed by an HLA class I molecule. In some embodiments, a peptide in the immunogenic peptide composition is a modified or unmodified fragment of a mutated KRAS protein. In some embodiments, the mutated KRAS protein is selected from the group consisting of KRAS G12D, KRAS G12V, and KRAS G12R. In some embodiments, the immunogenic peptide composition is administered in an effective amount to a subject to prevent cancer. In some embodiments, the immunogenic peptide composition is administered in an effective amount to a subject to treat cancer. In some embodiments, the immunogenic peptide composition comprises at least three peptides selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In another aspect, the invention provides for a nucleic acid sequence encoding one or more amino acid sequences selected from the group consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

In some embodiments, the nucleic acid sequence is an immunogenic composition. In some embodiments, the nucleic acid sequence is administered in a construct for expression in vivo. In some embodiments, the in vivo administration of the nucleic acid sequence is configured to produce one or more peptides that are displayed by an HLA class II molecule. In some embodiments, the one or more peptides is a modified fragment of a mutated KRAS protein. In some embodiments, the mutated KRAS protein is selected from the group consisting of KRAS G12D, KRAS G12V, KRAS G12R, KRAS G12C, and KRAS G13D. In some embodiments, the nucleic acid sequence is administered in an effective amount to a subject to prevent cancer. In some embodiments, the nucleic acid sequence is administered in an effective amount to a subject to treat cancer.

In another aspect, the invention provides for an immunogenic peptide composition comprising one or more peptides selected from the group consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

In some embodiments, a peptide in the immunogenic peptide composition is displayed by an HLA class II molecule. In some embodiments, a peptide in the immunogenic peptide composition is a modified or unmodified fragment of a mutated KRAS protein. In some embodiments, the mutated KRAS protein is selected from the group consisting of KRAS G12D, KRAS G12V, KRAS G12R, KRAS G12C, and KRAS G13D. In some embodiments, the immunogenic peptide composition is administered in an effective amount to a subject to prevent cancer. In some embodiments, the immunogenic peptide composition is administered in an effective amount to a subject to treat cancer. In some embodiments, the immunogenic peptide composition comprises at least two peptides selected from the group consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

### BRIEF DESCRIPTION OF THE DRAWINGS

The following figures depict illustrative embodiments of the invention.

FIG. 1 is a flow chart of a vaccine optimization method.

FIG. 2 is a flow chart of vaccine optimization method with seed set compression.

FIG. 3 shows predicted population coverage for single target MHC class I vaccines by vaccine size for KRAS G12D, KRAS G12V, KRAS G12R, KRAS G12C, and KRAS G13D targets.

FIG. 4 shows predicted population coverage for single target MHC class II vaccines by vaccine size for KRAS G12D, KRAS G12V, KRAS G12R, KRAS G12C, and KRAS G13D targets.

FIG. 5 shows probabilities of disease presentations for pancreas, colon/rectum, and bronchus/lung and respective probabilities of target presentations for KRAS G12D, KRAS G12V, and KRAS G12R targets.

FIG. 6 is a flow chart for multiple target (combined) vaccine optimization methods.

FIG. 7 shows predicted population coverage for pancreatic cancer multiple target (combined) MHC class I vaccines by vaccine size for KRAS G12D, KRAS G12V, and KRAS G12R targets.

FIG. 8 shows predicted population coverage for pancreatic cancer multiple target (combined) MHC class II vaccines by vaccine size for KRAS G12D, KRAS G12V, and KRAS G12R targets.

FIG. 9 shows an example Python implementation of the MergeMulti function for combined vaccine design procedures.

FIG. 10 shows predicated peptide-HLA hits by vaccine size for a KRAS G12V vaccine for the HLA diplotype HLA-A02:03, HLA-A11:01, HLA-B55:02, HLA-B58:01, HLA-C03:02, HLA-C03:03.

### DETAILED DESCRIPTION

In some embodiments, the disclosure provides for peptide vaccines that incorporate peptide sequences that will be displayed by Major Histocompatibility Complex (MHC) molecules on cells and train the immune system to recognize cancer or pathogen diseased cells. In some embodiments, a peptide vaccine is a composition that consists of one or more peptides. In some embodiments, a peptide vaccine is an mRNA or DNA construct administered for expression in vivo that encodes for one or more peptides.

Peptide display by an MHC molecule is necessary, but not sufficient, for a peptide to be immunogenic and cause the recognition of the resulting peptide-MHC complex by an individual's T cells to trigger T cell activation, expansion, and immune memory. In some embodiments, experimental data from assays such as the ELISPOT (Slota et al., 2011) or the Multiplex Identification of Antigen-Specific T Cell Receptors Using a Combination of Immune Assays and Immune Receptor Sequencing (MIRA) assay (Klinger et al., 2015) can be combined with machine learning based predictions of peptide display by an MHC molecule. In some embodiments, the MHCflurry or NetMHCpan computational methods (known in the art) are used to predict MHC class I display of a peptide by an HLA allele (see Table 1). In some embodiments, the NetMHCIIpan computational method (known in the art) is used to predict MHC class II display of a peptide by an HLA allele (see Table 2).

A peptide is displayed by an MHC molecule when it binds within the groove of the MHC molecule and is transported to the cell surface where it can be recognized by a T cell receptor. In some embodiments, a peptide that is part of the normal proteome in a healthy individual is a self-peptide, and a peptide that is not part of the normal proteome is a foreign peptide. Foreign peptides can be generated by mutations in normal self-proteins in tumor cells that create epitopes called neoantigens, or by pathogenic infections. In some embodiments, a neoantigen is any subsequence of a human protein, where the subsequence contains one or more altered amino acids or protein modifications that do not appear in a healthy individual.

For example, KRAS gene mutations are the most frequently mutated oncogenes in cancer, but they have been very difficult to treat with small molecule therapeutics. The KRAS protein is part of a signaling pathway that controls cellular growth, and point mutations in the protein can cause constitutive pathway activation and uncontrolled cell growth. Single amino acid KRAS mutations result in minor changes in protein structure, making it difficult to engineer small molecule drugs that recognize a mutant specific binding pocket and inactivate KRAS signaling. KRAS oncogenic mutations include the mutation of position 12 from glycine to aspartic acid (G12D), glycine to valine (G12V), glycine to arginine (G12R), or glycine to cystine (G12C); or the mutation of position 13 from glycine to aspartic acid (G13D). The corresponding foreign peptides contain these mutations.

A challenge for the design of peptide vaccines is the diversity of human MHC alleles that each have specific preferences for the peptide sequences they will display. The Human Leukocyte Antigen (HLA) loci, located within the MHC, encode the HLA class I and class II molecules. There are three classical class I loci (HLA-A, HLA-B, and HLA-C) and three loci that encode class II molecules (HLA-DR, HLA-DQ, and HLA-DP). An individual's HLA type describes the alleles they carry at each of these loci. Peptides of length of between about 8 and about 11 residues can bind to HLA class I (or MHC class I) molecules whereas those of length of between about 13 and about 25 bind to HLA class II (or MHC class II) molecules (Rist et al., 2013; Chicz et al., 1992). Human populations that originate from different geographies have differing frequencies of HLA alleles, and these populations exhibit linkage disequilibrium between HLA loci that result in population specific haplotype frequencies. In some embodiments, methods are disclosed for creating effective vaccines that includes consideration of the HLA allelic frequency in the target population, as well as linkage disequilibrium between HLA genes to achieve a set of peptides that is likely to be robustly displayed.

The present disclosure provides for compositions, systems, and methods of vaccine designs that produce immunity to multiple targets. In some embodiments, a target is a neoantigen protein sequence, a pathogen proteome, or any other undesired protein sequence that is non-self and is expected to be bound and displayed by an MHC molecule. When a target is present in an individual it may result in multiple peptide sequences that are displayed by a variety of HLA alleles. Therefore, in this disclosure, “foreign peptide” refers to an amino acid sequence encoding a fragment of a target protein/peptide (or a full protein/peptide), the target protein/peptide consisting of: a neoantigen protein, a pathogen proteome, or any other undesired protein that is non-self and is expected to be bound and displayed by an MHC molecule.

In some embodiments, peptide-MHC immunogenicity data or computational predictions of peptide-MHC immunogenicity can be included and combined with scores for peptide display in the methods disclosed herein. One way of combining the scores is using immunogenicity data for peptides assayed for immunogenicity in diseased or vaccinated individuals, and assigning peptides to the HLA allele that displayed them in the individual by choosing the HLA allele that computational methods predict has the highest likelihood of display. For peptides that are not experimentally assayed, computational predictions of display can be used.

Since immunogenicity may vary from individual to individual, one method to increase the probability of vaccine efficacy is to use a diverse set of foreign peptides (e.g., at least two peptides) to increase the chances that some subset of them will be immunogenic in a given individual. Prior research using mouse models has shown that most MHC displayed peptides are immunogenic, but immunogenicity varies from individual to individual as described in Croft et al. (2019). In some embodiments, experimental peptide-HLA immunogenicity data are used to determine which foreign peptides and their modifications will be effective immunogens in a vaccine.

Considerations for the design of peptide vaccines are outlined in Liu et al., Cell Systems 11, Issue 2, p. 131-146 (Liu et al., 2020) and (Liu et al., 2020b) which are incorporated by reference herein.

Certain foreign peptides may not bind with high affinity to a wide range of HLA molecules. To increase the binding of foreign peptides to HLA molecules, their amino acid composition can be altered to change one or more anchor residues or other residues. Anchor residues are amino acids that interact with an HLA molecule and have the largest influence on the affinity of a peptide for an HLA molecule. Peptides with altered anchor residues are called heteroclitic peptides. In some embodiments, heteroclitic peptides include foreign peptides with residue modifications at non-anchor positions. In some embodiments, heteroclitic peptides include foreign peptides with residue modifications that include unnatural amino acids and amino acid derivatives. Modifications to create heteroclitic peptides can improve the binding of peptides to both MHC class I and MHC class II molecules, and the modifications required can be both peptide and MHC class specific. Since peptide anchor residues face the MHC molecule groove, they are less visible than other peptide residues to T cell receptors. Thus, heteroclitic peptides have been observed to induce a T cell response where the stimulated T cells also respond to unmodified peptides. It has been observed that the use of heteroclitic peptides in a vaccine can improve a vaccine's effectiveness (Zirlik et al., 2006). In some embodiments, the immunogenicity of heteroclitic peptides are experimentally determined and their ability to activate T cells that also recognize the corresponding base (also called seed) peptide of the heteroclitic peptide is performed as is known in the art. In some embodiments, these assays of the immunogenicity and cross-reactivity of heteroclitic peptides are performed when the heteroclitic peptides are displayed by specific HLA alleles.

### Peptide Vaccines to Induce Immunity to One or More Targets

In some embodiments, a method is provided for formulating peptide vaccines using a single vaccine design for a one or more targets. In some embodiments, a single target is a foreign protein with a specific mutation (e.g., KRAS G12D). In some embodiments, multiple targets can be used (e.g. both KRAS G12D and KRAS G13D).

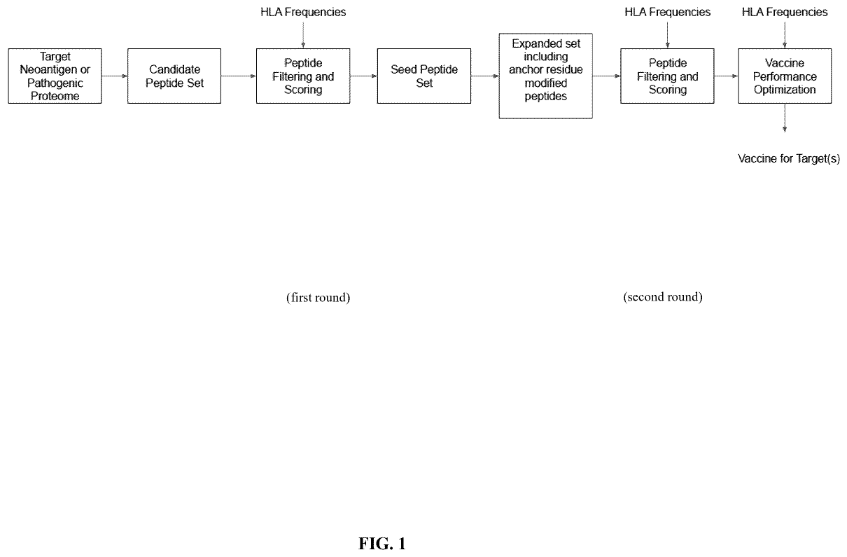

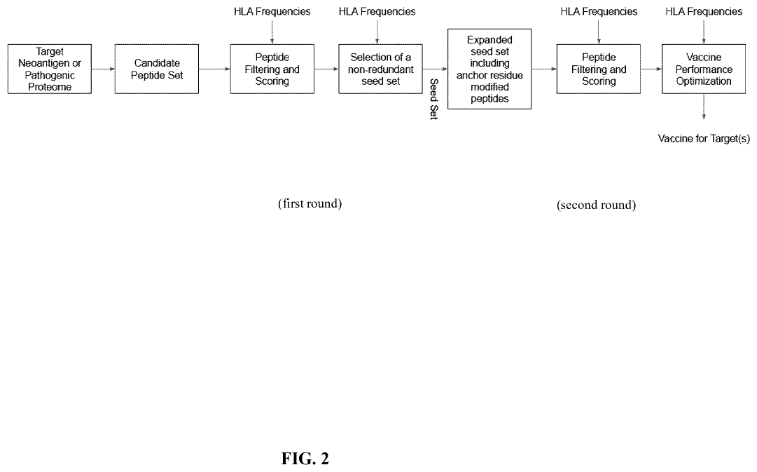

In some embodiments, the method includes extracting peptides to construct a candidate set from all target proteome sequences (e.g., entire KRAS G12D protein) as described in Liu et al. (2020). FIGS. 1 and 2 depict flow charts for example vaccine design methods that can be used for MHC class I or MHC class II vaccine design. In some embodiments, extracted foreign peptides are of amino acid length of between about 8 and about 10 (e.g., for MHC class I binding (Rist et al., 2013)). In some embodiments, the extracted foreign peptides presented by MHC class I molecules are longer than 10 amino acid residues, such as 11 residues (Trolle et al., 2016). In some embodiments, extracted foreign peptides are of length between about 13 and about 25 (e.g., for class II binding (Chicz et al., 1992)). In some embodiments, sliding windows of various size ranges described herein are used over the entire proteome. In some embodiments, other foreign peptide lengths for MHC class I and class II sliding windows can be utilized. In some embodiments, computational predictions of proteasomal cleavage are used to filter or select peptides in the candidate set. One computational method for predicting proteasomal cleavage is described by Nielsen et al. (2005). In some embodiments, peptide mutation rates, glycosylation, cleavage sites, or other criteria can be used to filter peptides as described in Liu et al. (2020).

As shown in FIGS. 1-2, in some embodiments, the next step of the method includes scoring the foreign peptides in the candidate set for binding to all considered HLA alleles as described in Liu et al. (2020) and Liu et al. (2020b). Scoring can be accomplished for human HLA molecules, mouse H-2 molecules, swine SLA molecules, or MHC molecules of any species for which prediction algorithms are available or can be developed. Thus, vaccines targeted at non-human species can be designed with the method. Scoring metrics can include the affinity for a foreign peptide to an HLA allele in nanomolar, eluted ligand, presentation, and other scores that can be expressed as percentile rank or any other metric. The candidate set may be further filtered to exclude peptides whose predicted binding cores do not contain a particular pathogenic or neoantigen target residue of interest or whose predicted binding cores contain the target residue in an anchor position. The candidate set may also be filtered for foreign peptides of specific lengths, such as length 9 for MHC class I, for example. In some embodiments, scoring of foreign peptides is accomplished with experimental data or a combination of experimental data and computational prediction methods.

The criteria used for scoring peptide-HLA binding during the scoring procedure can accommodate different goals during the candidate identification and vaccine design phases. For example, a foreign peptide with peptide-HLA binding affinities of 500 nM may be displayed by an individual that is diseased, but at a lower frequency than a foreign peptide with a 50 nM peptide-HLA binding affinity. Thus, during the scoring of a candidate set to qualify potential immune system targets, 500 nM or other less constrained affinity criteria than 50 nM may be utilized. During the combinatorial design phase of a vaccine, a more constrained affinity criteria may be used, such a 50 nM, to increase the probability that a vaccine peptide will be found and displayed by HLA molecules. In some embodiments, peptides are selected that have peptide-HLA binding affinities of between about 50 nM and about 500 nM. Alternatively, combined models that incorporate peptide immunogenicity can be used to qualify foreign peptides for improvement and score their modified versions for vaccine inclusion. In some embodiments, experimental observations of the immunogenicity of peptides in the context of their display by HLA alleles can be used to score peptides for vaccine inclusion. In some embodiments, computational predictions of the immunogenicity of a peptide in the context of display by HLA alleles can used for scoring such as the methods of Ogishi et al. (2019).

In some embodiments, the method further includes running the OptiVax-Robust algorithm as described in Liu et al. (2020) using the HLA haplotype frequencies of a population on the scored candidate set to construct a seed set (also referred to as base set herein) of foreign peptides (FIG. 2). In some embodiments, HLA diplotype frequencies can be provided to OptiVax. OptiVax-Robust includes algorithms to eliminate peptide redundancy that arises from the sliding window approach with varying window sizes, but other redundancy elimination measures can be used to enforce minimum edit distance constraints between foreign peptides in the candidate set. The size of the seed set is determined by a point of diminishing returns of population coverage as a function of the number of foreign peptides in the seed set. Other criteria can also be used, including a minimum number of vaccine foreign peptides, maximum number of vaccine foreign peptides, and desired predicted population coverage. One alternative criterion is a minimum number of expected peptide-HLA hits in each individual, where a peptide-HLA hit is the potential immunogenic display of a peptide by a single HLA allele as described as in Liu et al. (2020b). In alternate embodiments, the method further includes running the OptiVax-Unlinked algorithm as described in Liu et al. (2020) instead of OptiVax-Robust.

The OptiVax-Robust method uses binary predictions of foreign peptide binding to HLA alleles, and these binary predictions can be generated as described in Liu et al. (2020). The OptiVax-Unlinked method uses the probability of foreign peptide binding to HLA alleles and can be generated as described in Liu et al. (2020). Either method can be used for the purposes described herein, and thus we will the term “OptiVax” refers to either the Robust or Unlinked method. In some embodiments, the observed probability of peptide-HLA immunogenicity in experimental assays can be used as the probability of peptide-HLA binding in EvalVax-Unlinked and OptiVax-Unlinked. In some embodiments, the HLA haplotype or HLA allele frequencies of a population provided to OptiVax for vaccine design describe the world's population. In alternative embodiments, the HLA haplotype or HLA allele frequencies of a population provided to OptiVax for vaccine design are specific to a geographic region. In alternative embodiments, the HLA haplotype or HLA allele frequencies of a population provided to OptiVax for vaccine design are specific to an ancestry. In alternative embodiments, the HLA haplotype or HLA allele frequencies of a population provided to OptiVax for vaccine design are specific to a race. In alternative embodiments, the HLA haplotype or HLA allele frequencies of a population provided to OptiVax for vaccine design are specific to individuals with risk factors such as genetic indicators of risk, age, exposure to chemicals, alcohol use, chronic inflammation, diet, hormones, immunosuppression, infectious agents, obesity, radiation, sunlight, or tobacco use. In alternative embodiments, the HLA haplotype or HLA allele frequencies of a population provided to OptiVax for vaccine design are specific to individuals that carry certain HLA alleles. In alternative embodiments, the HLA diplotypes provided to OptiVax for vaccine design describe a single individual, and are used to design an individualized vaccine.

In some embodiments, the seed set of foreign peptides that results from OptiVax application to the candidate set of target peptides describes a set of unmodified foreign peptides that represent a possible compact vaccine design (Seed Set in FIG. 2). In some embodiments, the seed set is based upon filtering candidate peptides by predicted or observed affinity or immunogenicity with respect to HLA molecules (Seed Set in FIG. 1). However, to improve the display of the foreign peptides in a wide range of HLA haplotypes as possible, some embodiments include modifications of the seed (or base) set. In some embodiments, experimental assays can be used to ensure that a modified seed (or base) peptide activates T cells that also recognize the seed peptide.

For a given foreign peptide, the optimal anchor residue selection may depend upon the HLA allele that is binding to and displaying the foreign peptide and the class of the HLA allele (MHC class I or class II). A seed peptide set can become an expanded set by including anchor residue modified peptides of either MHC class I or II peptides (FIGS. 1-2). Thus, one aspect of vaccine design is considering how to select a limited set of heteroclitic peptides that derive from the same foreign peptide for vaccine inclusion given that different heteroclitic peptides will have different and potentially overlapping population coverages.

In some embodiments, all possible anchor modifications for each base set foreign peptide are considered. There are two anchor residues in peptides bound by MHC class I molecules, typically at positions 2 and 9 for 9-mer peptides. At each anchor position, 20 possible amino acids are attempted in order to select the best heteroclitic peptides. Thus, for MHC class I binding, 400 (i.e., 20 amino acids by 2 positions=20²) minus 1 heteroclitic peptides are generated for each base foreign peptide. There are four anchor residues in peptides bound by MHC class II molecules, typically at positions 1, 4, 6, and 9 of the 9-mer binding core. Thus, for MHC class II binding there are 160,000 (i.e., 20 amino acids by 4 positions=204) minus 1 heteroclitic peptides generated for each base foreign peptide. Other methods, including Bayesian optimization, can be used to select optimal anchor residues to create heteroclitic peptides from each seed (or base) set peptide. Other methods are presented in “Machine learning optimization of peptides for presentation by class II MHCs” by Dai et al. (2020), incorporated in its entirety herein. In some embodiments, the anchor positions are determined by the HLA allele that presents a peptide, and thus the set of heteroclitic peptides includes for each set of HLA specific anchor positions, all possible anchor modifications.

In some embodiments, for all of the foreign peptides in the seed set, new peptide sequences with all possible anchor residue modifications (e.g., MHC class I or class II) are created resulting in a new heteroclitic base set (Expanded set in FIGS. 1-2) that includes all of the modifications. In some embodiments, the heteroclitic base set (Expanded set in FIGS. 1-2) also includes the original seed (or base) set (Seed Peptide Set in FIGS. 1-2). In some embodiments, the heteroclitic base set includes amino acid substitutions or non-natural amino acid analogs at non-anchor residues. The heteroclitic base set is scored for HLA affinity or other metrics as described herein (another round of Peptide Filtering and Scoring as shown in FIGS. 1-2). The scoring predictions may be further updated for pairs of heteroclitic peptide and HLA allele, eliminating pairs where a heteroclitic peptide is predicted to be displayed by an allele but the seed (or base) peptide from which it was derived is not predicted to be displayed by the allele. The scoring predictions may also be filtered to ensure that predicted binding cores of the heteroclitic peptide displayed by a particular HLA allele align exactly in position with the binding cores of the respective seed (or base) set foreign peptide for that HLA allele. In some embodiments the scoring predictions are filtered for an HLA allele to ensure that the heteroclitic peptides considered for that HLA allele are only modified at anchor positions determined by that HLA allele. In some embodiments, heteroclitic peptides are included in experimental assays such as MIRA (Klinger et al., 2015) to determine their immunogenicity with respect to specific HLA alleles. In some embodiments, the methods of Liu et al. (2020b), can be used to incorporate MIRA data for heteroclitic peptides into a model of peptide-HLA immunogenicity. In some embodiments, the immunogenicity of heteroclitic peptides are experimentally determined and their ability to activate T cells that also recognize the corresponding seed (or base) peptide of the heteroclitic peptide is performed as is known in the art. In some embodiments, these assays of the immunogenicity and cross-reactivity of heteroclitic peptides are performed when the heteroclitic peptides are displayed by specific HLA alleles. In some embodiments, computational predictions of the immunogenicity of a heteroclitic peptide in the context of display by HLA alleles can used for scoring such as the methods of Ogishi et al. (2019).

In some embodiments, the next step involves inputting heteroclitic base set (also referred to as Expanded set as shown in FIGS. 1-2) to OptiVax to select a compact set of vaccine peptides that maximizes vaccine performance (Vaccine Performance Optimization; FIGS. 1-2). Vaccine performance is the population coverage of a vaccine, or the expected number peptide-HLA hits produced by a vaccine, or a function of population coverage and expected number of peptide-HLA hits desired. In some embodiments, the vaccine immunogenicity metric is a metric that describes the overall immunogenic properties of a vaccine with two or more peptides. In some embodiments, the methods described herein are included for running OptiVax. In some embodiments, population coverage means the proportion of a subject population that presents one or more immunogenic peptides that activate T cells responsive to a seed foreign peptide. The metric of population coverage is computed using the HLA haplotype frequency in a given population such as a representative human population. In some embodiments, the metric of population coverage is computed using marginal HLA frequencies in a population. Maximizing population coverage means selecting a foreign peptide set that collectively results in the greatest fraction of the population that has at least a minimum number (e.g., 1, 2, 3, 4, 5, 6, 7, or 8) of immunogenic peptide-HLA bindings based on proportions of HLA haplotypes in a given population (e.g., representative healthy or diseased human population). In some embodiments, this process includes the OptiVax selection of heteroclitic peptides (as described in this disclosure) that activate T cells that respond to their corresponding seed (or base) peptide and the heteroclitic base peptides to improve population coverage. In some embodiments, the seed (or base) foreign peptides are always included in the final vaccine design to guard against the possibility that heteroclitic peptides will not produce immunity that reacts with the native seed (or base) foreign peptides. In some embodiments, peptides are only considered as candidates for a vaccine design if they have been observed to be immunogenic in clinical data or animal models.

In some embodiments, a candidate vaccine peptide is eliminated from vaccine inclusion if it activates T cells that recognize self-peptides (e.g., this can be achieved at the first and/or second round of Peptide Filtering and Sorting as shown in FIGS. 1-2). Testing a vaccine peptide for its ability to activate T cells that recognize self-peptides can be experimentally accomplished by the vaccination of animal models followed by ELISPOT or other immunogenicity assay or with human tissue protocols. In both cases, models with HLA alleles that present the vaccine peptide are used. In some embodiments, human primary blood mononuclear cells (PBMCs) are stimulated with a vaccine peptide, the T cells are allowed to grow, and then T cell activation with a self-peptide is assayed as described in Tapia-Calle et al. (2019) or other methods as known in the art. In some embodiments, computational predictions of the ability of a peptide to activate T cells that also recognize self-peptides can be utilized. These predictions can be based upon the modeling of the outward facing residues from the peptide-HLA complex and their interactions with other peptide residues. In some embodiments, a candidate vaccine peptide is eliminated from vaccine inclusion or experimentally tested for cross-reactivity if it is predicted to activate T cells that also recognize self-peptides based upon the structural similarity of the peptide-MHC complex of the candidate peptide and the peptide-MHC complex of a self-peptide. One method for the prediction of peptide-MHC structure is described by Park et al. (2013).

In some embodiments, a candidate heteroclitic vaccine peptide is eliminated from vaccine inclusion if it does not activate T cells that recognize its corresponding seed foreign peptide (second round of Peptide Filtering and Scoring, FIGS. 1-2). Testing a candidate heteroclitic peptide for its ability to activate T cells that recognize its corresponding seed (or base) foreign peptide can be experimentally accomplished by the vaccination of animal models followed by ELISPOT or other immunogenicity assay or with human tissue protocols. In both cases, models with HLA alleles that present the heteroclitic peptide are used. In some embodiments, human PBMCs are stimulated with the heteroclitic peptide, the T cells are allowed to grow, and then T cell activation with the seed (or base) foreign peptide is assayed as described in Tapia-Calle et al. (2019) or using other methods known in the art. In some embodiments, computational predictions of the ability of a heteroclitic peptide to activate T cells that also recognize the corresponding seed (or base) foreign peptide can be utilized. These predictions can be based upon the modeling of the outward facing residues from the peptide-HLA complex and their interactions with other peptide residues. In some embodiments, the structural similarity of the peptide-MHC complex of a heteroclitic peptide and the peptide-MHC complex of the corresponding seed (or base) foreign is used to qualify heteroclitic peptides for vaccine inclusion or to require experimental immunogenicity testing before vaccine inclusion.

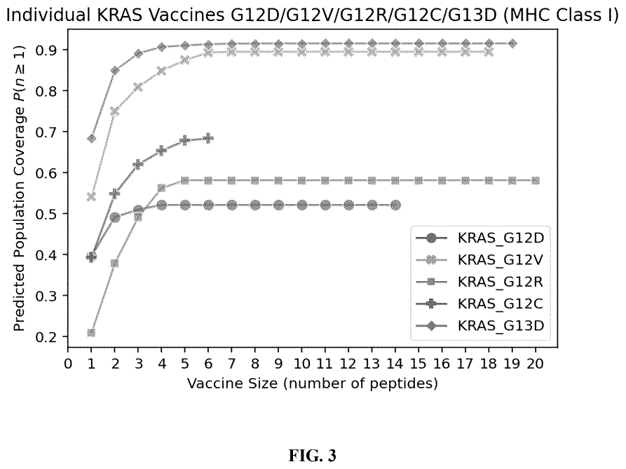

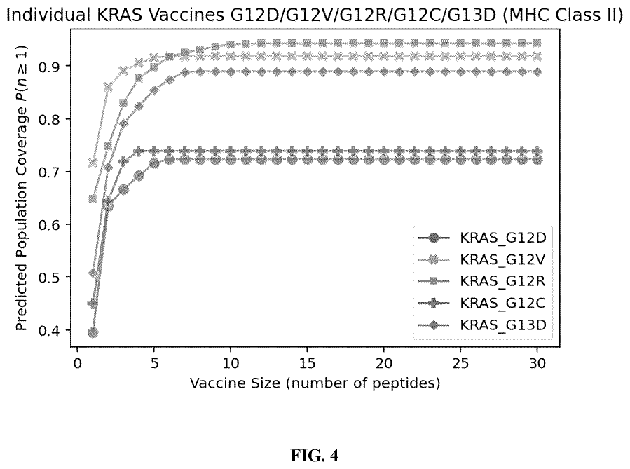

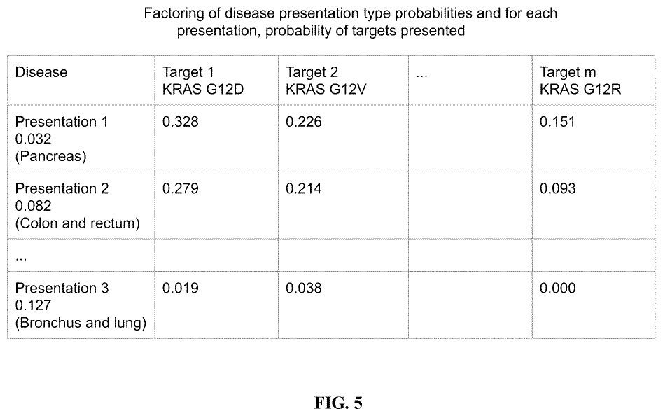

FIG. 3 (MHC class I) and FIG. 4 (MHC class II) show the predicted population coverage of OptiVax-Robust selected single target-specific vaccines with differing number of peptides designed for the KRAS mutations G12D, G12V, G12R, G12C, and G13D. FIGS. 4-5 show that as the number of peptides increases for a vaccine, its predicted population coverage increases. The population coverage shown in FIGS. 4-5 are of those individuals that have the specific mutation that the vaccine is designed to cover. An increase in peptide count will also typically cause the average number of peptide-HLA hits in each individual to increase in the population.

OptiVax can be used to design a vaccine to maximize the fraction/proportion of the population whose HLA molecules are predicted to bind to and display at least p peptides from the vaccine. In some embodiments, this prediction includes experimental immunogenicity data to directly predict at least p peptides will be immunogenic. The number p is input to OptiVax, and OptiVax can be run multiple times with varying values for p to obtain a predicted optimal foreign peptide set for different peptide counts p. Larger values of p will increase the redundancy of a vaccine at the cost of more peptides to achieve a desired population coverage. In some embodiments, it may not be possible to achieve a given population coverage given a specific heteroclitic base set. In some embodiments, the number p is a function of the desired size of a vaccine.

The methods described herein can be used to design separate vaccine formulations for MHC class I and class II based immunity.

In some embodiments, this procedure is used to create a vaccine for an individual. In some embodiments, the foreign peptides present in the individual are determined by sequencing the individual's tumor RNA or DNA, and identifying mutations that produce foreign peptides. One embodiment of this is described in U.S. Ser. No. 10/738,355B2. In some embodiments, peptide sequencing methods are used to identify foreign peptides in the individual. One embodiment of this is described in US20110257890A1. In some embodiments, the foreign peptides used for the individual's vaccine are selected when a foreign peptide or foreign peptide encoding RNA observed in a specimen from the individual is present at a predetermined level. The foreign peptides in the individual are used to construct a vaccine as described in the disclosure herein. For vaccine design OptiVax is provided a diplotype comprising the HLA type of the individual. In an alternative embodiment, the HLA type of an individual is separated into multiple diplotypes with frequencies that sum to one, where each diplotype comprises one or more HLA alleles from the individual and a notation that the other allele positions should not be evaluated. The use of multiple diplotypes will cause OptiVax's objective function to increase the chance that immunogenic peptides will be displayed by all of the constructed diplotypes.

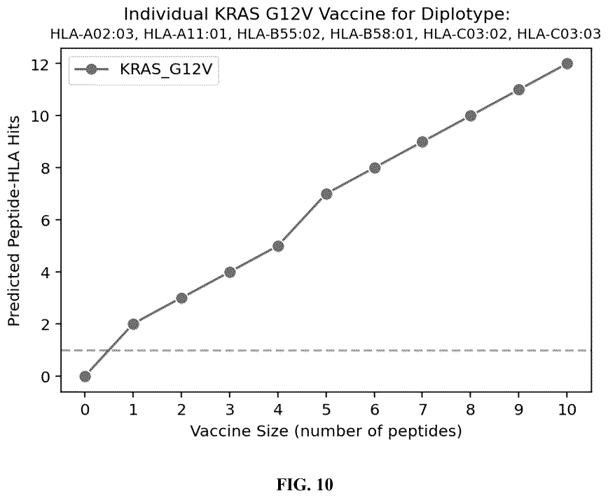

FIG. 10 shows the vaccine performance (predicted number of peptide-HLA hits) of ten example G12V MHC class I vaccines for a single individual with the MHC class I HLA diplotype HLA-A02:03, HLA-A11:01, HLA-B55:02, HLA-B58:01, HLA-C03:02, HLA-C03:03. OptiVax was used to design ten G12V MHC class I vaccines for this HLA diplotype with peptide counts ranging from 1 to 10. For the results in FIG. 10, OptiVax was run with six synthetic diplotypes, each equally weighted, each with one HLA allele from the individual's HLA diplotype, and the other allele positions marked to not be evaluated. The 10 peptide vaccine in FIG. 10 comprises SEQ ID NO: 3 (GAVGVGKSL), SEQ ID NO: 4 (LMVVGAVGV), SEQ ID NO: 7 (VVGAVGVGK), SEQ ID NO: 14 (GPVGVGKSV), SEQ ID NO: 69 (LMVVGAVGI), SEQ ID NO: 72 (LMVVGAVGL), SEQ ID NO: 131 (GAVGVGKSM), SEQ ID NO: 138 (GPVGVGKSA), SEQ ID NO: 142 (VTGAVGVGK), and SEQ ID NO: 198 (VAGAVGVGM). Two peptides, SEQ ID NO: 3 (GAVGVGKSL) and SEQ ID NO: 131 (GAVGVGKSM), are predicted to bind two of the HLA alleles with an affinity of 50 nM or less.

### MHC Class I Vaccine Design Procedure

In some embodiments, MHC class I vaccine design procedures consist of the following computations steps.

In some embodiments, the inputs for the computation are:
P_{1 . . . n}: Peptide sequence (length n) containing the neoantigen or pathogenic target(s) of interest (e.g., KRAS G12D, KRAS G12V, KRAS G12R, KRAS G12C, KRAS G13D). Pᵢ denotes the amino acid at position i.
t: Position of target mutation in P, t∈[1, . . . n](e.g., t=12 for KRAS G12D).
Σ₁: Threshold for potential presentation of peptides by MHC for peptide-MHC scoring (e.g., 500 nM binding affinity)
τ₂: Threshold for predicted display of peptides by MHC for peptide-MHC scoring (e.g., 50 nM binding affinity)
[symbol] : Set of HLA alleles (for HLA-A, HLA-B, HLA-C loci)
F: [symbol] ³→ [symbol] : Population haplotype frequencies (for OptiVax optimization and coverage evaluation).
N: Parameter for EvalVax and OptiVax objective function. Specifies minimum number of predicted per-individual hits for population coverage objective to consider the individual covered. Default=1 (computes P(n≥1) population coverage).

In some embodiments, Peptide-HLA Scoring Functions used are:
ScorePotential: P× [symbol] → [symbol] : Scoring function mapping a (peptide, HLA allele) pair to a prediction of peptide-HLA display. If predicted affinity ≤τ₁, then returns 1, else returns 0. Options include MHCflurry, NetMHCpan, PUFFIN, ensembles, or alternative metrics or software may be used, including models calibrated against immunogenicity data. [symbol]
SCOREDISPLAY: P× [symbol] → [symbol] : Scoring function mapping a (peptide, HLA allele) pair to a prediction of peptide-HLA display. If predicted affinity ≤τ₂, then returns 1, else returns 0. Options include MHCflurry, NetMHCpan, PUFFIN, ensembles, or alternative metrics or software may be used, including models calibrated against immunogenicity data.

Next, from the seed protein sequence (P), a set [symbol] of windowed native peptides spanning the protein sequence(s) is constructed. In some embodiments, 9-mers are produced, but this process can be performed with any desired window lengths and the resulting peptide sets combined.

$$𝒫 = \{ {P}_{j … j + 8} \vert j ∈ [ t - 8 , … , t ] , j \neq \{ t - 7 , t \} \}$$

The second condition j≠t −7, t}excludes peptides where the mutation at t is in positions P2 or P9 of the windowed 9-mer peptide (i.e., the anchor positions).

Next, each peptide sequence in [symbol] is scored against all HLA alleles in [symbol] for potential presentation using SCOREPOTENTIAL (with threshold τ₁=500 nM) and store results in a | [symbol] |×| [symbol] | matrix S:

$$S [ p , h ] = SCOREPOTENTIAL ( p , h ) ∀ p ∈ 𝒫 , h ∈ ℋ$$

Note that S is a binary matrix where 1 indicates the HLA is predicted to potentially present the peptide, and 0 indicates no potential presentation.
   Define base set of peptides B⊆ [symbol] :

$$B = \{ p ∈ 𝒫 \vert ∃ h s . t . S [ p , h ] = 1 \}$$

Thus, B contains the native peptides that are predicted to be potentially presented by at least 1 HLA.

Create a set of all heteroclitic peptides B′ stemming from peptides in B:

$${B}^{′} = {⋃}_{b ∈ B} ANCHOR - MODIFIED ( b )$$

where ANCHOR-MODIFIED(b) returns a set of all 399 anchor-modified peptides stemming from b (with all possible modifications to the amino acids at P2 and P9).

Next, all heteroclitic candidate peptides in B′ are scored against all HLA alleles in [symbol] for predicted display using SCOREDISPLAY (with threshold τ₂=50 nM), and store results in binary |B′|×| [symbol] |matrix S₁′:

$${S}_{1}^{′} [ {b}^{′} , h ] = SCOREDISPLAY ( {b}^{′} , h ) ∀ {b}^{′} ∈ {B}^{′} , h ∈ ℋ$$

Next, an updated scoring matrix

$${S}_{2}^{′}$$

is computed for heteroclitic peptides conditioned on the potential presentation of the corresponding base peptides by each HLA:

$${S}_{2}^{′} [ {b}^{′} , h ] = \{ \begin{matrix}{S}_{1}^{′} [ {b}^{′} , h ] , & if S [ b , h ] = 1 \\ 0 , & otherwise\end{matrix} ∀ {b}^{′} ∈ {B}^{′} , h ∈ ℋ$$

where each heteroclitic peptide b′∈B′ is a mutation of base peptide b∈B. This condition enforces that if h was not predicted to potentially present b, then all heteroclitic peptides b′ derived from b will not be displayed by h (even if h would otherwise be predicted to display b′).

In some embodiments, OptiVax-Robust is used to design a final peptide set from the union of base peptides and heteroclitic peptides B∪B′ (with corresponding scoring matrices S and

$${S}_{2}^{′}$$

for B and B′, respectively). Let [symbol] denote the compact set of vaccine peptides output by OptiVax containing k peptides. Note that [symbol] is not necessarily a superset of [symbol] . (In alternate embodiments, OptiVax can be used to augment the base set B with peptides from B′ using scoring matrix

$${S}_{2}^{′}$$

to return set [symbol] , and the final vaccine set [symbol] consists of peptides B∪ [symbol] ₖ.)

In some embodiments, this procedure is repeated independently for each target of interest, and the resulting independent vaccine sets can be merged into a combined vaccine as described below.

### MHC Class II Vaccine Design Procedure

In some embodiments, MHC class II vaccine design procedures consist of the following computations steps.

In some embodiments, the inputs for the computation are:
P_{1 . . . n}: Peptide sequence(s) (length n) containing the neoantigen(s) of interest (e.g., KRAS G12D, KRAS G12V, KRAS G12R, KRAS G12C, KRAS G13D). Pᵢ denotes the amino acid at position i.
t: Position of target mutation in P, t∈[1, . . . , n](e.g., t=12 for KRAS G12D).
τ₁: Threshold for potential presentation of peptides by MHC for peptide-MHC scoring (e.g., 500 nM binding affinity)
τ₂: Threshold for predicted display of peptides by MHC for peptide-MHC scoring (e.g., 50 nM binding affinity)
[symbol] : Set of HLA alleles (for HLA-DR, HLA-DQ, HLA-DP loci)
F: [symbol] ³→ [symbol] : Population haplotype frequencies (for OptiVax optimization and coverage evaluation).
N: Parameter for EvalVax and OptiVax objective function. Specifies minimum number of predicted per-individual hits for population coverage objective to consider the individual covered. Default=1 (computes P(n≥1) population coverage).

In some embodiments, Peptide-HLA Scoring Functions used are:
SCOREPOTENTIAL: P× [symbol] → [symbol] : Scoring function mapping a (peptide, HLA allele) pair to a prediction of peptide-HLA display. If predicted affinity ≤τ₁, then returns 1, else returns 0. Options include NetMHCIIpan, PUFFIN, ensembles, or alternative metrics or software may be used, including models calibrated against immunogenicity data.
SCOREDISPLAY: P× [symbol] → [symbol] : Scoring function mapping a (peptide, HLA allele) pair to a prediction of peptide-HLA display. If predicted affinity ≤τ₂, then returns 1, else returns 0. Options include NetMHCIIpan, PUFFIN, ensembles, or alternative metrics or software may be used, including models calibrated against immunogenicity data.
FindCore: P× [symbol] →[1, . . . , n]: Function mapping a (peptide, HLA allele) pair to a prediction of the 9-mer binding core. The core may be specified as the offset position (index) into the peptide where the core begins.

[symbol]

Next, from the seed protein sequence (P), a set of peptides spanning the protein sequence are constructed. Here, we extract all windowed peptides of length 13-25 spanning the target mutation, but this process can be performed using any desired window lengths (e.g., only 15-mers).

$$𝒫 = {⋃}_{k ∈ [ 13 , … , 25 ]} {𝒫}_{k}$$

$${𝒫}_{k} = \{ {P}_{j … j + ( k - 1 )} \vert j ∈ [ t - ( k - 1 ) , … , t ] \}$$

where [symbol] ₖ contains all sliding windows of length k, which are combined to form [symbol] . Note that here (unlike MHC class I), no peptides are excluded based on binding core or anchor residue positions (for MHC class II, filtering is performed in Paragraph 0063).

Next, each peptide sequence in P is scored against all HLA alleles in [symbol] for potential presentation using SCOREPOTENTIAL (with threshold τ₁=500 nM) and store results in a | [symbol] |×| [symbol] | matrix S₁:

$${S}_{1} [ p , h ] = SCOREPOT ENTIAL ( p , h ) ∀ p ∈ 𝒫 , h ∈ ℋ$$

Note that S₁ is a binary matrix where 1 indicates the HLA is predicted to potentially present the peptide, and 0 indicates no potential presentation.

For each (peptide, HLA allele) pair (p, h), identify/predict the 9-mer binding core using FINDCORE. The predicted binding core is recorded in a matrix C:

$$C [ p , h ] = FINDCORE ( p , h ) ∀ p ∈ 𝒫 , h ∈ ℋ$$

Next, an updated scoring matrix S₂ is computed for native peptides in [symbol] :

$${S}_{2} [ p , h ] = \{ \begin{matrix}{S}_{1} [ p , h ] , & if C [ p , h ] specifies {P}_{t} at a non - anchor position inside core \\ 0 , & otherwise\end{matrix} ∀ p ∈ 𝒫 , h ∈ ℋ$$

where Pₜ is the target residue of interest (e.g., the mutation site of KRAS G12D). This condition enforces the target residue to fall within the binding core at a non-anchor position for all (peptide, HLA allele) pairs with non-zero scores in S₂, and allows the binding core to vary by allele per peptide (as the binding cores of a particular peptide may differ based on the HLA allele presenting the peptide). Thus, for each pair (p, h), if the predicted binding core C[p, h] specifies the target residue Pₜ at an anchor position (P1, P4, P6, or P9 of the 9-mer core), or if Pₜ is not contained within the binding core, then S₂ [p, h]=0. In an alternate embodiment, Pₜ can be located outside of the core or inside the core in a non-anchor position.

Next, OptiVax-Robust is run with peptides [symbol] and scoring matrix S₂ to identify a non-redundant base set of peptides B⊆ [symbol] . (In alternate embodiments, B can be chosen as the entire set [symbol] rather than identifying a non-redundant base set.)

Next, a set of all heteroclitic peptides B′ is created stemming from peptides in B:

$${B}^{′} = {⋃}_{b ∈ ⋃ B} \{ ANCHOR - MODIFIED ( b , c ) ∀ c \vert ∃ h s . t . {S}_{2} [ b , h ] = 1 \}$$

where ANCHOR-MODIFIED(b,c) returns a set of all 20⁴−1 anchor-modified peptides stemming from b with all possible modifications to the amino acids at P1, P4, P6, and P9 of the 9-mer binding core c. Thus, for each base peptide b, the heteroclitic set B′ contains all anchor-modified peptides b′ with modifications to all unique cores of b identified for any HLA alleles that potentially present b with a valid core position as indicated by scoring matrix S₂.

Next, all heteroclitic candidate peptides in B′ are scored against all HLA alleles in [symbol] for predicted display using SCOREDISPLAY (with threshold τ₂=50 nM), and store results in binary |B′|×| [symbol] matrix S₁′:

$${S}_{1}^{′} [ {b}^{′} , h ] = ScoreDisplay ( {b}^{′} , h ) ∀ {b}^{′} ∈ {B}^{′} , h ∈ ℋ$$

For each (heteroclitic peptide, HLA allele) pair (b′,h), identify/predict the 9-mer binding core using FINDCORE. The predicted binding core is recorded in a matrix C′:

$${C}^{′} [ {b}^{′} , h ] = FINDCORE ( {b}^{′} , h ) ∀ {b}^{′} ∈ {B}^{′} , h ∈ ℋ$$

An updated scoring matrix

$${S}_{2}^{′}$$

is computed for heteroclitic peptides conditioned on the identified binding cores of a heteroclitic and base peptides occurring at the same offset by a particular HLA:

$${S}_{2}^{′} [ {b}^{′} , h ] = \{ \begin{matrix}{S}_{1}^{′} [ {b}^{′} , h ] , & if {C}^{′} [ {b}^{′} , h ] = C [ b , h ] \\ 0 , & otherwise\end{matrix} ∀ {b}^{′} ∈ {B}^{′} , h ∈ ℋ$$

where each heteroclitic peptide b′∈B′ is a mutation of base peptide b∈B. This condition enforces the binding core of the heteroclitic peptide b′ to be at the same relative position as the base peptide b, and, implicitly, enforces that the target residue Pₜ still falls in a non-anchor position within the 9-mer binding core (Step 3).

An updated scoring matrix

$${S}_{3}^{′}$$

is computed for heteroclitic peptides conditioned on the potential presentation of the corresponding base peptides by each HLA:

$${S}_{3}^{′} [ {b}^{′} , h ] = \{ \begin{matrix}{S}_{2}^{′} [ {b}^{′} , h ] , & if S [ b , h ] = 1 \\ 0 , & otherwise\end{matrix} ∀ {b}^{′} ∈ {B}^{′} , h ∈ ℋ$$

where each heteroclitic peptide b′∈B′ is a mutation of base peptide b∈B. This condition enforces that if h was not predicted to display b, then all heteroclitic peptides b′ derived from b will not be displayed by h (even if h would otherwise be predicted to display b′).

OptiVax-Robust is used to design a final peptide set from the union of base peptides and heteroclitic peptides B∪B′ (with corresponding scoring matrices S₂ and

$${S}_{3}^{′}$$

for B and B′, respectively). Let [symbol] denote the compact set of vaccine peptides output by OptiVax containing k peptides. Note that [symbol] is not necessarily a superset of [symbol] . (In alternate embodiments, OptiVax can be used to augment the base set B with peptides from B′ using scoring matrix

$${S}_{2}^{′}$$

to return set [symbol] ₖ, and the final vaccine set [symbol] consists of peptides B∪ [symbol] _{k′})

In some embodiments, this procedure is repeated independently for each single target of interest, and the resulting independent vaccine sets can be merged into a combined vaccine as described below.

### Methods for Combining Multiple Vaccines

The above described methods will produce an optimized foreign peptide set for one or more targets. In some embodiments, a method is provided for designing separate vaccines for MHC class I and class II based immunity for multiple targets (e.g., two or more targets such as KARS G12D and KRAS G12V).

In some embodiments, a method is disclosed for producing a combined peptide vaccine for multiple targets by using a table of presentations for a disease that is based upon empirical data from sources such as the Cancer Genome Atlas (TCGA). FIG. 5 shows one embodiment for factoring disease presentation type probabilities (pancreatic cancer, colon/rectum cancer, and bronchus/lung cancer) by probability, for each disease presentation, of target presented for various KRAS mutation targets (KRAS G12D, KRAS G12V, and KRAS G12R). A presentation is a unique set of targets that are presented by one form of a disease (e.g., distinct type of cancer as shown in FIG. 5). For each presentation, FIG. 5 shows an example of the probability of that presentation, and the probability that a given target is observed. For a given presentation, there can be one or more targets, each having a probability. In some embodiments, the method for multi-target vaccine design will allocate peptide resources for inducing disease immunity based on the presentation and respective target probabilities as shown in FIG. 5, for example. In some embodiments, presentations correspond to the prevalence of targets in different human populations or different risk groups. The probability of a target in a population is computed by summing for each possible presentation the probability of that presentation times the probability of the target in that presentation.

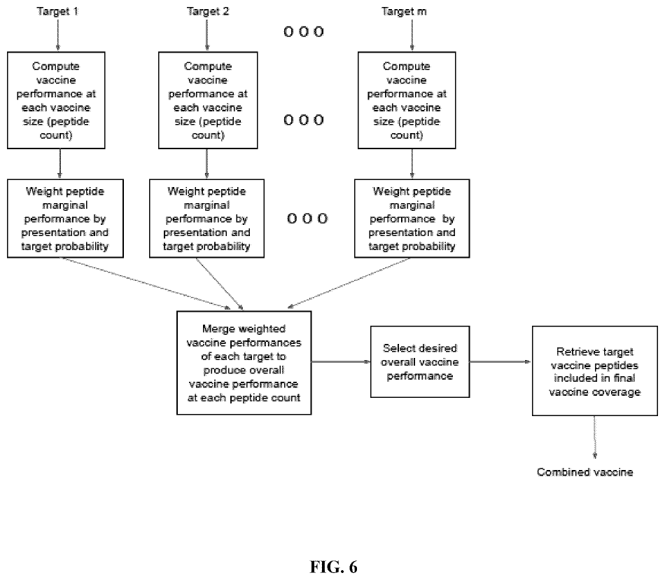

Referring to FIG. 6, in some embodiments, the method first includes designing an individual peptide vaccine for each target to create a combined vaccine design for multiple targets. This initially results in sets of target-specific vaccine designs. In some embodiments, the marginal vaccine performance of each target-specific vaccine at size k is defined by vaccine performance at size k minus the vaccine performance of the vaccine at size k minus one (see FIGS. 3-4). The composition of a vaccine may change as the number of peptides used in the vaccine increases, and thus for computing contributions to a combined vaccine the marginal vaccine performance of each target-specific vaccine is used instead of a specific set of peptides.

In some embodiments, the weighted marginal vaccine performance of a target-specific vaccine design for each target specific vaccine size is computed as shown in FIG. 6. For a given target specific vaccine size, its weighted vaccine performance is computed by multiplying its vaccine performance times the probability of the target in the population (e.g., by using values as shown in FIG. 5). The marginal weighted vaccine performance for a target specific vaccine is its weighted coverage at size k minus its coverage a size k minus one (e.g., see FIGS. 3-4). The marginal weighted vaccine performance of a target specific vaccine of size one is its weighted vaccine performance. The marginal weighted vaccine performances for all vaccines are combined into a single list, and the combined list is sorted from largest to least by the weighted marginal vaccine performances of the target specific vaccines as shown in FIG. 6. The combined vaccine of size n is then determined by the first n elements of this list. The peptides for the combined vaccine are determined by the individual peptide target vaccines whose sizes add to n and whose weighted vaccine performances sums to the same sum as the first n elements of the sorted list. This maximizes the vaccine performance of the combined vaccine of size n.

In some embodiments, the combined multiple target vaccine can be designed on its overall predicted coverage for the disease described depending on the presentation table used (e.g., see FIG. 5), by its predicted coverage for a specific indication, and/or by its predicted coverage for a specific target by adjusting the weighting used for vaccine performance accordingly. Once a desired level of coverage is selected, the peptides of the combined vaccine are determined by the contributions of target-specific designs. For example, if the combined vaccine includes a target-specific vaccine of size k, then the vaccine peptides for this target at size k are used in the combined vaccine.

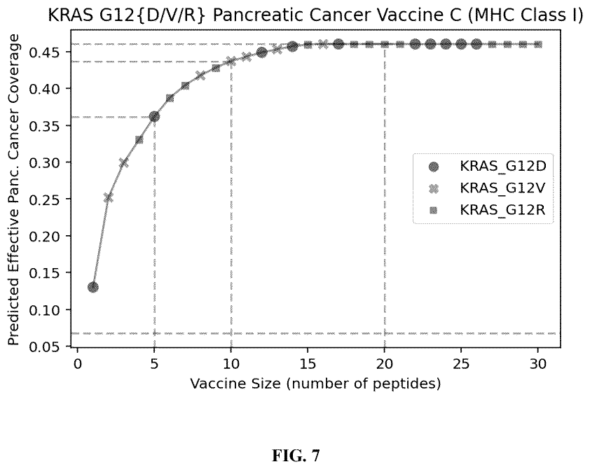

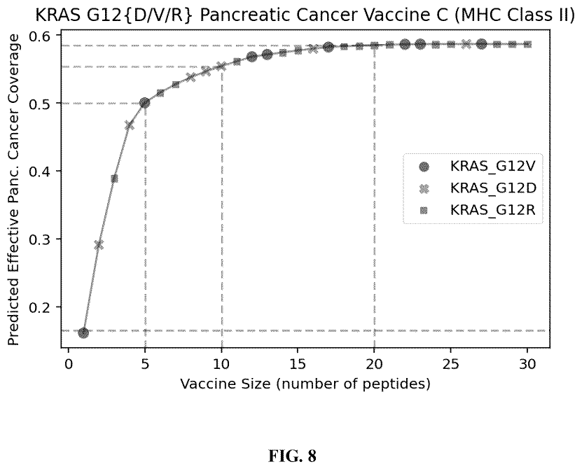

As an example of one embodiment, FIG. 5 shows three mutations (KRAS G12D, G12V, and G12R) and their respective probabilities of occurring in an individual with pancreatic cancer. FIG. 3 (MHC class I) and FIG. 4 (MHC class II) show the population coverage of target-specific vaccines for the KRAS G12D, G12V, G12R, G12C, and G13D targets using the methods for vaccines described herein. The marginal population coverage of each target-specific vaccine at a given vaccine size is the improvement in coverage at that size and the size less one. The coverage with no peptides is zero. The marginal coverage of each target-specific vaccine is multiplied by the probability of the target in the population as determined by the proportions as shown in FIG. 5 for the pancreas (pancreatic cancer). These weighted marginal coverages of all target-specific vaccines are sorted to determine the best target-specific compositions, and the resulting list describes the composition of a combined vaccine at each size k by taking the first k elements of the list. As an example of one embodiment, FIG. 7 (MHC Class I) and FIG. 8 (MHC Class II) show the target specific contributions at each vaccine size for a combined KRAS vaccine for the three mutations KRAS G12D, G12V, and G12R. The methods for combined vaccine protocol described herein was used to compute the examples in FIGS. 7 and 8. At each combined vaccine size, different components of the target-specific vaccines are utilized. Table 1 (below) contains the peptides present in independent (single target) and combined (multiple target) MHC class I vaccine designs for the KRAS G12D, G12V, G12R, G12C, and G13D targets. Table 2 (below) contains the contains the peptides present in independent (single target) MHC class II vaccine designs for the KRAS G12D, G12V, G12R, G12C, and G13D targets, and any subset of the individual/single target vaccines can be combined to create an MHC class II vaccine for two or more multiple targets. For alternate embodiments, Sequence Listing provides heteroclitic peptides useful in MHC class I vaccines for the KRAS G12D, G12V, G12R, G12C, and G13D targets.

### Combined Vaccine Design Procedure

In some embodiments, the procedure described herein is used to combine individual compact vaccines optimized for different targets into a single optimized combined vaccine.

In some embodiments, the computational inputs for the procedure are:
[symbol] : Set of neoantigen or pathogenic targets of interest (e.g., KRAS G12D, KRAS G12V, KRAS G12R)
[symbol] : Vaccine sets optimized individually for each target. Let [symbol] denote the optimal vaccine set of exactly k peptides for target t∈ [symbol] (e.g., as computed by the procedures describe above). Note that [symbol] may not necessarily be a superset of [symbol] .
W: [symbol] →[0,1]: Target weighting function mapping each target t∈ [symbol] to a probability or weight of t in a particular presentation of interest (e.g., pancreatic cancer; see Exhibit A, Table 1 for example).
POPULATIONCOVERAGE: [symbol] →[0,1]: Function mapping a peptide set into population coverage (e.g., EvalVax). This function may also take as input additional parameters, including HLA haplotype frequencies and a minimum per-individual number of peptide-HLA hits N (here, we compute coverage as P(n≥1) using EvalVax-Robust).

For each target t (individually) and vaccine size (peptide count) k, the unweighted population coverage c_{t,k} is computed:

$${c}_{t , k} = PopulationCoverage ( {𝒱}_{t , k} ) ∀ t ∈ 𝒯 , k$$

Note that for each target t, c_{t,k} is generally monotonically increasing and concave down for increasing values of k (each additional peptide increases coverage but with decreasing returns).

For each target t (individually), the marginal coverage m_{t,k} is computed of the k-th peptide added to the vaccine set:

$${m}_{t , k} = \{ \begin{matrix}{c}_{t , k} & if k = 1 \\ {c}_{t , k} - {c}_{t , k - 1} , & otherwise\end{matrix} ∀ t ∈ 𝒯 , k$$

Note that for each target t, m_{t,k} should be a monotonically decreasing function in k (by Step 1 above).

The weighted marginal population coverage \tilde{m}_{t,k} is computed using weights of each target in W:

$${\tilde{m}}_{t , k} = W ( t ) \cdot {m}_{t , k} ∀ t ∈ 𝒯 , k$$

The weighted marginal population coverage gives the effective marginal coverage of the k-th peptide in the vaccine weighted by the prevalence of the target in the presentation (by multiplication with the probability/weight of the target in the presentation).

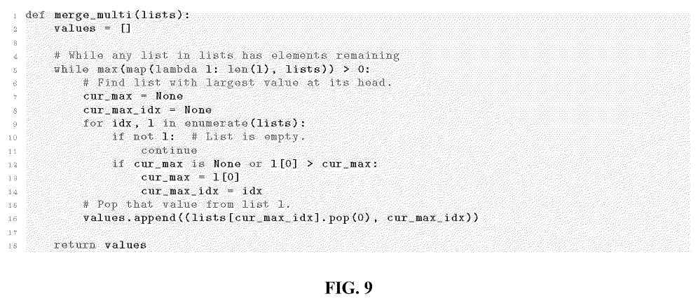

The individual vaccines are combined into a combined vaccine via the MERGEMULTI procedure called on the weighted marginal population coverage lists \tilde{m}ₜ=[\tilde{m}_{t,k}, k∈1, 2, . . . ]. FIG. 9 shows an example Python implementation of the MERGEMULTI function. This procedure takes as input multiple sorted (descending) lists and merges them into a single sorted (descending) list. Let M indicate the output of MERGEMULTI where each element Mk contains both the marginal weighted coverage and source (target) of the k-th peptide in the combined vaccine. The combined vaccine contains peptides from different targets. In particular, the combined vaccine with k peptides contains C_{t,k}=Σ_{j≤k} [symbol] {Mₖ from t}peptides from target t. Note that C_{t,k} ∈[0, . . . , k] and Σₜ C_{t,k}=k (C_{t,k} gives the distribution of the k peptides in the combined vaccine across the targets).

The optimal combined vaccine set [symbol] is defined as:

$${\hat{𝒱}}_{k} = {⋃}_{t ∈ 𝒯} {𝒱}_{t , {C}_{t , k}}$$

Thus, the combined vaccine with k peptides is the combination of the optimal individual (C_{t,k})-peptide vaccines. The marginal weighted coverage values of the combine vaccine Mk can be cumulatively summed over k to give the overall effective (target-weighted) population coverage of the combined vaccine containing k peptides as Σ_{j≤K} Mₖ (taking into account both the probabilities/weights of the targets in the presentation and the expected population coverage of peptides based on HLA display). The final vaccine size k can vary based upon the specific population coverage goals of the vaccine.

### MHC Class I Peptide Sequences

In some embodiments, a peptide vaccine (single target or combined multiple target vaccine) comprises about five, ten, or twenty MHC class I peptides with each peptide consisting of 8 or more amino acids. In some embodiments, an MHC class I peptide vaccine is intended for one or more of the KRAS G12D, G12V, and G12R targets. In some embodiments, the amino acid sequence of a first peptide in a five-peptide combined vaccine comprises SEQ ID NO: 1. GADGVGKSM (SEQ ID NO: 1). In some embodiments, the amino acid sequence of a second peptide in a five-peptide combined vaccine comprises SEQ ID NO: 2. LMVVGADGV (SEQ ID NO: 2). In some embodiments, the amino acid sequence of a third peptide in a five-peptide combined vaccine comprises SEQ ID NO: 3. GAVGVGKSL (SEQ ID NO: 3). In some embodiments, the amino acid sequence of a fourth peptide in a five-peptide combined vaccine comprises SEQ ID NO: 4. LMVVGAVGV (SEQ ID NO: 4). In some embodiments, the amino acid sequence of a fifth peptide in a five-peptide combined vaccine comprises SEQ ID NO: 5. VTGARGVGK (SEQ ID NO: 5). An example combined vaccine for the KRAS G12D, G12V, and G12R targets with five peptides (SEQ ID NO: 1 to SEQ ID NO: 5) is predicted to have a weighted population coverage of 0.3620.

In some embodiments, any one of the peptides (peptides 1-5) in the five-peptide vaccine comprise an amino acid sequence 80, 81, 82, 83, 84, 85, 86, 87, 88, 89, 90, 91, 92, 93, 94, 95, 96, 97, 98 or 99% identical to SEQ ID NO: 1, SEQ ID NO: 2, SEQ ID NO: 3, SEQ ID NO: 4, or SEQ ID NO: 5.

In some embodiments, the amino acid sequence of peptides 1 to 5 in a ten-peptide combined vaccine comprise SEQ ID NO: 1, SEQ ID NO: 2, SEQ ID NO: 3, SEQ ID NO: 4, and SEQ ID NO: 5. In some embodiments, the amino acid sequence of a sixth peptide in a ten-peptide combined vaccine comprises SEQ ID NO: 6. VMGAVGVGK (SEQ ID NO: 6). In some embodiments, the amino acid sequence of a seventh peptide in a ten-peptide combined vaccine comprises SEQ ID NO: 7. VVGAVGVGK (SEQ ID NO: 7). In some embodiments, the amino acid sequence of an eight peptide in a ten-peptide combined vaccine comprises SEQ ID NO: 8. GARGVGKSY (SEQ ID NO: 8). In some embodiments, the amino acid sequence of a ninth peptide in a ten-peptide combined vaccine comprises SEQ ID NO: 9. GPRGVGKSA (SEQ ID NO: 9). In some embodiments, the amino acid sequence of a tenth peptide in a ten-peptide combined vaccine comprises SEQ ID NO: 10. LMVVGARGV (SEQ ID NO: 10). An example combined vaccine for the KRAS G12D, G12V, and G12R targets with ten peptides (SEQ ID NO: 1 to SEQ ID NO: 10) is predicted to have a weighted population coverage of 0.4374.

In some embodiments, any one of the peptides (peptides 1-10) in the ten-peptide vaccine comprise an amino acid sequence 80, 81, 82, 83, 84, 85, 86, 87, 88, 89, 90, 91, 92, 93, 94, 95, 96, 97, 98 or 99% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 10.

In some embodiments, the amino acid sequence of peptides 1 to 10 in a twenty-peptide combined vaccine comprise SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. In some embodiments, the amino acid sequence of an 11^{th} peptide in a twenty-peptide combined vaccine comprises SEQ ID NO: 11. GADGVGKSL (SEQ ID NO: 11). In some embodiments, the amino acid sequence of a 12^{th} peptide in a twenty-peptide combined vaccine comprises SEQ ID NO: 12. GADGVGKSY (SEQ ID NO: 12). In some embodiments, the amino acid sequence of a 13^{th} peptide in a twenty-peptide combined vaccine comprises SEQ ID NO: 13. GYDGVGKSM (SEQ ID NO: 13). In some embodiments, the amino acid sequence of a 14^{th} peptide in a twenty-peptide combined vaccine comprises SEQ ID NO: 14. GPVGVGKSV (SEQ ID NO: 14). In some embodiments, the amino acid sequence of a 15^{th} peptide in a twenty-peptide combined vaccine comprises SEQ ID NO: 15. LTVVGAVGV (SEQ ID NO: 15). In some embodiments, the amino acid sequence of a 16^{th} peptide in a twenty-peptide combined vaccine comprises SEQ ID NO: 16. VVGAVGVGR (SEQ ID NO: 16). In some embodiments, the amino acid sequence of a 17^{th} peptide in a twenty-peptide combined vaccine comprises SEQ ID NO: 17. GARGVGKSM (SEQ ID NO: 17). In some embodiments, the amino acid sequence of an 18^{th} peptide in a twenty-peptide combined vaccine comprises SEQ ID NO: 18. GPRGVGKSV (SEQ ID NO: 18). In some embodiments, the amino acid sequence of a 19^{th} peptide in a twenty-peptide combined vaccine comprises SEQ ID NO: 19. LLVVGARGV (SEQ ID NO: 19). In some embodiments, the amino acid sequence of a 20^{th} peptide in a twenty-peptide combined vaccine comprises SEQ ID NO: 20. VAGARGVGM (SEQ ID NO: 20). An example combined vaccine for the KRAS G12D, G12V, and G12R targets with twenty peptides (SEQ ID NO: 1 to SEQ ID NO: 20) is predicted to have a weighted population coverage of 0.4604.

In some embodiments, any one of the peptides (peptides 1-20) in the twenty-peptide vaccine comprise an amino acid sequence 80, 81, 82, 83, 84, 85, 86, 87, 88, 89, 90, 91, 92, 93, 94, 95, 96, 97, 98 or 99% identical to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20

Table 1 shows MHC class I peptide sequences described herein including the respective SEQ ID NO, amino acid sequence corresponding to the SEQ ID NO, KRAS protein target (with specific mutation), the seed amino acid sequence (i.e., the amino acid sequence of the wild type KRAS fragment), the amino acid substitution (if any) for heteroclitic peptides at positions 2 and 9, and notes detailing embodiments in which the peptide may be included in a 5, 10, or 20 combined peptide vaccine as described herein. Table 1 also includes additional peptide sequences comprising SEQ ID NOs: 21-41. In some embodiments, any combination of peptides listed in Table 1 (SEQ ID NOs: 1-41) may be used to create a combined peptide vaccine having between about 2 and about 40 peptides. In some embodiments, any one of the peptides (peptides 1-41; SEQ ID NOs: 1-41) in the combined vaccine comprises an amino acid sequence 80, 81, 82, 83, 84, 85, 86, 87, 88, 89, 90, 91, 92, 93, 94, 95, 96, 97, 98 or 99% identical to any of SEQ ID NOs: 1-41.

TABLE 1
Example KRAS Vaccine Peptides (MHC class I)

| SEQ ID | Sequence corresponding | | | Heteroclitic | Heteroclitic | |
|---|---|---|---|---|---|---|
| NO | to SEQ ID | Target | Seed | Modification P2 | Modification P9 | Note |
| SEQ ID | GADGVGKSM | KRAS | GADGVGKSA | — | A9M | Individual KRAS |
| NO: 1 | | G12D | | | | G12D (MHCflurry); |
| | | | | | | Combined (5 peptide) |
| | | | | | | (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | LMVVGADGV | KRAS | LVVVGADGV | V2M | — | Individual KRAS |
| NO: 2 | | G12D | | | | G12D (MHCflurry); |
| | | | | | | Individual KRAS |
| | | | | | | G12D (NetMHCpan); |
| | | | | | | Combined (5 peptide) |
| | | | | | | (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | GAVGVGKSL | KRAS | GAVGVGKSA | — | A9L | Individual KRAS |
| NO: 3 | | G12V | | | | G12V (MHCflurry); |
| | | | | | | Combined (5 peptide) |
| | | | | | | (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | LMVVGAVGV | KRAS | LVVVGAVGV | V2M | — | Individual KRAS |
| NO: 4 | | G12V | | | | G12V (MHCflurry); |
| | | | | | | Individual KRAS |
| | | | | | | G12V (NetMHCpan); |
| | | | | | | Combined (5 peptide) |
| | | | | | | (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | VTGARGVGK | KRAS | VVGARGVGK | V2T | — | Individual KRAS |
| NO: 5 | | G12R | | | | G12R (MHCflurry); |
| | | | | | | Combined (5 peptide) |
| | | | | | | (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | VMGAVGVGK | KRAS | VVGAVGVGK | V2M | — | Individual KRAS |
| NO: 6 | | G12V | | | | G12V (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | VVGAVGVGK | KRAS | VVGAVGVGK | — | — | Individual KRAS |
| NO: 7 | | G12V | | | | G12V (MHCflurry); |
| | | | | | | Individual KRAS |
| | | | | | | G12V (NetMHCpan); |
| | | | | | | Combined (10 |
| | | | | | | peptide) (MHCflurry); |
| | | | | | | Combined (20 |
| | | | | | | peptide) (MHCflurry) |
| SEQ ID | GARGVGKSY | KRAS | GARGVGKSA | — | A9Y | Individual KRAS |
| NO: 8 | | G12R | | | | G12R (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | GPRGVGKSA | KRAS | GARGVGKSA | A2P | — | Individual KRAS |
| NO: 9 | | G12R | | | | G12R (MHCflurry); |
| | | | | | | Combined (10 |
| | | | | | | peptide) (MHCflurry); |
| | | | | | | Combined (20 |
| | | | | | | peptide) (MHCflurry) |
| SEQ ID | LMVVGARGV | KRAS | LVVVGARGV | V2M | — | Individual KRAS |
| NO: 10 | | G12R | | | | G12R (MHCflurry); |
| | | | | | | Individual KRAS |
| | | | | | | G12R (NetMHCpan); |
| | | | | | | Combined (10 |
| | | | | | | peptide) (MHCflurry); |
| | | | | | | Combined (20 |
| | | | | | | peptide) (MHCflurry) |
| SEQ ID | GADGVGKSL | KRAS | GADGVGKSA | — | A9L | Individual KRAS |
| NO: 11 | | G12D | | | | G12D (MHCflurry); |
| | | | | | | Combined (20 |
| | | | | | | peptide) (MHCflurry) |
| SEQ ID | GADGVGKSY | KRAS | GADGVGKSA | — | A9Y | Individual KRAS |
| NO: 12 | | G12D | | | | G12D (MHCflurry); |
| | | | | | | Combined (20 |
| | | | | | | peptide) (MHCflurry) |
| SEQ ID | GYDGVGKSM | KRAS | GADGVGKSA | A2Y | A9M | Individual KRAS |
| NO: 13 | | G12D | | | | G12D (MHCflurry); |
| | | | | | | Combined (20 |
| | | | | | | peptide) (MHCflurry) |
| SEQ ID | GPVGVGKSV | KRAS | GAVGVGKSA | A2P | A9V | Combined (20 |
| NO: 14 | | G12V | | | | peptide) (MHCflurry) |
| SEQ ID | LTVVGAVGV | KRAS | LVVVGAVGV | V2T | — | Individual KRAS |
| NO: 15 | | G12V | | | | G12V (NetMHCpan); |
| | | | | | | Combined (20 |
| | | | | | | peptide) (MHCflurry) |
| SEQ ID | VVGAVGVGR | KRAS | VVGAVGVGK | — | K9R | Individual KRAS |
| NO: 16 | | G12V | | | | G12V (MHCflurry); |
| | | | | | | Individual KRAS |
| | | | | | | G12V (NetMHCpan); |
| | | | | | | Combined (20 |
| | | | | | | peptide) (MHCflurry) |
| SEQ ID | GARGVGKSM | KRAS | GARGVGKSA | — | A9M | Combined (20 |
| NO: 17 | | G12R | | | | peptide) (MHCflurry) |
| SEQ ID | GPRGVGKSV | KRAS | GARGVGKSA | A2P | A9V | Combined (20 |
| NO: 18 | | G12R | | | | peptide) (MHCflurry) |
| SEQ ID | LLVVGARGV | KRAS | LVVVGARGV | V2L | — | Individual KRAS |
| NO: 19 | | G12R | | | | G12R (NetMHCpan); |
| | | | | | | Combined (20 |
| | | | | | | peptide) (MHCflurry) |
| SEQ ID | VAGARGVGM | KRAS | VVGARGVGK | V2A | K9M | Individual KRAS |
| NO: 20 | | G12R | | | | G12R (MHCflurry); |
| | | | | | | Combined (20 |
| | | | | | | peptide) (MHCflurry) |
| SEQ ID | LTVVGADGV | KRAS | LVVVGADGV | V2T | — | Individual KRAS |
| NO: 21 | | G12D | | | | G12D (NetMHCpan) |
| SEQ ID | LLVVGADGV | KRAS | LVVVGADGV | V2L | — | Individual KRAS |
| NO: 22 | | G12D | | | | G12D (NetMHCpan) |
| SEQ ID | LMVVGADGL | KRAS | LVVVGADGV | V2M | V9L | Individual KRAS |
| NO: 23 | | G12D | | | | G12D (NetMHCpan) |
| SEQ ID | VMGAVGVGR | KRAS | VVGAVGVGK | V2M | K9R | Individual KRAS |
| NO: 24 | | G12V | | | | G12V (NetMHCpan) |
| SEQ ID | VMGARGVGK | KRAS | VVGARGVGK | V2M | — | Individual KRAS |
| NO: 25 | | G12R | | | | G12R (NetMHCpan) |
| SEQ ID | GACGVGKSL | KRAS | GACGVGKSA | — | A9L | Individual KRAS |
| NO: 26 | | G12C | | | | G12C (MHCflurry) |
| SEQ ID | LMVVGACGV | KRAS | LVVVGACGV | V2M | — | Individual KRAS |
| NO: 27 | | G12C | | | | G12C (MHCflurry); |
| | | | | | | Individual KRAS |
| | | | | | | G12C (NetMHCpan) |
| SEQ ID | LTVVGACGV | KRAS | LVVVGACGV | V2T | — | Individual KRAS |
| NO: 28 | | G12C | | | | G12C (MHCflurry); |
| | | | | | | Individual KRAS |
| | | | | | | G12C (NetMHCpan) |
| SEQ ID | VTGACGVGK | KRAS | VVGACGVGK | V2T | — | Individual KRAS |
| NO: 29 | | G12C | | | | G12C (MHCflurry) |
| SEQ ID | VVGACGVGR | KRAS | VVGACGVGK | — | K9R | Individual KRAS |
| NO: 30 | | G12C | | | | G12C (MHCflurry) |
| SEQ ID | AADVGKSAM | KRAS | AGDVGKSAL | G2A | L9M | Individual KRAS |
| NO: 31 | | G13D | | | | G13D (MHCflurry); |
| | | | | | | Individual KRAS |
| | | | | | | G13D (NetMHCpan) |
| SEQ ID | AEDVGKSAM | KRAS | AGDVGKSAL | G2E | L9M | Individual KRAS |
| NO: 32 | | G13D | | | | G13D (MHCfluriy) |
| SEQ ID | AYDVGKSAM | KRAS | AGDVGKSAL | G2Y | L9M | Individual KRAS |
| NO: 33 | | G13D | | | | G13D (MHCfluriy) |
| SEQ ID | DAGKSALTV | KRAS | DVGKSALTI | V2A | I9V | Individual KRAS |
| NO: 34 | | G13D | | | | G13D (MHCflurry) |
| SEQ ID | GAGDVGKSM | KRAS | GAGDVGKSA | — | A9M | Individual KRAS |
| NO: 35 | | G13D | | | | G13D (MHCfluriy) |
| SEQ ID | LQVVGACGV | KRAS | LVVVGACGV | V2Q | — | Individual KRAS |
| NO: 36 | | G12C | | | | G12C (NetMHCpan) |
| SEQ ID | VMGACGVGK | KRAS | VVGACGVGK | V2M | — | Individual KRAS |
| NO: 37 | | G12C | | | | G12C (NetMHCpan) |
| SEQ ID | VMGACGVGR | KRAS | VVGACGVGK | V2M | K9R | Individual KRAS |
| NO: 38 | | G12C | | | | G12C (NetMHCpan) |
| SEQ ID | AADVGKSAL | KRAS | AGDVGKSAL | G2A | — | Individual KRAS |
| NO: 39 | | G13D | | | | G13D (NetMHCpan) |
| SEQ ID | ASDVGKSAL | KRAS | AGDVGKSAL | G2S | — | Individual KRAS |
| NO: 40 | | G13D | | | | G13D (NetMHCpan) |
| SEQ ID | ASDVGKSAM | KRAS | AGDVGKSAL | G2S | L9M | Individual KRAS |
| NO: 41 | | G13D | | | | G13D (NetMHCpan) |

Additional amino acid sequences of MHC class I heteroclitic peptides are provided in Sequence Listings (SEQ ID NOs: 67-1522). In some embodiments, any combination of MHC class I peptides disclosed herein (SEQ ID NOs: 1-41 and 67-1522) may be used to create a combined peptide vaccine having between about 2 and about 40 peptides. In some embodiments, any one of the peptides (SEQ ID NOs: 1-41 and 67-1522) in the combined vaccine comprises or contains an amino acid sequence 80, 81, 82, 83, 84, 85, 86, 87, 88, 89, 90, 91, 92, 93, 94, 95, 96, 97, 98 or 99% identical to any of SEQ ID NOs: 1-41 or 67-1522.

### MHC Class II Peptide Sequences

In some embodiments, a peptide vaccine (single target or combined multiple target vaccine) comprises about 2 to 40 MHC class II peptides with each peptide consisting of about 20 amino acids. In some embodiments, an MHC class II peptide vaccine is intended for one or more of the KRAS G121D, G12V, G12R, G12C, and G13D targets.

Table 2 summarizes MHC class II peptide sequences described herein including the respective SEQ ID NO, amino acid sequence corresponding to the SEQ ID NO, the amino acid sequence corresponding to the peptide's binding core, the KRAS protein target (with specific mutation), the seed amino acid sequence (i.e., the amino acid sequence of the wild type KRAS fragment), the seed amino acid sequence of the binding core, and the amino acid substitution (if any) for heteroclitic peptides at positions 1, 4, 6, and 9. Table 2 includes peptide sequences comprising SEQ ID NOs: 42-66. SEQ ID NOs: 42-65 (Table 2) encode for recombinant peptides. In some embodiments, any combination ofpeptides listed in Table 2 (SEQ ID NOs: 42-66) may be used to create a single target (individual) or combined peptide vaccine having between about 2 and about 40 peptides. In some embodiments, any one of the peptides (peptides 42-66; SEQ ID NOs: 42-66) in the combined vaccine comprises an amino acid sequence 80, 81, 82, 83, 84, 85, 86, 87, 88, 89, 90, 91, 92, 93, 94, 95, 96, 97, 98 or 99% identical to any of SEQ ID NOs: 42-66.

TABLE 2
Example KRAS Vaccine Peptides (MHC class II)

| | | | | | | Hetero- | Hetero- | Hetero- | Hetero- | |
|---|---|---|---|---|---|---|---|---|---|---|
| | | | | | | clitic | clitic | clitic | clitic | |
| | Sequence | | | | | Modifi- | Modifi- | Modifi- | Modifi- | |
| SEQ | corresponding | | | | | cation | cation | cation | cation | |
| ID NO | to SEQ ID | Core | Target | Seed | Seed Core | P1 | P4 | P6 | P9 | Note |
| SEQ | EYKFVVFGSDGAG | FVVFG | KRAS | EYKLVVVGADGVG | LVVVGADGV | L1F | V4F | A6S | V9A | Individual |
| ID | KS | SDGA | G12D | KS | | | | | | KRAS |
| NO: | | | | | | | | | | G12D |
| 42 | | | | | | | | | | (NetMHCIIpan) |
| SEQ | EYKFVVIGNDGAG | FVVIG | KRAS | EYKLVVVGADGVG | LVVVGADGV | L1F | V4I | A6N | V9A | Individual |
| ID | KSALTIQLIQN | NDGA | G12D | KSALTIQLIQN | | | | | | KRAS |
| NO: | | | | | | | | | | G12D |
| 43 | | | | | | | | | | (NetMHCIIpan) |
| SEQ | EYKFVVLGADGAG | FVVLG | KRAS | EYKLVVVGADGVG | LVVVGADGV | L1F | V4L | — | V9A | Individual |
| ID | KS | ADGA | G12D | KS | | | | | | KRAS |
| NO: | | | | | | | | | | G12D |
| 44 | | | | | | | | | | (NetMHCIIpan) |
| SEQ | MTEYKFVVSGADG | FVVSG | KRAS | MTEYKLVVVGADG | LVVVGADGV | L1F | V4S | — | V9I | Individual |
| ID | IGKSALT | ADGI | G12D | VGKSALT | | | | | | KRAS |
| NO: | | | | | | | | | | G12D |
| 45 | | | | | | | | | | (NetMHCIIpan) |
| SEQ | MTEYKFVVYGSDG | FVVYG | KRAS | MTEYKLVVVGADG | LVVVGADGV | L1F | V4Y | A6S | V9I | Individual |
| ID | IGKSALT | SDGI | G12D | VGKSALT | | | | | | KRAS |
| NO: | | | | | | | | | | G12D |
| 46 | | | | | | | | | | (NetMHCIIpan) |
| SEQ | EYKFVVIGRVGHG | FVVIG | KRAS | EYKLVVVGAVGVG | LVVVGAVGV | L1F | V4I | A6R | V9H | Individual |
| ID | KS | RVGH | G12V | KS | | | | | | KRAS |
| NO: | | | | | | | | | | G12V |
| 47 | | | | | | | | | | (NetMHCIIpan) |
| SEQ | EYKFVVLGTVGHG | FVVLG | KRAS | EYKLVVVGAVGVG | LVVVGAVGV | L1F | V4L | A6T | V9H | Individual |
| ID | KS | TVGH | G12V | KS | | | | | | KRAS |
| NO: | | | | | | | | | | G12V |
| 48 | | | | | | | | | | (NetMHCIIpan) |
| SEQ | EYKFVVYGNVGM | FVVYG | KRAS | EYKLVVVGAVGVG | LVVVGAVGV | L1F | V4Y | A6N | V9M | Individual |
| ID | GKS | NVGM | G12V | KS | | | | | | KRAS |
| NO: | | | | | | | | | | G12V |
| 49 | | | | | | | | | | (NetMHCIIpan) |
| SEQ | EYKIVVAGNVGIGK | IVVAG | KRAS | EYKLVVVGAVGVG | LVVVGAVGV | L1I | V4A | A6N | V9I | Individual |
| ID | S | NVGI | G12V | KS | | | | | | KRAS |
| NO: | | | | | | | | | | G12V |
| 50 | | | | | | | | | | (NetMHCIIpan) |
| SEQ | TEYKIVVMGNVGY | IVVMG | KRAS | TEYKLVVVGAVGV | LVVVGAVGV | L1I | V4M | A6N | V9Y | Individual |
| ID | GK | NVGY | G12V | GK | | | | | | KRAS |
| NO: | | | | | | | | | | G12V |
| 51 | | | | | | | | | | (NetMHCIIpan) |
| SEQ | MTEYKFVVFGSRG | FVVFG | KRAS | MTEYKLVVVGARG | LVVVGARGV | L1F | V4F | A6S | — | Individual |
| ID | VGKSALT | SRGV | G12R | VGKSALT | | | | | | KRAS |
| NO: | | | | | | | | | | G12R |
| 52 | | | | | | | | | | (NetMHCIIpan) |
| SEQ | MTEYKFVVIGNRG | FVVIG | KRAS | MTEYKLVVVGARG | LVVVGARGV | L1F | V4I | A6N | — | Individual |
| ID | VGKSALT | NRGV | G12R | VGKSALT | | | | | | KRAS |
| NO: | | | | | | | | | | G12R |
| 53 | | | | | | | | | | (NetMHCIIpan) |
| SEQ | MTEYKFVVIGVRG | FVVIG | KRAS | MTEYKLVVVGARG | LVVVGARGV | L1F | V4I | A6V | V9D | Individual |
| ID | DGKSALT | VRGD | G12R | VGKSALT | | | | | | KRAS |
| NO: | | | | | | | | | | G12R |
| 54 | | | | | | | | | | (NetMHCIIpan) |
| SEQ | MTEYKFVVMGSRG | FVVM | KRAS | MTEYKLVVVGARG | LVVVGARGV | L1F | V4M | A6S | V9A | Individual |
| ID | AGKSALT | GSRGA | G12R | VGKSALT | | | | | | KRAS |
| NO: | | | | | | | | | | G12R |
| 55 | | | | | | | | | | (NetMHCIIpan) |
| SEQ | VVVIARGVPKSLLT | IARGV | KRAS | VVVGARGVGKSAL | GARGVGKSA | G1I | — | G6P | A9L | Individual |
| ID | I | PKSL | G12R | TI | | | | | | KRAS |
| NO: | | | | | | | | | | G12R |
| 56 | | | | | | | | | | (NetMHCIIpan) |
| SEQ | EYKFVVFGNCGAG | FVVFG | KRAS | EYKLVVVGACGVG | LVVVGACGV | L1F | V4F | A6N | V9A | Individual |
| ID | KS | NCGA | G12C | KS | | | | | | KRAS |
| NO: | | | | | | | | | | G12C |
| 57 | | | | | | | | | | (NetMHCIIpan) |
| SEQ | EYKFVVSGACGVG | FVVSG | KRAS | EYKLVVVGACGVG | LVVVGACGV | L1F | V4S | — | — | Individual |
| ID | KS | ACGV | G12C | KS | | | | | | KRAS |
| NO: | | | | | | | | | | G12C |
| 58 | | | | | | | | | | (NetMHCIIpan) |
| SEQ | EYKFVVSGNCGLG | FVVSG | KRAS | EYKLVVVGACGVG | LVVVGACGV | L1F | V4S | A6N | V9L | Individual |
| ID | KS | NCGL | G12C | KS | | | | | | KRAS |
| NO: | | | | | | | | | | G12C |
| 59 | | | | | | | | | | (NetMHCIIpan) |
| SEQ | EYKLVVMGPCGAG | LVVM | KRAS | EYKLVVVGACGVG | LVVVGACGV | — | V4M | A6P | V9A | Individual |
| ID | KS | GPCGA | G12C | KS | | | | | | KRAS |
| NO: | | | | | | | | | | G12C |
| 60 | | | | | | | | | | (NetMHCIIpan) |
| SEQ | KLVIVGICKVGHSA | IVGICK | KRAS | KLVVVGACGVGKS | VVGACGVGK | V1I | A4I | G6K | K9H | Individual |
| ID | L | VGH | G12C | AL | | | | | | KRAS |
| NO: | | | | | | | | | | G12C |
| 61 | | | | | | | | | | (NetMHCIIpan) |
| SEQ | EYKFVVFGNGDLG | FVVFG | KRAS | EYKLVVVGAGDVG | LVVVGAGDV | L1F | V4F | A6N | V9L | Individual |
| ID | KS | NGDL | G13D | KS | | | | | | KRAS |
| NO: | | | | | | | | | | G13D |
| 62 | | | | | | | | | | (NetMHCIIpan) |
| SEQ | EYKFVVMGNGDSG | FVVM | KRAS | EYKLVVVGAGDVG | LVVVGAGDV | L1F | V4M | A6N | V9S | Individual |
| ID | KS | GNGDS | G13D | KS | | | | | | KRAS |
| NO: | | | | | | | | | | G13D |
| 63 | | | | | | | | | | (NetMHCIIpan) |
| SEQ | EYKFVVSGSGDVG | FVVSG | KRAS | EYKLVVVGAGDVG | LVVVGAGDV | L1F | V4S | A6S | — | Individual |
| ID | KS | SGDV | G13D | KS | | | | | | KRAS |
| NO: | | | | | | | | | | G13D |
| 64 | | | | | | | | | | (NetMHCIIpan) |
| SEQ | EYKIVVMGRGDMG | IVVMG | KRAS | EYKLVVVGAGDVG | LVVVGAGDV | L1I | V4M | A6R | V9M | Individual |
| ID | KS | RGDM | G13D | KS | | | | | | KRAS |
| NO: | | | | | | | | | | G13D |
| 65 | | | | | | | | | | (NetMHCIIpan) |
| SEQ | YKLVVVGAGDVG | — | KRAS | — | — | — | — | — | — | Individual |
| ID | KSA | | G13D | | | | | | | KRAS |
| NO: | | | | | | | | | | G13D |
| 66 | | | | | | | | | | (NetMHCIIpan) |

In some embodiments, any combination of MHC class I and/or MHC class II peptides disclosed herein (SEQ ID NOs: 1-1522) may be used to create a single target (individual) or combined peptide vaccine having between about 2 and about 40 peptides. In some embodiments, any one of the peptides (peptides 1-1522; SEQ ID NOs: 1-1522) in the combined vaccine comprises an amino acid sequence 80, 81, 82, 83, 84, 85, 86, 87, 88, 89, 90, 91, 92, 93, 94, 95, 96, 97, 98 or 99% identical to any of SEQ ID NOs: 1-1522.

mRNA and DNA Vaccines

In some embodiments, vaccine peptides are encoded as mRNA or DNA molecules and are administered for expression in vivo as is known in the art. One example of the delivery of vaccines by mRNA is found in Kranz et al. (2016), incorporated herein by reference. In one embodiment, a construct comprises 10 peptides, including a five-peptide MHC class I combined pancreatic cancer vaccine (targets: KRAS G12D, G12V, G12R) and a five-peptide MHC class II combined pancreatic cancer vaccine (targets: KRAS G12D, G12V, G12R), as optimized by the procedure described herein. Peptides are prepended with a secretion signal sequence at the N-terminus and followed by an MHC class I trafficking signal (MITD) (Kreiter et al., 2008; Sahin et al., 2017). The MITD has been shown to route antigens to pathways for HLA class I and class II presentation (Kreiter et al., 2008). Here we combine all peptides of each MHC class into a single construct using non-immunogenic glycine/serine linkers from Sahin et al. (2017), though it is also plausible to construct individual constructs containing single peptides with the same secretion and MITD signals as demonstrated by Kreiter et al. (2008).

In some embodiments, the amino acid sequence encoded by the mRNA vaccine comprises SEQ ID NO: 1523. Underlined amino acids correspond to the signal peptide (or leader) sequence. Bolded amino acids correspond to MHC class I (9 amino acids in length; 5 peptides) and MHC class II (13-25 amino acids in length; 5 peptides) peptide sequences. Italicized amino acids correspond to the trafficking signal.

(SEQ ID NO: 1523)
MRVTAPRTLILLLSGALALTETWAGSGGSGGGGSGGGADGVGKSMGGSGG
GGSGGLMVVGADGVGGSGGGGSGGGAVGVGKSLGGSGGGGSGGLMVVGAV
GVGGSGGGGSGGVTGARGVGKGGSGGGGSGGEYKFVVLGTVGHGKSGGSG
GGGSGGEYKIVVAGNVGIGKSGGSGGGGSGGEYKFVVFGSDGAGKSGGSG
GGGSGGMTEYKFVVSGADGIGKSALTGGSGGGGSGGMTEYKFVVIGNRGV
GKSALTGGSLGGGGSGIVGIVAGLAVLAVVVIGAVVATVMCRRKSSGGKG
GSYSQAASSDSAQGSDVSLTA.

In some embodiments, the vaccine is an mRNA vaccine comprising a nucleic acids sequence encoding the amino acid sequence consisting of SEQ ID NO: 1523. In some embodiments, the nucleic acid sequence of the mRNA vaccine encodes for an amino acid sequence 80, 81, 82, 83, 84, 85, 86, 87, 88, 89, 90, 91, 92, 93, 94, 95, 96, 97, 98 or 99% identical to SEQ ID NO: 1523.

In some embodiments, the vaccine is a DNA vaccine comprising a nucleic acids sequence encoding the amino acid sequence consisting of SEQ ID NO: 1523. In some embodiments, the nucleic acid sequence of the DNA vaccine encodes for an amino acid sequence 80, 81, 82, 83, 84, 85, 86, 87, 88, 89, 90, 91, 92, 93, 94, 95, 96, 97, 98 or 99% identical to SEQ ID NO: 1523.

In some embodiments, one or more MHC class I and/or MHC class II peptides disclosed herein (SEQ ID NO: 1-1522) can be encoded in one or more mRNA or DNA molecules and administered for expression in vivo. In some embodiments between about 2 and about 40 peptide sequences are encoded in one or more mRNA constructs. In some embodiments, between about 2 and about 40 peptide sequences are encoded in one or more DNA constructs (i.e., nucleic acids encoding the amino acids sequences comprising on or more of SEQ ID NOs: 1-1522). In some embodiments, the amino acid sequence of the mRNA vaccine or the nucleic acid sequence of the DNA vaccine encodes for an amino acid sequence 80, 81, 82, 83, 84, 85, 86, 87, 88, 89, 90, 91, 92, 93, 94, 95, 96, 97, 98 or 99% identical to any of SEQ ID NOs: 1-1522.

### Non-Limiting Embodiments of the Subject Matter

In one aspect, the invention provides for a nucleic acid sequence encoding two or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In some embodiments, the nucleic acid sequence is an immunogenic composition. In some embodiments, the nucleic acid sequence is administered in a construct for expression in vivo. In some embodiments, the in vivo administration of the nucleic acid sequence is configured to produce one or more peptides that are displayed by an HLA class I molecule. In some embodiments, the one or more peptides is a modified or unmodified fragment of a mutated KRAS protein. In some embodiments, the mutated KRAS protein is selected from the group consisting of KRAS G12D, KRAS G12V, and KRAS G12R. In some embodiments, the nucleic acid sequence is administered in an effective amount to a subject to prevent cancer. In some embodiments, the nucleic acid sequence is administered in an effective amount to a subject to treat cancer.

In another aspect, the invention provides for an immunogenic peptide composition comprising two or more peptides selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In some embodiments, a peptide in the immunogenic peptide composition is displayed by an HLA class I molecule. In some embodiments, a peptide in the immunogenic peptide composition is a modified or unmodified fragment of a mutated KRAS protein. In some embodiments, the mutated KRAS protein is selected from the group consisting of KRAS G12D, KRAS G12V, and KRAS G12R. In some embodiments, the immunogenic peptide composition is administered in an effective amount to a subject to prevent cancer. In some embodiments, the immunogenic peptide composition is administered in an effective amount to a subject to treat cancer. In some embodiments, the immunogenic peptide composition comprises at least three peptides selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, and SEQ ID NO: 41.

In another aspect, the invention provides for a nucleic acid sequence encoding one or more amino acid sequences selected from the group consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

In some embodiments, the nucleic acid sequence is an immunogenic composition. In some embodiments, the nucleic acid sequence is administered in a construct for expression in vivo. In some embodiments, the in vivo administration of the nucleic acid sequence is configured to produce one or more peptides that are displayed by an HLA class II molecule. In some embodiments, the one or more peptides is a modified fragment of a mutated KRAS protein. In some embodiments, the mutated KRAS protein is selected from the group consisting of KRAS G12D, KRAS G12V, KRAS G12R, KRAS G12C, and KRAS G13D. In some embodiments, the nucleic acid sequence is administered in an effective amount to a subject to prevent cancer. In some embodiments, the nucleic acid sequence is administered in an effective amount to a subject to treat cancer.

In another aspect, the invention provides for an immunogenic peptide composition comprising one or more peptides selected from the group consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

In some embodiments, a peptide in the immunogenic peptide composition is displayed by an HLA class II molecule. In some embodiments, a peptide in the immunogenic peptide composition is a modified or unmodified fragment of a mutated KRAS protein. In some embodiments, the mutated KRAS protein is selected from the group consisting of KRAS G12D, KRAS G12V, KRAS G12R, KRAS G12C, and KRAS G13D. In some embodiments, the immunogenic peptide composition is administered in an effective amount to a subject to prevent cancer. In some embodiments, the immunogenic peptide composition is administered in an effective amount to a subject to treat cancer. In some embodiments, the immunogenic peptide composition comprises at least two peptides selected from the group consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64, and SEQ ID NO: 65.

### Compositions

In some embodiments, the foreign peptides (e.g., peptide vaccine) are administered in a pharmaceutical composition comprising the peptides and a pharmaceutically acceptable carrier. In some embodiments, the pharmaceutical composition is in the form of a spray, aerosol, gel, solution, emulsion, or suspension.

The composition is preferably administered to a subject with a pharmaceutically acceptable carrier. Typically, in some embodiments, an appropriate amount of a pharmaceutically acceptable salt is used in the formulation, which in some embodiments can render the formulation isotonic.

In certain embodiments, the foreign peptides are provided as an immunogenic composition comprising any one of the foreign peptides described herein and a pharmaceutically acceptable carrier. In certain embodiments, the immunogenic composition further comprises an adjuvant. In certain embodiments, the foreign peptides are conjugated with other molecules to increase their effectiveness as is known by those practiced in the art. For example, peptides can be coupled to antibodies that recognize cell surface proteins on antigen presenting cells to enhance vaccine effectiveness. One such method for increasing the effectiveness of peptide delivery is described in Woodham, et al. (2018).

In some embodiments, the pharmaceutically acceptable carrier is selected from the group consisting of saline, Ringer's solution, dextrose solution, and a combination thereof. Other suitable pharmaceutically acceptable carriers known in the art are contemplated. Suitable carriers and their formulations are described in Remington's Pharmaceutical Sciences, 2005, Mack Publishing Co. The pH of the solution is preferably from about 5 to about 8, and more preferably from about 7 to about 7.5. The formulation may also comprise a lyophilized powder. Further carriers include sustained release preparations such as semipermeable matrices of solid hydrophobic polymers, which matrices are in the form of shaped articles, e.g., films, liposomes or microparticles. It will be apparent to those persons skilled in the art that certain carriers may be more preferable depending upon, for instance, the route of administration and concentration of peptides being administered.

The phrase pharmaceutically acceptable carrier as used herein means a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier is acceptable in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as butylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. The term carrier denotes an organic or inorganic ingredient, natural or synthetic, with which the active ingredient is combined to facilitate the application. The components of the pharmaceutical compositions also are capable of being comingled with the compounds of the present invention, and with each other, in a manner such that there is no interaction which would substantially impair the desired pharmaceutical efficiency. The composition may also include additional agents such as an isotonicity agent, a preservative, a surfactant, and, a divalent cation, preferably, zinc.

The composition can also include an excipient, or an agent for stabilization of a foreign peptide composition, such as a buffer, a reducing agent, a bulk protein, amino acids (such as e.g., glycine or praline) or a carbohydrate. Bulk proteins useful in formulating foreign peptide compositions include albumin. Typical carbohydrates useful in formulating foreign peptides include but are not limited to sucrose, mannitol, lactose, trehalose, or glucose.

Surfactants may also be used to prevent soluble and insoluble aggregation and/or precipitation of foreign peptides or proteins included in the composition. Suitable surfactants include but are not limited to sorbitan trioleate, soya lecithin, and oleic acid. In certain cases, solution aerosols are preferred using solvents such as ethanol. Thus, formulations including foreign peptides can also include a surfactant that can reduce or prevent surface-induced aggregation of foreign peptides by atomization of the solution in forming an aerosol. Various conventional surfactants can be employed, such as polyoxyethylene fatty acid esters and alcohols, and polyoxyethylene sorbitol fatty acid esters. Amounts will generally range between 0.001% and 4% by weight of the formulation. In some embodiments, surfactants used with the present disclosure are polyoxyethylene sorbitan mono-oleate, polysorbate 80, polysorbate 20. Additional agents known in the art can also be included in the composition.

In some embodiments, the pharmaceutical compositions and dosage forms further comprise one or more compounds that reduce the rate by which an active ingredient will decay, or the composition will change in character. So called stabilizers or preservatives may include, but are not limited to, amino acids, antioxidants, pH buffers, or salt buffers. Nonlimiting examples of antioxidants include butylated hydroxy anisole (BHA), ascorbic acid and derivatives thereof, tocopherol and derivatives thereof, butylated hydroxy anisole and cysteine. Nonlimiting examples of preservatives include parabens, such as methyl or propyl p-hydroxybenzoate and benzalkonium chloride. Additional nonlimiting examples of amino acids include glycine or proline.

The present invention also teaches the stabilization (preventing or minimizing thermally or mechanically induced soluble or insoluble aggregation and/or precipitation of an inhibitor protein) of liquid solutions containing foreign peptides at neutral pH or less than neutral pH by the use of amino acids including proline or glycine, with or without divalent cations resulting in clear or nearly clear solutions that are stable at room temperature or preferred for pharmaceutical administration.

In one embodiment, the composition is a pharmaceutical composition of single unit or multiple unit dosage forms. Pharmaceutical compositions of single unit or multiple unit dosage forms of the invention comprise a prophylactically or therapeutically effective amount of one or more compositions (e.g., a compound of the invention, or other prophylactic or therapeutic agent), typically, one or more vehicles, carriers, or excipients, stabilizing agents, and/or preservatives. Preferably, the vehicles, carriers, excipients, stabilizing agents and preservatives are pharmaceutically acceptable.

In some embodiments, the pharmaceutical compositions and dosage forms comprise anhydrous pharmaceutical compositions and dosage forms. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprise a primary or secondary amine are preferably anhydrous if substantial contact with moisture and/or humidity during manufacturing, packaging, and/or storage is expected. An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

Suitable vehicles are well known to those skilled in the art of pharmacy, and non-limiting examples of suitable vehicles include glucose, sucrose, starch, lactose, gelatin, rice, silica gel, glycerol, talc, sodium chloride, dried skim milk, propylene glycol, water, sodium stearate, ethanol, and similar substances well known in the art. Saline solutions and aqueous dextrose and glycerol solutions can also be employed as liquid vehicles. Whether a particular vehicle is suitable for incorporation into a pharmaceutical composition or dosage form depends on a variety of factors well known in the art including, but not limited to, the way in which the dosage form will be administered to a patient and the specific active ingredients in the dosage form. Pharmaceutical vehicles can be sterile liquids, such as water and oils, including those of petroleum, animal, vegetable or synthetic origin, such as peanut oil, soybean oil, mineral oil, sesame oil and the like.

The invention also provides that a pharmaceutical composition can be packaged in a hermetically sealed container such as an ampoule or sachette indicating the quantity. In one embodiment, the pharmaceutical composition can be supplied as a dry sterilized lyophilized powder in a delivery device suitable for administration to the lower airways of a patient. The pharmaceutical compositions can, if desired, be presented in a pack or dispenser device that can contain one or more unit dosage forms containing the active ingredient. The pack can for example comprise metal or plastic foil, such as a blister pack. The pack or dispenser device can be accompanied by instructions for administration.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for administration may be in the form of powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouthwashes and the like, each containing a predetermined amount of a compound of the present invention (e.g., peptides) as an active ingredient.

A liquid composition herein can be used as such with a delivery device, or they can be used for the preparation of pharmaceutically acceptable formulations comprising foreign peptides that are prepared for example by the method of spray drying. The methods of spray freeze-drying foreign peptides/proteins for pharmaceutical administration disclosed in Maa et al., Curr. Pharm. Biotechnol., 2001, 1, 283-302, are incorporated herein. In another embodiment, the liquid solutions herein are freeze spray dried and the spray-dried product is collected as a dispersible foreign peptide-containing powder that is therapeutically effective when administered to an individual.

The compounds and pharmaceutical compositions of the present invention can be employed in combination therapies, that is, the compounds and pharmaceutical compositions can be administered concurrently with, prior to, or subsequent to, one or more other desired therapeutics or medical procedures (e.g., foreign peptide vaccine can be used in combination therapy with another treatment such as chemotherapy, radiation, pharmaceutical agents, and/or another treatment). The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, the compound of the present invention may be administered concurrently with another therapeutic or prophylactic).

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The current invention provides for dosage forms comprising foreign peptides suitable for treating cancer or other diseases. The dosage forms can be formulated, e.g., as sprays, aerosols, nanoparticles, liposomes, or other forms known to one of skill in the art. See, e.g., Remington's Pharmaceutical Sciences; Remington: The Science and Practice of Pharmacy supra; Pharmaceutical Dosage Forms and Drug Delivery Systems by Howard C., Ansel et al., Lippincott Williams & Wilkins; 7th edition (Oct. 1, 1999).

Generally, a dosage form used in the acute treatment of a disease may contain larger amounts of one or more of the active ingredients it comprises than a dosage form used in the chronic treatment of the same disease. In addition, the prophylactically and therapeutically effective dosage form may vary among different conditions. For example, a therapeutically effective dosage form may contain peptides that has an appropriate immunogenic action when intending to treat cancer or other disease. On the other hand, a different effective dosage may contain foreign peptides that has an appropriate immunogenic action when intending to use the peptides of the invention as a prophylactic (e.g., vaccine) against cancer or another disease/condition. These and other ways in which specific dosage forms encompassed by this invention will vary from one another and will be readily apparent to those skilled in the art. See, e.g., Remington's Pharmaceutical Sciences, 2005, Mack Publishing Co.; Remington: The Science and Practice of Pharmacy by Gennaro, Lippincott Williams & Wilkins; 20th edition (2003); Pharmaceutical Dosage Forms and Drug Delivery Systems by Howard C. Ansel et al., Lippincott Williams & Wilkins; 7th edition (Oct. 1, 1999); and Encyclopedia of Pharmaceutical Technology, edited by Swarbrick, J. & J. C. Boylan, Marcel Dekker, Inc., New York, 1988, which are incorporated herein by reference in their entirety.

The pH of a pharmaceutical composition or dosage form may also be adjusted to improve delivery and/or stability of one or more active ingredients. Similarly, the polarity of a solvent carrier, its ionic strength, or tonicity can be adjusted to improve delivery. Compounds such as stearates can also be added to pharmaceutical compositions or dosage forms to alter advantageously the hydrophilicity or lipophilicity of one or more active ingredients to improve delivery. In this regard, stearates can also serve as a lipid vehicle for the formulation, as an emulsifying agent or surfactant, and as a delivery enhancing or penetration-enhancing agent. Different salts, hydrates, or solvates of the active ingredients can be used to adjust further the properties of the resulting composition.

Compositions can be formulated with appropriate carriers and adjuvants using techniques to yield compositions suitable for immunization. The compositions can include an adjuvant, such as, for example but not limited to, alum, poly IC, MF-59, squalene-based adjuvants, or liposomal based adjuvants suitable for immunization.

In some embodiments, the compositions and methods comprise any suitable agent or immune modulation which could modulate mechanisms of host immune tolerance and release of the induced antibodies. In certain embodiments, an immunomodulatory agent is administered in at time and in an amount sufficient for transient modulation of the subject's immune response so as to induce an immune response which comprises antibodies against for example tumor neoantigens (i.e., tumor-specific antigens (TSA)).

### Expression Systems

In certain aspects, the invention provides culturing a cell line that expresses any one of the foreign peptides of the invention in a culture medium comprising any of the foreign peptides described herein.

Various expression systems for producing recombinant proteins/peptides are known in the art, and include, prokaryotic (e.g., bacteria), plant, insect, yeast, and mammalian expression systems. Suitable cell lines, can be transformed, transduced, or transfected with nucleic acids containing coding sequences for the foreign peptides of the invention in order to produce the molecule of interest. Expression vectors containing such a nucleic acid sequence, which can be linked to at least one regulatory sequence in a manner that allows expression of the nucleotide sequence in a host cell, can be introduced via methods known in the art. Practitioners in the art understand that designing an expression vector can depend on factors, such as the choice of host cell to be transfected and/or the type and/or amount of desired protein to be expressed. Enhancer regions, which are those sequences found upstream or downstream of the promoter region in non-coding DNA regions, are also known in the art to be important in optimizing expression. If needed, origins of replication from viral sources can be employed, such as if a prokaryotic host is utilized for introduction of plasmid DNA. However, in eukaryotic organisms, chromosome integration is a common mechanism for DNA replication. For stable transfection of mammalian cells, a small fraction of cells can integrate introduced DNA into their genomes. The expression vector and transfection method utilized can be factors that contribute to a successful integration event. For stable amplification and expression of a desired protein, a vector containing DNA encoding a protein of interest is stably integrated into the genome of eukaryotic cells (for example mammalian cells), resulting in the stable expression of transfected genes. A gene that encodes a selectable marker (for example, resistance to antibiotics or drugs) can be introduced into host cells along with the gene of interest in order to identify and select clones that stably express a gene encoding a protein of interest. Cells containing the gene of interest can be identified by drug selection wherein cells that have incorporated the selectable marker gene will survive in the presence of the drug. Cells that have not incorporated the gene for the selectable marker die. Surviving cells can then be screened for the production of the desired protein molecule.

A host cell strain, which modulates the expression of the inserted sequences, or modifies and processes the nucleic acid in a specific fashion desired also may be chosen. Such modifications (for example, glycosylation and other post-translational modifications) and processing (for example, cleavage) of peptide/protein products may be important for the function of the peptide/protein. Different host cell strains have characteristic and specific mechanisms for the post-translational processing and modification of proteins and gene products. As such, appropriate host systems or cell lines can be chosen to ensure the correct modification and processing of the foreign protein expressed. Thus, eukaryotic host cells possessing the cellular machinery for proper processing of the primary transcript, glycosylation, and phosphorylation of the gene product may be used.

Various culturing parameters can be used with respect to the host cell being cultured. Appropriate culture conditions for mammalian cells are well known in the art (Cleveland W L, et al., J Immunol Methods, 1983, 56(2): 221-234) or can be determined by the skilled artisan (see, for example, Animal Cell Culture: A Practical Approach 2nd Ed., Rickwood, D. and Hames, B. D., eds. (Oxford University Press: New York, 1992)). Cell culturing conditions can vary according to the type of host cell selected. Commercially available medium can be utilized.

Foreign peptides of the invention can be purified from any human or non-human cell which expresses the polypeptide, including those which have been transfected with expression constructs that express foreign peptides of the invention. For protein recovery, isolation and/or purification, the cell culture medium or cell lysate is centrifuged to remove particulate cells and cell debris. The desired polypeptide molecule is isolated or purified away from contaminating soluble proteins and polypeptides by suitable purification techniques. Non-limiting purification methods for proteins include: size exclusion chromatography; affinity chromatography; ion exchange chromatography; ethanol precipitation; reverse phase HPLC; chromatography on a resin, such as silica, or cation exchange resin, e.g., DEAE; chromatofocusing; SDS-PAGE; ammonium sulfate precipitation; gel filtration using, e.g., Sephadex G-75, Sepharose; protein A sepharose chromatography for removal of immunoglobulin contaminants; and the like. Other additives, such as protease inhibitors (e.g., PMSF or proteinase K) can be used to inhibit proteolytic degradation during purification. Purification procedures that can select for carbohydrates can also be used, e.g., ion-exchange soft gel chromatography, or HPLC using cation- or anionexchange resins, in which the more acidic fraction(s) is/are collected.

### Methods of Treatment

In one embodiment, the subject matter disclosed herein relates to a preventive medical treatment started after following diagnosis of cancer in order to prevent the disease from worsening or curing the disease. In one embodiment, the subject matter disclosed herein relates to prophylaxis of subjects who are believed to be at risk for cancer or have previously been diagnosed with cancer (or another disease). In one embodiment, said subjects can be administered the peptide vaccine described herein or pharmaceutical compositions thereof. The invention contemplates using any of the foreign peptides produced by the systems and methods described herein. In one embodiment, the foreign peptide vaccines described herein can be administered subcutaneously via syringe or any other suitable method know in the art.

The compound(s) or combination of compounds disclosed herein, or pharmaceutical compositions may be administered to a cell, mammal, or human by any suitable means. Non-limiting examples of methods of administration include, among others, (a) administration though oral pathways, which includes administration in capsule, tablet, granule, spray, syrup, or other such forms; (b) administration through non-oral pathways such as intraocular, intranasal, intraauricular, rectal, vaginal, intraurethral, transmucosal, buccal, or transdermal, which includes administration as an aqueous suspension, an oily preparation or the like or as a drip, spray, suppository, salve, ointment or the like; (c) administration via injection, including subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; (e) administration topically; as deemed appropriate by those of skill in the art for bringing the compound or combination of compounds disclosed herein into contact with living tissue; (f) administration via inhalation, including through aerosolized, nebulized, and powdered formulations; and (g) administration through implantation.

As will be readily apparent to one skilled in the art, the effective in vivo dose to be administered and the particular mode of administration will vary depending upon the age, weight and species treated, and the specific use for which the compound or combination of compounds disclosed herein are employed. The determination of effective dose levels, that is the dose levels necessary to achieve the desired result, can be accomplished by one skilled in the art using routine pharmacological methods. Typically, human clinical applications of products are commenced at lower dose levels, with dose level being increased until the desired effect is achieved. Alternatively, acceptable in vitro studies can be used to establish useful doses and routes of administration of the compositions identified by the present methods using established pharmacological methods. Effective animal doses from in vivo studies can be converted to appropriate human doses using conversion methods known in the art (e.g., see Nair A B, Jacob S. A simple practice guide for dose conversion between animals and human. Journal of basic and clinical pharmacy. 2016 March; 7(2):27.)

### Methods of Prevention

In some embodiments, the foreign peptides prepared using methods of the invention can be used as a vaccine to promote an immune response against cancer (e.g., against tumor neoantigens). In some embodiments, the invention provides compositions and methods for induction of immune response, for example induction of antibodies to tumor neoantigens. In some embodiments, the antibodies are broadly neutralizing antibodies. In some embodiments, the foreign peptides prepared using methods of the invention can be used as a vaccine to promote an immune response against a pathogen.

The compositions, systems, and methods disclosed herein are not to be limited in scope to the specific embodiments described herein. Indeed, various modifications of the compositions, systems, and methods in addition to those described will become apparent to those of skill in the art from the foregoing description.

What is claimed:

1. A composition comprising one or more polynucleotides, wherein the one or more polynucleotides comprise one or more polynucleotide sequences encoding one or more amino acid sequences selected from the group consisting of SEQ ID NO: 1, SEQ ID NO: 2, SEQ ID NO: 21, SEQ ID NO: 23, SEQ ID NO: 86, SEQ ID NO: 88, SEQ ID NO: 125, SEQ ID NO: 154, SEQ ID NO: 194, SEQ ID NO: 200, SEQ ID NO: 209, and SEQ ID NO: 366, wherein each amino acid sequence of the one or more acid sequences encode a fragment of a KRAS protein, wherein the fragment of the KRAS protein comprises a G12D mutation and a heteroclitic modification.

2. The composition of claim 1, wherein the one or more polynucleotides are contained in a construct for in vivo expression in a subject of one or more peptides encoded by the one or more amino acid sequences.

3. The composition of claim 2, wherein an administration of the one or more polynucleotides causes the one or more peptides encoded by the one or more amino acid sequences to be displayed by a HLA class I molecule in the subject.

4. The composition of claim 3, wherein the one or more peptides are displayed by the HLA class I molecule with a peptide-HLA binding affinity value of less than about 500 nM.

5. The composition of claim 1, wherein the one or more polynucleotide sequences encode at least two amino acid sequences selected from the group consisting of SEQ ID NO: 1, SEQ ID NO: 2, SEQ ID NO: 21, SEQ ID NO: 23, SEQ ID NO: 86, SEQ ID NO: 88, SEQ ID NO: 125, SEQ ID NO: 154, SEQ ID NO: 194, SEQ ID NO: 200, SEQ ID NO: 209, and SEQ ID NO: 366.

6. The composition of claim 5, wherein the one or more polynucleotides are contained in a construct for in vivo expression in a subject of at least two peptides encoded by the at least two amino acid sequences.

7. The composition of claim 6, wherein an administration of the one or more polynucleotides causes the at least two peptides encoded by the at least two amino acid sequences to be displayed by a HLA class I molecule in the subject.

8. The composition of claim 7, wherein the administration of the one or more polynucleotides causes:
a first peptide of the at least two peptides to be displayed by a first plurality of HLA class I alleles; and
a second peptide of the at least two peptides to be displayed by a second plurality of HLA class I alleles,
wherein the first plurality of HLA class I alleles and the second plurality of HLA class I alleles differ by at least one HLA class I allele.

9. The composition of claim 2, wherein the composition is an immunogenic composition and is configured to prevent or treat cancer in the subject.

10. The composition of claim 1, wherein the one or more polynucleotides encode each of SEQ ID NO: 1, SEQ ID NO: 2, SEQ ID NO: 21, SEQ ID NO: 23, SEQ ID NO: 86, SEQ ID NO: 88, SEQ ID NO: 125, SEQ ID NO: 154, SEQ ID NO: 194, SEQ ID NO: 200, SEQ ID NO: 209, and SEQ ID NO: 366.

11. A composition comprising one or more polynucleotides, wherein the one or more polynucleotides comprise one or more polynucleotide sequences encoding one or more amino acid sequences selected from the group consisting of SEQ ID NO: 3, SEQ ID NO: 4, SEQ ID NO: 6, SEQ ID NO: 15, SEQ ID NO: 16, SEQ ID NO: 69, SEQ ID NO: 72, SEQ ID NO: 118, SEQ ID NO: 122, SEQ ID NO: 131, SEQ ID NO: 138, SEQ ID NO: 152, SEQ ID NO: 189, SEQ ID NO: 233, SEQ ID NO: 353, SEQ ID NO: 514, and SEQ ID NO: 591, wherein each amino acid sequence of the one or more amino acid sequences encodes a fragment of a KRAS protein, wherein the fragment of the KRAS protein comprises a G12V mutation and a heteroclitic modification.

12. The composition of claim 11, wherein the one or more polynucleotides are contained in a construct for in vivo expression in a subject of one or more peptides encoded by the one or more amino acid sequences.

13. The composition of claim 12, wherein an administration of the one or more polynucleotides causes the one or more peptides encoded by the one or more amino acid sequences to be displayed by a HLA class I molecule in the subject.

14. The composition of claim 13, wherein the one or more peptides are displayed by the HLA class I molecule with a peptide-HLA binding affinity value of less than about 500 nM.

15. The composition of claim 11, wherein the one or more polynucleotide sequences encode at least two amino acid sequence selected from the group consisting of SEQ ID NO: 3, SEQ ID NO: 4, SEQ ID NO: 6, SEQ ID NO: 15, SEQ ID NO: 16, SEQ ID NO: 69, SEQ ID NO: 72, SEQ ID NO: 118, SEQ ID NO: 122, SEQ ID NO: 131, SEQ ID NO: 138, SEQ ID NO: 152, SEQ ID NO: 189, SEQ ID NO: 233, SEQ ID NO: 353, SEQ ID NO: 514, and SEQ ID NO: 591.

16. The composition of claim 15, wherein the one or more polynucleotides are contained in a construct for in vivo expression in a subject of at least two peptides encoded by the at least two amino acid sequence.

17. The composition of claim 16, wherein an administration of the one or more polynucleotides causes the at least two peptides encoded by the at least two amino acid sequences to be displayed by a HLA class I molecule in the subject.

18. The composition of claim 17, wherein the administration of the one or more polynucleotides causes:
a first peptide of the at least two peptides to be displayed by a first plurality of HLA class I alleles; and
a second peptide of the at least two peptides to be displayed by a second plurality of HLA class I alleles,
wherein the first plurality of HLA class I alleles and the second plurality of HLA class I alleles differ by at least one HLA class I allele.

19. The composition of claim 12, wherein the composition is an immunogenic composition and is configured to prevent or treat cancer in the subject.

20. The composition of claim 11, wherein the one or more polynucleotides encode each of SEQ ID NO: 3, SEQ ID NO: 4, SEQ ID NO: 6, SEQ ID NO: 15, SEQ ID NO: 16, SEQ ID NO: 69, SEQ ID NO: 72, SEQ ID NO: 118, SEQ ID NO: 122, SEQ ID NO: 131, SEQ ID NO: 138, SEQ ID NO: 152, SEQ ID NO: 189, SEQ ID NO: 233, SEQ ID NO: 353, SEQ ID NO: 514, and SEQ ID NO: 591.

21. A composition comprising one or more polynucleotides, wherein the one or more polynucleotides comprise one or more polynucleotide sequences encoding one or more amino acid sequences selected from the group consisting of SEQ ID NO: 5, SEQ ID NO: 8, SEQ ID NO: 9, SEQ ID NO: 10, SEQ ID NO: 17, SEQ ID NO: 18, SEQ ID NO: 19, SEQ ID NO: 20, SEQ ID NO: 25, SEQ ID NO: 113, SEQ ID NO: 181, SEQ ID NO: 195, SEQ ID NO: 212, and SEQ ID NO: 324, wherein each amino acid sequence of the one or more amino acid sequences encodes a fragment of a KRAS protein, wherein the fragment of the KRAS protein comprises a G12R mutation and a heteroclitic modification.

22. The composition of claim 21, wherein the one or more polynucleotides are contained in a construct for in vivo expression in a subject of one or more peptides encoded by the one or more amino acid sequences.

23. The composition of claim 22, wherein an administration of the one or more polynucleotides causes the one or more peptides encoded by the one or more amino acid sequences to be displayed by a HLA class I molecule in the subject.

24. The composition of claim 23, wherein the one or more peptides are displayed by the HLA class I molecule with a peptide-HLA binding affinity value of less than about 500 nM.

25. The composition of claim 21, wherein the one or more polynucleotide sequences encode at least two amino acid sequence selected from the group consisting of SEQ ID NO: 5, SEQ ID NO: 8, SEQ ID NO: 9, SEQ ID NO: 10, SEQ ID NO: 17, SEQ ID NO: 18, SEQ ID NO: 19, SEQ ID NO: 20, SEQ ID NO: 25, SEQ ID NO: 113, SEQ ID NO: 181, SEQ ID NO: 195, SEQ ID NO: 212, and SEQ ID NO: 324.

26. The composition of claim 25, wherein the one or more polynucleotides are contained in a construct for in vivo expression in a subject of at least two peptides encoded by the at least two amino acid sequence.

27. The composition of claim 26, wherein an administration of the one or more polynucleotides causes the at least two peptides encoded by the at least two amino acid sequences to be displayed by a HLA class I molecule in the subject.

28. The composition of claim 27, wherein the administration of the one or more polynucleotides causes:
a first peptide of the at least two peptides to be displayed by a first plurality of HLA class I alleles; and
a second peptide of the at least two peptides to be displayed by a second plurality of HLA class I alleles,
wherein the first plurality of HLA class I alleles and the second plurality of HLA class I alleles differ by at least one HLA class I allele.

29. The composition of claim 22, wherein the composition is an immunogenic composition and is configured to prevent or treat cancer in the subject.

30. The composition of claim 21, wherein the one or more polynucleotides encode each of SEQ ID NO: 5, SEQ ID NO: 8, SEQ ID NO: 9, SEQ ID NO: 10, SEQ ID NO: 17, SEQ ID NO: 18, SEQ ID NO: 19, SEQ ID NO: 20, SEQ ID NO: 25, SEQ ID NO: 113, SEQ ID NO: 181, SEQ ID NO: 195, SEQ ID NO: 212, and SEQ ID NO: 324.

31. A composition comprising one or more polynucleotides, wherein the one or more polynucleotides comprise one or more polynucleotide sequences encoding one or more amino acid sequences selected from the group consisting of SEQ ID NO: 26, SEQ ID NO: 27, SEQ ID NO: 28, SEQ ID NO: 30, SEQ ID NO: 36, SEQ ID NO: 37, SEQ ID NO: 70, SEQ ID NO: 71, SEQ ID NO: 73, SEQ ID NO: 74, SEQ ID NO: 78, SEQ ID NO: 176, and SEQ ID NO: 187, wherein each amino acid sequence of the one or more amino acid sequences encodes a fragment of a KRAS protein, wherein the fragment of the KRAS protein comprises a G12C mutation and a heteroclitic modification.

32. The composition of claim 31, wherein the one or more polynucleotides are contained in a construct for in vivo expression in a subject of one or more one peptides encoded by the one or more amino acid sequences.

33. The composition of claim 32, wherein an administration of the one or more polynucleotides causes the one or more peptides encoded by the one or more amino acid sequences to be displayed by a HLA class I molecule in the subject.

34. The composition of claim 33, wherein the one or more peptides are displayed by the HLA class I molecule with a peptide-HLA binding affinity value of less than about 500 nM.

35. The composition of claim 31, wherein the one or more polynucleotide sequences encode at least two amino acid sequence selected from the group consisting of SEQ ID NO: 26, SEQ ID NO: 27, SEQ ID NO: 28, SEQ ID NO: 30, SEQ ID NO: 36, SEQ ID NO: 37, SEQ ID NO: 70, SEQ ID NO: 71, SEQ ID NO: 73, SEQ ID NO: 74, SEQ ID NO: 78, SEQ ID NO: 176, and SEQ ID NO: 187.

36. The composition of claim 35, wherein the one or more polynucleotides are contained in a construct for in vivo expression in a subject of at least two peptides encoded by the at least two amino acid sequence.

37. The composition of claim 36, wherein an administration of the one or more polynucleotides causes the at least two peptides encoded by the at least two amino acid sequences to be displayed by a HLA class I molecule in the subject.

38. The composition of claim 37, wherein the administration of the one or more polynucleotides causes:
a first peptide of the at least two peptides to be displayed by a first plurality of HLA class I alleles; and
a second peptide of the at least two peptides to be displayed by a second plurality of HLA class I alleles,
wherein the first plurality of HLA class I alleles and the second plurality of HLA class I alleles differ by at least one HLA class I allele.

39. The composition of claim 32, wherein the composition is an immunogenic composition and is configured to prevent or treat cancer in the subject.

40. The composition of claim 31, wherein the one or more polynucleotides encode each of SEQ ID NO: 26, SEQ ID NO: 27, SEQ ID NO: 28, SEQ ID NO: 30, SEQ ID NO: 36, SEQ ID NO: 37, SEQ ID NO: 70, SEQ ID NO: 71, SEQ ID NO: 73, SEQ ID NO: 74, SEQ ID NO: 78, SEQ ID NO: 176, and SEQ ID NO: 187.


SEQUENCE LISTING (1523 sequences)

SEQ ID NO: 1 - protein, 9 aa, synthetic construct
  source 1..9: note = Seed: GADGVGKSA (KRAS G12D)
GADGVGKSM

SEQ ID NO: 2 - protein, 9 aa, synthetic construct
  source 1..9: note = Seed: LVVVGADGV (KRAS G12D)
LMVVGADGV

SEQ ID NO: 3 - protein, 9 aa, synthetic construct
  source 1..9: note = Seed: GAVGVGKSA (KRAS G12V)
GAVGVGKSL

SEQ ID NO: 4 - protein, 9 aa, synthetic construct
  source 1..9: note = Seed: LVVVGAVGV (KRAS G12V)
LMVVGAVGV

SEQ ID NO: 5 - protein, 9 aa, synthetic construct
  source 1..9: note = Seed: VVGARGVGK (KRAS G12R)
VTGARGVGK

SEQ ID NO: 6 - protein, 9 aa, synthetic construct
  source 1..9: note = Seed: VVGAVGVGK (KRAS G12V)
VMGAVGVGK

SEQ ID NO: 7 - protein, 9 aa, Homo sapiens
  source 1..9: note = Native (KRAS G12V)
VVGAVGVGK

SEQ ID NO: 8 - protein, 9 aa, synthetic construct
  source 1..9: note = Seed: GARGVGKSA (KRAS G12R)
GARGVGKSY

SEQ ID NO: 9 - protein, 9 aa, synthetic construct
  source 1..9: note = Seed: GARGVGKSA (KRAS G12R)
GPRGVGKSA

SEQ ID NO: 10 - protein, 9 aa, synthetic construct
  source 1..9: note = Seed: LVVVGARGV (KRAS G12R)
LMVVGARGV

SEQ ID NO: 11 - protein, 9 aa, synthetic construct
  source 1..9: note = Seed: GADGVGKSA (KRAS G12D)
GADGVGKSL

SEQ ID NO: 12 - protein, 9 aa, synthetic construct
  source 1..9: note = Seed: GADGVGKSA (KRAS G12D)
GADGVGKSY

SEQ ID NO: 13 - protein, 9 aa, synthetic construct
  source 1..9: note = Seed: GADGVGKSA (KRAS G12D)
GYDGVGKSM

SEQ ID NO: 14 - protein, 9 aa, synthetic construct
  source 1..9: note = Seed: GAVGVGKSA (KRAS G12V)
GPVGVGKSV

SEQ ID NO: 15 - protein, 9 aa, synthetic construct
  source 1..9: note = Seed: LVVVGAVGV (KRAS G12V)
LTVVGAVGV

SEQ ID NO: 16 - protein, 9 aa, synthetic construct
  source 1..9: note = Seed: VVGAVGVGK (KRAS G12V)
VVGAVGVGR

SEQ ID NO: 17 - protein, 9 aa, synthetic construct
  source 1..9: note = Seed: GARGVGKSA (KRAS G12R)
GARGVGKSM

SEQ ID NO: 18 - protein, 9 aa, synthetic construct
  source 1..9: note = Seed: GARGVGKSA (KRAS G12R)
GPRGVGKSV

SEQ ID NO: 19 - protein, 9 aa, synthetic construct
  source 1..9: note = Seed: LVVVGARGV (KRAS G12R)
LLVVGARGV

SEQ ID NO: 20 - protein, 9 aa, synthetic construct
  source 1..9: note = Seed: VVGARGVGK (KRAS G12R)
VAGARGVGM

SEQ ID NO: 21 - protein, 9 aa, synthetic construct
  source 1..9: note = Seed: LVVVGADGV (KRAS G12D)
LTVVGADGV

SEQ ID NO: 22 - protein, 9 aa, synthetic construct
  source 1..9: note = Seed: LVVVGADGV (KRAS G12D)
LLVVGADGV

SEQ ID NO: 23 - protein, 9 aa, synthetic construct
  source 1..9: note = Seed: LVVVGADGV (KRAS G12D)
LMVVGADGL

SEQ ID NO: 24 - protein, 9 aa, synthetic construct
  source 1..9: note = Seed: VVGAVGVGK (KRAS G12V)
VMGAVGVGR

SEQ ID NO: 25 - protein, 9 aa, synthetic construct
  source 1..9: note = Seed: VVGARGVGK (KRAS G12R)
VMGARGVGK

SEQ ID NO: 26 - protein, 9 aa, synthetic construct
  source 1..9: note = Seed: GACGVGKSA (KRAS G12C)
GACGVGKSL

SEQ ID NO: 27 - protein, 9 aa, synthetic construct
  source 1..9: note = Seed: LVVVGACGV (KRAS G12C)
LMVVGACGV

SEQ ID NO: 28 - protein, 9 aa, synthetic construct
  source 1..9: note = Seed: LVVVGACGV (KRAS G12C)
LTVVGACGV

SEQ ID NO: 29 - protein, 9 aa, synthetic construct
  source 1..9: note = Seed: VVGACGVGK (KRAS G12C)
VTGACGVGK

SEQ ID NO: 30 - protein, 9 aa, synthetic construct
  source 1..9: note = Seed: VVGACGVGK (KRAS G12C)
VVGACGVGR

SEQ ID NO: 31 - protein, 9 aa, synthetic construct
  source 1..9: note = Seed: AGDVGKSAL (KRAS G13D)
AADVGKSAM

SEQ ID NO: 32 - protein, 9 aa, synthetic construct
  source 1..9: note = Seed: AGDVGKSAL (KRAS G13D)
AEDVGKSAM

SEQ ID NO: 33 - protein, 9 aa, synthetic construct
  source 1..9: note = Seed: AGDVGKSAL (KRAS G13D)
AYDVGKSAM

SEQ ID NO: 34 - protein, 9 aa, synthetic construct
  source 1..9: note = Seed: DVGKSALTI (KRAS G13D)
DAGKSALTV

SEQ ID NO: 35 - protein, 9 aa, synthetic construct
  source 1..9: note = Seed: GAGDVGKSA (KRAS G13D)
GAGDVGKSM

SEQ ID NO: 36 - protein, 9 aa, synthetic construct
  source 1..9: note = Seed: LVVVGACGV (KRAS G12C)
LQVVGACGV

SEQ ID NO: 37 - protein, 9 aa, synthetic construct
  source 1..9: note = Seed: VVGACGVGK (KRAS G12C)
VMGACGVGK

SEQ ID NO: 38 - protein, 9 aa, synthetic construct
  source 1..9: note = Seed: VVGACGVGK (KRAS G12C)
VMGACGVGR

SEQ ID NO: 39 - protein, 9 aa, synthetic construct
  source 1..9: note = Seed: AGDVGKSAL (KRAS G13D)
AADVGKSAL

SEQ ID NO: 40 - protein, 9 aa, synthetic construct
  source 1..9: note = Seed: AGDVGKSAL (KRAS G13D)
ASDVGKSAL

SEQ ID NO: 41 - protein, 9 aa, synthetic construct
  source 1..9: note = Seed: AGDVGKSAL (KRAS G13D)
ASDVGKSAM

SEQ ID NO: 42 - protein, 15 aa, synthetic construct
  source 1..15: note = Seed: EYKLVVVGADGVGKS (KRAS G12D)
EYKFVVFGSDGAGKS

SEQ ID NO: 43 - protein, 24 aa, synthetic construct
  source 1..24: note = Seed: EYKLVVVGADGVGKSALTIQLIQN (KRAS G12D)
EYKFVVIGNDGAGKSALTIQLIQN

SEQ ID NO: 44 - protein, 15 aa, synthetic construct
  source 1..15: note = Seed: EYKLVVVGADGVGKS (KRAS G12D)
EYKFVVLGADGAGKS

SEQ ID NO: 45 - protein, 20 aa, synthetic construct
  source 1..20: note = Seed: MTEYKLVVVGADGVGKSALT (KRAS G12D)
MTEYKFVVSGADGIGKSALT

SEQ ID NO: 46 - protein, 20 aa, synthetic construct
  source 1..20: note = Seed: MTEYKLVVVGADGVGKSALT (KRAS G12D)
MTEYKFVVYGSDGIGKSALT

SEQ ID NO: 47 - protein, 15 aa, synthetic construct
  source 1..15: note = Seed: EYKLVVVGAVGVGKS (KRAS G12V)
EYKFVVIGRVGHGKS

SEQ ID NO: 48 - protein, 15 aa, synthetic construct
  source 1..15: note = Seed: EYKLVVVGAVGVGKS (KRAS G12V)
EYKFVVLGTVGHGKS

SEQ ID NO: 49 - protein, 15 aa, synthetic construct
  source 1..15: note = Seed: EYKLVVVGAVGVGKS (KRAS G12V)
EYKFVVYGNVGMGKS

SEQ ID NO: 50 - protein, 15 aa, synthetic construct
  source 1..15: note = Seed: EYKLVVVGAVGVGKS (KRAS G12V)
EYKIVVAGNVGIGKS

SEQ ID NO: 51 - protein, 15 aa, synthetic construct
  source 1..15: note = Seed: TEYKLVVVGAVGVGK (KRAS G12V)
TEYKIVVMGNVGYGK

SEQ ID NO: 52 - protein, 20 aa, synthetic construct
  source 1..20: note = Seed: MTEYKLVVVGARGVGKSALT (KRAS G12R)
MTEYKFVVFGSRGVGKSALT

SEQ ID NO: 53 - protein, 20 aa, synthetic construct
  source 1..20: note = Seed: MTEYKLVVVGARGVGKSALT (KRAS G12R)
MTEYKFVVIGNRGVGKSALT

SEQ ID NO: 54 - protein, 20 aa, synthetic construct
  source 1..20: note = Seed: MTEYKLVVVGARGVGKSALT (KRAS G12R)
MTEYKFVVIGVRGDGKSALT

SEQ ID NO: 55 - protein, 20 aa, synthetic construct
  source 1..20: note = Seed: MTEYKLVVVGARGVGKSALT (KRAS G12R)
MTEYKFVVMGSRGAGKSALT

SEQ ID NO: 56 - protein, 15 aa, synthetic construct
  source 1..15: note = Seed: VVVGARGVGKSALTI (KRAS G12R)
VVVIARGVPKSLLTI

SEQ ID NO: 57 - protein, 15 aa, synthetic construct
  source 1..15: note = Seed: EYKLVVVGACGVGKS (KRAS G12C)
EYKFVVFGNCGAGKS

SEQ ID NO: 58 - protein, 15 aa, synthetic construct
  source 1..15: note = Seed: EYKLVVVGACGVGKS (KRAS G12C)
EYKFVVSGACGVGKS

SEQ ID NO: 59 - protein, 15 aa, synthetic construct
  source 1..15: note = Seed: EYKLVVVGACGVGKS (KRAS G12C)
EYKFVVSGNCGLGKS

SEQ ID NO: 60 - protein, 15 aa, synthetic construct
  source 1..15: note = Seed: EYKLVVVGACGVGKS (KRAS G12C)
EYKLVVMGPCGAGKS

SEQ ID NO: 61 - protein, 15 aa, synthetic construct
  source 1..15: note = Seed: KLVVVGACGVGKSAL (KRAS G12C)
KLVIVGICKVGHSAL

SEQ ID NO: 62 - protein, 15 aa, synthetic construct
  source 1..15: note = Seed: EYKLVVVGAGDVGKS (KRAS G13D)
EYKFVVFGNGDLGKS

SEQ ID NO: 63 - protein, 15 aa, synthetic construct
  source 1..15: note = Seed: EYKLVVVGAGDVGKS (KRAS G13D)
EYKFVVMGNGDSGKS

SEQ ID NO: 64 - protein, 15 aa, synthetic construct
  source 1..15: note = Seed: EYKLVVVGAGDVGKS (KRAS G13D)
EYKFVVSGSGDVGKS

SEQ ID NO: 65 - protein, 15 aa, synthetic construct
  source 1..15: note = Seed: EYKLVVVGAGDVGKS (KRAS G13D)
EYKIVVMGRGDMGKS

SEQ ID NO: 66 - protein, 15 aa, Homo sapiens
  source 1..15: note = Native (KRAS G13D)
YKLVVVGAGDVGKSA

SEQ ID NO: 67 - protein, 9 aa, synthetic construct
  source 1..9: note = Seed: LVVVGACGV (KRAS G12C)
LLVVGACGV

SEQ ID NO: 68 - protein, 9 aa, synthetic construct
  source 1..9: note = Seed: LVVVGAVGV (KRAS G12V)
LLVVGAVGV

SEQ ID NO: 69 - protein, 9 aa, synthetic construct
  source 1..9: note = Seed: LVVVGAVGV (KRAS G12V)
LMVVGAVGI

SEQ ID NO: 70 - protein, 9 aa, synthetic construct
  source 1..9: note = Seed: LVVVGACGV (KRAS G12C)
LMVVGACGI

SEQ ID NO: 71 - protein, 9 aa, synthetic construct
  source 1..9: note = Seed: LVVVGACGV (KRAS G12C)
LLVVGACGI

SEQ ID NO: 72 - protein, 9 aa, synthetic construct
  source 1..9: note = Seed: LVVVGAVGV (KRAS G12V)
LMVVGAVGL

SEQ ID NO: 73 - protein, 9 aa, synthetic construct
  source 1..9: note = Seed: LVVVGACGV (KRAS G12C)
LMVVGACGL

SEQ ID NO: 74 - protein, 9 aa, synthetic construct
  source 1..9: note = Seed: LVVVGACGV (KRAS G12C)
LLVVGACGL

SEQ ID NO: 75 - protein, 9 aa, synthetic construct
  source 1..9: note = Seed: LVVVGACGV (KRAS G12C)
LIVVGACGV

SEQ ID NO: 76 - protein, 9 aa, synthetic construct
  source 1..9: note = Seed: LVVVGAVGV (KRAS G12V)
LLVVGAVGI

SEQ ID NO: 77 - protein, 9 aa, synthetic construct
  source 1..9: note = Seed: AGDVGKSAL (KRAS G13D)
ATDVGKSAL

SEQ ID NO: 78 - protein, 9 aa, synthetic construct
  source 1..9: note = Seed: LVVVGACGV (KRAS G12C)
LMVVGACGA

SEQ ID NO: 79 - protein, 9 aa, synthetic construct
  source 1..9: note = Seed: AGDVGKSAL (KRAS G13D)
AIDVGKSAL

SEQ ID NO: 80 - protein, 9 aa, synthetic construct
  source 1..9: note = Seed: AGDVGKSAL (KRAS G13D)
AVDVGKSAL

SEQ ID NO: 81 - protein, 9 aa, synthetic construct
  source 1..9: note = Seed: AGDVGKSAL (KRAS G13D)
AFDVGKSAL

SEQ ID NO: 82 - protein, 9 aa, synthetic construct
  source 1..9: note = Seed: AGDVGKSAL (KRAS G13D)
AADVGKSAV

SEQ ID NO: 83 - protein, 9 aa, synthetic construct
  source 1..9: note = Seed: LVVVGAVGV (KRAS G12V)
LMVVGAVGA

SEQ ID NO: 84 - protein, 9 aa, synthetic construct
  source 1..9: note = Seed: AGDVGKSAL (KRAS G13D)
AADVGKSAI

SEQ ID NO: 85 - protein, 9 aa, synthetic construct
  source 1..9: note = Seed: AGDVGKSAL (KRAS G13D)
AFDVGKSAM

SEQ ID NO: 86 - protein, 9 aa, synthetic construct
  source 1..9: note = Seed: GADGVGKSA (KRAS G12D)
GTDGVGKSL

SEQ ID NO: 87 - protein, 9 aa, synthetic construct
  source 1..9: note = Seed: AGDVGKSAL (KRAS G13D)
AYDVGKSAL

SEQ ID NO: 88 - protein, 9 aa, synthetic construct
  source 1..9: note = Seed: GADGVGKSA (KRAS G12D)
GSDGVGKSL

SEQ ID NO: 89 - protein, 9 aa, synthetic construct
  source 1..9: note = Seed: AGDVGKSAL (KRAS G13D)
ATDVGKSAM

SEQ ID NO: 90 - protein, 9 aa, synthetic construct
  source 1..9: note = Seed: LVVVGAVGV (KRAS G12V)
LLVVGAVGL

SEQ ID NO: 91 - protein, 9 aa, synthetic construct
  source 1..9: note = Seed: AGDVGKSAL (KRAS G13D)
AIDVGKSAM

SEQ ID NO: 92 - protein, 9 aa, synthetic construct
  source 1..9: note = Seed: AGDVGKSAL (KRAS G13D)
ALDVGKSAL

SEQ ID NO: 93 - protein, 9 aa, synthetic construct
  source 1..9: note = Seed: AGDVGKSAL (KRAS G13D)
AWDVGKSAL

SEQ ID NO: 94 - protein, 9 aa, synthetic construct
  source 1..9: note = Seed: LVVVGAVGV (KRAS G12V)
LIVVGAVGV

SEQ ID NO: 95 - protein, 9 aa, synthetic construct
  source 1..9: note = Seed: AGDVGKSAL (KRAS G13D)
ATDVGKSAI

SEQ ID NO: 96 - protein, 9 aa, synthetic construct
  source 1..9: note = Seed: AGDVGKSAL (KRAS G13D)
AVDVGKSAM

SEQ ID NO: 97 - protein, 9 aa, synthetic construct
  source 1..9: note = Seed: GADGVGKSA (KRAS G12D)
GIDGVGKSL

SEQ ID NO: 98 - protein, 9 aa, synthetic construct
  source 1..9: note = Seed: GADGVGKSA (KRAS G12D)
GVDGVGKSL

SEQ ID NO: 99 - protein, 9 aa, synthetic construct
  source 1..9: note = Seed: AGDVGKSAL (KRAS G13D)
ATDVGKSAV

SEQ ID NO: 100 - protein, 9 aa, synthetic construct
  source 1..9: note = Seed: AGDVGKSAL (KRAS G13D)
ASDVGKSAV

SEQ ID NO: 101 - protein, 9 aa, synthetic construct
  source 1..9: note = Seed: GARGVGKSA (KRAS G12R)
GPRGVGKSL

SEQ ID NO: 102 - protein, 9 aa, synthetic construct
  source 1..9: note = Seed: AGDVGKSAL (KRAS G13D)
ASDVGKSAI

SEQ ID NO: 103 - protein, 9 aa, synthetic construct
  source 1..9: note = Seed: AGDVGKSAL (KRAS G13D)
AFDVGKSAF

SEQ ID NO: 104 - protein, 9 aa, synthetic construct
  source 1..9: note = Seed: GADGVGKSA (KRAS G12D)
GTDGVGKSM

SEQ ID NO: 105 - protein, 9 aa, synthetic construct
  source 1..9: note = Seed: GADGVGKSA (KRAS G12D)
GSDGVGKSM

SEQ ID NO: 106 - protein, 9 aa, synthetic construct
  source 1..9: note = Seed: AGDVGKSAL (KRAS G13D)
AIDVGKSAV

SEQ ID NO: 107 - protein, 9 aa, synthetic construct
  source 1..9: note = Seed: AGDVGKSAL (KRAS G13D)
AVDVGKSAI

SEQ ID NO: 108 - protein, 9 aa, synthetic construct
  source 1..9: note = Seed: AGDVGKSAL (KRAS G13D)
AIDVGKSAI

SEQ ID NO: 109 - protein, 9 aa, synthetic construct
  source 1..9: note = Seed: LVVVGACGV (KRAS G12C)
LLVVGACGA

SEQ ID NO: 110 - protein, 9 aa, synthetic construct
  source 1..9: note = Seed: AGDVGKSAL (KRAS G13D)
AADVGKSAF

SEQ ID NO: 111 - protein, 9 aa, synthetic construct
  source 1..9: note = Seed: AGDVGKSAL (KRAS G13D)
AVDVGKSAV

SEQ ID NO: 112 - protein, 9 aa, synthetic construct
  source 1..9: note = Seed: GADGVGKSA (KRAS G12D)
GLDGVGKSL

SEQ ID NO: 113 - protein, 9 aa, synthetic construct
  source 1..9: note = Seed: GARGVGKSA (KRAS G12R)
GPRGVGKSM

SEQ ID NO: 114 - protein, 9 aa, synthetic construct
  source 1..9: note = Seed: AGDVGKSAL (KRAS G13D)
AWDVGKSAM

SEQ ID NO: 115 - protein, 9 aa, synthetic construct
  source 1..9: note = Seed: AGDVGKSAL (KRAS G13D)
ALDVGKSAM

SEQ ID NO: 116 - protein, 9 aa, synthetic construct
  source 1..9: note = Seed: GADGVGKSA (KRAS G12D)
GADGVGKSV

SEQ ID NO: 117 - protein, 9 aa, synthetic construct
  source 1..9: note = Seed: AGDVGKSAL (KRAS G13D)
AMDVGKSAL

SEQ ID NO: 118 - protein, 9 aa, synthetic construct
  source 1..9: note = Seed: GAVGVGKSA (KRAS G12V)
GAVGVGKSY

SEQ ID NO: 119 - protein, 9 aa, synthetic construct
  source 1..9: note = Seed: AGDVGKSAL (KRAS G13D)
AFDVGKSAI

SEQ ID NO: 120 - protein, 9 aa, synthetic construct
  source 1..9: note = Seed: VVGAVGVGK (KRAS G12V)
VTGAVGVGR

SEQ ID NO: 121 - protein, 9 aa, synthetic construct
  source 1..9: note = Seed: GADGVGKSA (KRAS G12D)
GIDGVGKSM

SEQ ID NO: 122 - protein, 9 aa, synthetic construct
  source 1..9: note = Seed: LVVVGAVGV (KRAS G12V)
LQVVGAVGV

SEQ ID NO: 123 - protein, 9 aa, synthetic construct
  source 1..9: note = Seed: GADGVGKSA (KRAS G12D)
GADGVGKSI

SEQ ID NO: 124 - protein, 9 aa, synthetic construct
  source 1..9: note = Seed: GADGVGKSA (KRAS G12D)
GVDGVGKSM

SEQ ID NO: 125 - protein, 9 aa, synthetic construct
  source 1..9: note = Seed: LVVVGADGV (KRAS G12D)
LMVVGADGI

SEQ ID NO: 126 - protein, 9 aa, synthetic construct
  source 1..9: note = Seed: AGDVGKSAL (KRAS G13D)
AIDVGKSAF

SEQ ID NO: 127 - protein, 9 aa, synthetic construct
  source 1..9: note = Seed: GADGVGKSA (KRAS G12D)
GADGVGKSF

SEQ ID NO: 128 - protein, 9 aa, synthetic construct
  source 1..9: note = Seed: AGDVGKSAL (KRAS G13D)
AYDVGKSAF

SEQ ID NO: 129 - protein, 9 aa, synthetic construct
  source 1..9: note = Seed: GADGVGKSA (KRAS G12D)
GLDGVGKSM

SEQ ID NO: 130 - protein, 9 aa, synthetic construct
  source 1..9: note = Seed: AGDVGKSAL (KRAS G13D)
AFDVGKSAV

SEQ ID NO: 131 - protein, 9 aa, synthetic construct
  source 1..9: note = Seed: GAVGVGKSA (KRAS G12V)
GAVGVGKSM

SEQ ID NO: 132 - protein, 9 aa, synthetic construct
  source 1..9: note = Seed: GARGVGKSA (KRAS G12R)
GPRGVGKSI

SEQ ID NO: 133 - protein, 9 aa, synthetic construct
  source 1..9: note = Seed: AGDVGKSAL (KRAS G13D)
ATDVGKSAF

SEQ ID NO: 134 - protein, 9 aa, synthetic construct
  source 1..9: note = Seed: GADGVGKSA (KRAS G12D)
GMDGVGKSL

SEQ ID NO: 135 - protein, 9 aa, synthetic construct
  source 1..9: note = Seed: GADGVGKSA (KRAS G12D)
GTDGVGKSI

SEQ ID NO: 136 - protein, 9 aa, synthetic construct
  source 1..9: note = Seed: AGDVGKSAL (KRAS G13D)
AVDVGKSAF

SEQ ID NO: 137 - protein, 9 aa, synthetic construct
  source 1..9: note = Seed: AGDVGKSAL (KRAS G13D)
AGDVGKSAM

SEQ ID NO: 138 - protein, 9 aa, synthetic construct
  source 1..9: note = Seed: GAVGVGKSA (KRAS G12V)
GPVGVGKSA

SEQ ID NO: 139 - protein, 9 aa, synthetic construct
  source 1..9: note = Seed: GADGVGKSA (KRAS G12D)
GTDGVGKSV

SEQ ID NO: 140 - protein, 9 aa, synthetic construct
  source 1..9: note = Seed: AGDVGKSAL (KRAS G13D)
ASDVGKSAF

SEQ ID NO: 141 - protein, 9 aa, synthetic construct
  source 1..9: note = Seed: GADGVGKSA (KRAS G12D)
GSDGVGKSV

SEQ ID NO: 142 - protein, 9 aa, synthetic construct
  source 1..9: note = Seed: VVGAVGVGK (KRAS G12V)
VTGAVGVGK

SEQ ID NO: 143 - protein, 9 aa, synthetic construct
  source 1..9: note = Seed: GADGVGKSA (KRAS G12D)
GSDGVGKSI

SEQ ID NO: 144 - protein, 9 aa, synthetic construct
  source 1..9: note = Seed: AGDVGKSAL (KRAS G13D)
AWDVGKSAF

SEQ ID NO: 145 - protein, 9 aa, synthetic construct
  source 1..9: note = Seed: GAGDVGKSA (KRAS G13D)
GAGDVGKSY

SEQ ID NO: 146 - protein, 9 aa, synthetic construct
  source 1..9: note = Seed: GADGVGKSA (KRAS G12D)
GSDGVGKSF

SEQ ID NO: 147 - protein, 9 aa, synthetic construct
  source 1..9: note = Seed: GADGVGKSA (KRAS G12D)
GTDGVGKSF

SEQ ID NO: 148 - protein, 9 aa, synthetic construct
  source 1..9: note = Seed: AGDVGKSAL (KRAS G13D)
AMDVGKSAM

SEQ ID NO: 149 - protein, 9 aa, synthetic construct
  source 1..9: note = Seed: GADGVGKSA (KRAS G12D)
GVDGVGKSI

SEQ ID NO: 150 - protein, 9 aa, synthetic construct
  source 1..9: note = Seed: AGDVGKSAL (KRAS G13D)
ALDVGKSAV

SEQ ID NO: 151 - protein, 9 aa, synthetic construct
  source 1..9: note = Seed: AGDVGKSAL (KRAS G13D)
AYDVGKSAI

SEQ ID NO: 152 - protein, 9 aa, synthetic construct
  source 1..9: note = Seed: GAVGVGKSA (KRAS G12V)
GAVGVGKSF

SEQ ID NO: 153 - protein, 9 aa, synthetic construct
  source 1..9: note = Seed: AGDVGKSAL (KRAS G13D)
ALDVGKSAI

SEQ ID NO: 154 - protein, 9 aa, synthetic construct
  source 1..9: note = Seed: LVVVGADGV (KRAS G12D)
LLVVGADGL

SEQ ID NO: 155 - protein, 9 aa, synthetic construct
  source 1..9: note = Seed: GADGVGKSA (KRAS G12D)
GIDGVGKSF

SEQ ID NO: 156 - protein, 9 aa, synthetic construct
  source 1..9: note = Seed: GADGVGKSA (KRAS G12D)
GMDGVGKSM

SEQ ID NO: 157 - protein, 9 aa, synthetic construct
  source 1..9: note = Seed: GADGVGKSA (KRAS G12D)
GIDGVGKSI

SEQ ID NO: 158 - protein, 9 aa, synthetic construct
  source 1..9: note = Seed: GADGVGKSA (KRAS G12D)
GVDGVGKSF

SEQ ID NO: 159 - protein, 9 aa, synthetic construct
  source 1..9: note = Seed: GADGVGKSA (KRAS G12D)
GIDGVGKSV

SEQ ID NO: 160 - protein, 9 aa, synthetic construct
  source 1..9: note = Seed: GADGVGKSA (KRAS G12D)
GVDGVGKSV

SEQ ID NO: 161 - protein, 9 aa, synthetic construct
  source 1..9: note = Seed: GAGDVGKSA (KRAS G13D)
GAGDVGKSL

SEQ ID NO: 162 - protein, 9 aa, synthetic construct
  source 1..9: note = Seed: LVVVGADGV (KRAS G12D)
LIVVGADGV

SEQ ID NO: 163 - protein, 9 aa, synthetic construct
  source 1..9: note = Seed: VVGAVGVGK (KRAS G12V)
VAGAVGVGY

SEQ ID NO: 164 - protein, 9 aa, synthetic construct
  source 1..9: note = Seed: AGDVGKSAL (KRAS G13D)
AYDVGKSAV

SEQ ID NO: 165 - protein, 9 aa, synthetic construct
  source 1..9: note = Seed: GARGVGKSA (KRAS G12R)
GARGVGKSF

SEQ ID NO: 166 - protein, 9 aa, synthetic construct
  source 1..9: note = Seed: AGDVGKSAL (KRAS G13D)
AWDVGKSAI

SEQ ID NO: 167 - protein, 9 aa, synthetic construct
  source 1..9: note = Seed: GADGVGKSA (KRAS G12D)
GGDGVGKSL

SEQ ID NO: 168 - protein, 9 aa, synthetic construct
  source 1..9: note = Seed: GARGVGKSA (KRAS G12R)
GPRGVGKSF

SEQ ID NO: 169 - protein, 9 aa, synthetic construct
  source 1..9: note = Seed: VVGAVGVGK (KRAS G12V)
VAGAVGVGL

SEQ ID NO: 170 - protein, 9 aa, synthetic construct
  source 1..9: note = Seed: GAVGVGKSA (KRAS G12V)
GSVGVGKSY

SEQ ID NO: 171 - protein, 9 aa, synthetic construct
  source 1..9: note = Seed: GACGVGKSA (KRAS G12C)
GACGVGKSY

SEQ ID NO: 172 - protein, 9 aa, synthetic construct
  source 1..9: note = Seed: AGDVGKSAL (KRAS G13D)
AGDVGKSAV

SEQ ID NO: 173 - protein, 9 aa, synthetic construct
  source 1..9: note = Seed: AGDVGKSAL (KRAS G13D)
ALDVGKSAF

SEQ ID NO: 174 - protein, 9 aa, synthetic construct
  source 1..9: note = Seed: AGDVGKSAL (KRAS G13D)
AGDVGKSAI

SEQ ID NO: 175 - protein, 9 aa, synthetic construct
  source 1..9: note = Seed: GAGDVGKSA (KRAS G13D)
GAGDVGKSF

SEQ ID NO: 176 - protein, 9 aa, synthetic construct
  source 1..9: note = Seed: GACGVGKSA (KRAS G12C)
GPCGVGKSA

SEQ ID NO: 177 - protein, 9 aa, synthetic construct
  source 1..9: note = Seed: DVGKSALTI (KRAS G13D)
DVGKSALTF

SEQ ID NO: 178 - protein, 9 aa, synthetic construct
  source 1..9: note = Seed: AGDVGKSAL (KRAS G13D)
AWDVGKSAV

SEQ ID NO: 179 - protein, 9 aa, synthetic construct
  source 1..9: note = Seed: LVVVGAVGV (KRAS G12V)
LLVVGAVGA

SEQ ID NO: 180 - protein, 9 aa, synthetic construct
  source 1..9: note = Seed: GADGVGKSA (KRAS G12D)
GYDGVGKSL

SEQ ID NO: 181 - protein, 9 aa, synthetic construct
  source 1..9: note = Seed: VVGARGVGK (KRAS G12R)
VAGARGVGL

SEQ ID NO: 182 - protein, 9 aa, synthetic construct
  source 1..9: note = Seed: GARGVGKSA (KRAS G12R)
GSRGVGKSY

SEQ ID NO: 183 - protein, 9 aa, synthetic construct
  source 1..9: note = Seed: AGDVGKSAL (KRAS G13D)
AMDVGKSAV

SEQ ID NO: 184 - protein, 9 aa, synthetic construct
  source 1..9: note = Seed: AGDVGKSAL (KRAS G13D)
AMDVGKSAI

SEQ ID NO: 185 - protein, 9 aa, synthetic construct
  source 1..9: note = Seed: DVGKSALTI (KRAS G13D)
DVGKSALTW

SEQ ID NO: 186 - protein, 9 aa, synthetic construct
  source 1..9: note = Seed: AGDVGKSAL (KRAS G13D)
ANDVGKSAL

SEQ ID NO: 187 - protein, 9 aa, synthetic construct
  source 1..9: note = Seed: GACGVGKSA (KRAS G12C)
GACGVGKSM

SEQ ID NO: 188 - protein, 9 aa, synthetic construct
  source 1..9: note = Seed: AGDVGKSAL (KRAS G13D)
AQDVGKSAL

SEQ ID NO: 189 - protein, 9 aa, synthetic construct
  source 1..9: note = Seed: GAVGVGKSA (KRAS G12V)
GSVGVGKSL

SEQ ID NO: 190 - protein, 9 aa, synthetic construct
  source 1..9: note = Seed: GADGVGKSA (KRAS G12D)
GGDGVGKSM

SEQ ID NO: 191 - protein, 9 aa, synthetic construct
  source 1..9: note = Seed: AGDVGKSAL (KRAS G13D)
AGDVGKSAF

SEQ ID NO: 192 - protein, 9 aa, synthetic construct
  source 1..9: note = Seed: GAVGVGKSA (KRAS G12V)
GTVGVGKSY

SEQ ID NO: 193 - protein, 9 aa, synthetic construct
  source 1..9: note = Seed: GADGVGKSA (KRAS G12D)
GLDGVGKSF

SEQ ID NO: 194 - protein, 9 aa, synthetic construct
  source 1..9: note = Seed: LVVVGADGV (KRAS G12D)
LQVVGADGV

SEQ ID NO: 195 - protein, 9 aa, synthetic construct
  source 1..9: note = Seed: GARGVGKSA (KRAS G12R)
GARGVGKSL

SEQ ID NO: 196 - protein, 9 aa, synthetic construct
  source 1..9: note = Seed: GAVGVGKSA (KRAS G12V)
GSVGVGKSF

SEQ ID NO: 197 - protein, 9 aa, synthetic construct
  source 1..9: note = Seed: AGDVGKSAL (KRAS G13D)
ACDVGKSAL

SEQ ID NO: 198 - protein, 9 aa, synthetic construct
  source 1..9: note = Seed: VVGAVGVGK (KRAS G12V)
VAGAVGVGM

SEQ ID NO: 199 - protein, 9 aa, synthetic construct
  source 1..9: note = Seed: AGDVGKSAL (KRAS G13D)
AEDVGKSAL

SEQ ID NO: 200 - protein, 9 aa, synthetic construct
  source 1..9: note = Seed: LVVVGADGV (KRAS G12D)
LLVVGADGI

SEQ ID NO: 201 - protein, 9 aa, synthetic construct
  source 1..9: note = Seed: AGDVGKSAL (KRAS G13D)
AEDVGKSAF

SEQ ID NO: 202 - protein, 9 aa, synthetic construct
  source 1..9: note = Seed: GADGVGKSA (KRAS G12D)
GLDGVGKSV

SEQ ID NO: 203 - protein, 9 aa, synthetic construct
  source 1..9: note = Seed: VVGAVGVGK (KRAS G12V)
VAGAVGVGR

SEQ ID NO: 204 - protein, 9 aa, synthetic construct
  source 1..9: note = Seed: VVGAVGVGK (KRAS G12V)
VAGAVGVGF

SEQ ID NO: 205 - protein, 9 aa, synthetic construct
  source 1..9: note = Seed: GADGVGKSA (KRAS G12D)
GLDGVGKSI

SEQ ID NO: 206 - protein, 9 aa, synthetic construct
  source 1..9: note = Seed: VVGACGVGK (KRAS G12C)
VTGACGVGR

SEQ ID NO: 207 - protein, 9 aa, synthetic construct
  source 1..9: note = Seed: AGDVGKSAL (KRAS G13D)
AADVGKSAY

SEQ ID NO: 208 - protein, 9 aa, synthetic construct
  source 1..9: note = Seed: GARGVGKSA (KRAS G12R)
GVRGVGKSY

SEQ ID NO: 209 - protein, 9 aa, synthetic construct
  source 1..9: note = Seed: GADGVGKSA (KRAS G12D)
GFDGVGKSL

SEQ ID NO: 210 - protein, 9 aa, synthetic construct
  source 1..9: note = Seed: GAVGVGKSA (KRAS G12V)
GVVGVGKSY

SEQ ID NO: 211 - protein, 9 aa, synthetic construct
  source 1..9: note = Seed: GARGVGKSA (KRAS G12R)
GSRGVGKSF

SEQ ID NO: 212 - protein, 9 aa, synthetic construct
  source 1..9: note = Seed: LVVVGARGV (KRAS G12R)
LMVVGARGI

SEQ ID NO: 213 - protein, 9 aa, synthetic construct
  source 1..9: note = Seed: GACGVGKSA (KRAS G12C)
GACGVGKSF

SEQ ID NO: 214 - protein, 9 aa, synthetic construct
  source 1..9: note = Seed: GAVGVGKSA (KRAS G12V)
GTVGVGKSF

SEQ ID NO: 215 - protein, 9 aa, synthetic construct
  source 1..9: note = Seed: DVGKSALTI (KRAS G13D)
DAGKSALTI

SEQ ID NO: 216 - protein, 9 aa, synthetic construct
  source 1..9: note = Seed: AGDVGKSAL (KRAS G13D)
AQDVGKSAM

SEQ ID NO: 217 - protein, 9 aa, synthetic construct
  source 1..9: note = Seed: AGDVGKSAL (KRAS G13D)
ANDVGKSAM

SEQ ID NO: 218 - protein, 9 aa, synthetic construct
  source 1..9: note = Seed: AGDVGKSAL (KRAS G13D)
AYDVGKSAY

SEQ ID NO: 219 - protein, 9 aa, synthetic construct
  source 1..9: note = Seed: GARGVGKSA (KRAS G12R)
GTRGVGKSY

SEQ ID NO: 220 - protein, 9 aa, synthetic construct
  source 1..9: note = Seed: GAGDVGKSA (KRAS G13D)
GPGDVGKSA

SEQ ID NO: 221 - protein, 9 aa, synthetic construct
  source 1..9: note = Seed: GADGVGKSA (KRAS G12D)
GMDGVGKSV

SEQ ID NO: 222 - protein, 9 aa, synthetic construct
  source 1..9: note = Seed: GARGVGKSA (KRAS G12R)
GVRGVGKSF

SEQ ID NO: 223 - protein, 9 aa, synthetic construct
  source 1..9: note = Seed: VVGAVGVGK (KRAS G12V)
VSGAVGVGR

SEQ ID NO: 224 - protein, 9 aa, synthetic construct
  source 1..9: note = Seed: GADGVGKSA (KRAS G12D)
GFDGVGKSM

SEQ ID NO: 225 - protein, 9 aa, synthetic construct
  source 1..9: note = Seed: AGDVGKSAL (KRAS G13D)
AMDVGKSAF

SEQ ID NO: 226 - protein, 9 aa, synthetic construct
  source 1..9: note = Seed: GADGVGKSA (KRAS G12D)
GCDGVGKSL

SEQ ID NO: 227 - protein, 9 aa, synthetic construct
  source 1..9: note = Seed: GAGDVGKSA (KRAS G13D)
GVGDVGKSY

SEQ ID NO: 228 - protein, 9 aa, synthetic construct
  source 1..9: note = Seed: GARGVGKSA (KRAS G12R)
GTRGVGKSF

SEQ ID NO: 229 - protein, 9 aa, synthetic construct
  source 1..9: note = Seed: GARGVGKSA (KRAS G12R)
GIRGVGKSY

SEQ ID NO: 230 - protein, 9 aa, synthetic construct
  source 1..9: note = Seed: GADGVGKSA (KRAS G12D)
GMDGVGKSI

SEQ ID NO: 231 - protein, 9 aa, synthetic construct
  source 1..9: note = Seed: AGDVGKSAL (KRAS G13D)
ACDVGKSAM

SEQ ID NO: 232 - protein, 9 aa, synthetic construct
  source 1..9: note = Seed: GAGDVGKSA (KRAS G13D)
GSGDVGKSY

SEQ ID NO: 233 - protein, 9 aa, synthetic construct
  source 1..9: note = Seed: GAVGVGKSA (KRAS G12V)
GAVGVGKSV

SEQ ID NO: 234 - protein, 9 aa, synthetic construct
  source 1..9: note = Seed: GAVGVGKSA (KRAS G12V)
GSVGVGKSM

SEQ ID NO: 235 - protein, 9 aa, synthetic construct
  source 1..9: note = Seed: DVGKSALTI (KRAS G13D)
DVGKSALTY

SEQ ID NO: 236 - protein, 9 aa, synthetic construct
  source 1..9: note = Seed: GAVGVGKSA (KRAS G12V)
GVVGVGKSF

SEQ ID NO: 237 - protein, 9 aa, synthetic construct
  source 1..9: note = Seed: GAVGVGKSA (KRAS G12V)
GIVGVGKSY

SEQ ID NO: 238 - protein, 9 aa, synthetic construct
  source 1..9: note = Seed: VVGACGVGK (KRAS G12C)
VAGACGVGY

SEQ ID NO: 239 - protein, 9 aa, synthetic construct
  source 1..9: note = Seed: GACGVGKSA (KRAS G12C)
GSCGVGKSY

SEQ ID NO: 240 - protein, 9 aa, synthetic construct
  source 1..9: note = Seed: GAVGVGKSA (KRAS G12V)
GTVGVGKSL

SEQ ID NO: 241 - protein, 9 aa, synthetic construct
  source 1..9: note = Seed: GAVGVGKSA (KRAS G12V)
GAVGVGKSH

SEQ ID NO: 242 - protein, 9 aa, synthetic construct
  source 1..9: note = Seed: GADGVGKSA (KRAS G12D)
GMDGVGKSF

SEQ ID NO: 243 - protein, 9 aa, synthetic construct
  source 1..9: note = Seed: GADGVGKSA (KRAS G12D)
GQDGVGKSL

SEQ ID NO: 244 - protein, 9 aa, synthetic construct
  source 1..9: note = Seed: GADGVGKSA (KRAS G12D)
GCDGVGKSM

SEQ ID NO: 245 - protein, 9 aa, synthetic construct
  source 1..9: note = Seed: GAGDVGKSA (KRAS G13D)
GSGDVGKSF

SEQ ID NO: 246 - protein, 9 aa, synthetic construct
  source 1..9: note = Seed: GAGDVGKSA (KRAS G13D)
GTGDVGKSY

SEQ ID NO: 247 - protein, 9 aa, synthetic construct
  source 1..9: note = Seed: GADGVGKSA (KRAS G12D)
GGDGVGKSI

SEQ ID NO: 248 - protein, 9 aa, synthetic construct
  source 1..9: note = Seed: AGDVGKSAL (KRAS G13D)
ACDVGKSAV

SEQ ID NO: 249 - protein, 9 aa, synthetic construct
  source 1..9: note = Seed: GARGVGKSA (KRAS G12R)
GSRGVGKSM

SEQ ID NO: 250 - protein, 9 aa, synthetic construct
  source 1..9: note = Seed: VVGAVGVGK (KRAS G12V)
VSGAVGVGK

SEQ ID NO: 251 - protein, 9 aa, synthetic construct
  source 1..9: note = Seed: GADGVGKSA (KRAS G12D)
GYDGVGKSF

SEQ ID NO: 252 - protein, 9 aa, synthetic construct
  source 1..9: note = Seed: DVGKSALTI (KRAS G13D)
DTGKSALTF

SEQ ID NO: 253 - protein, 9 aa, synthetic construct
  source 1..9: note = Seed: GARGVGKSA (KRAS G12R)
GPRGVGKSP

SEQ ID NO: 254 - protein, 9 aa, synthetic construct
  source 1..9: note = Seed: GAVGVGKSA (KRAS G12V)
GPVGVGKSL

SEQ ID NO: 255 - protein, 9 aa, synthetic construct
  source 1..9: note = Seed: GADGVGKSA (KRAS G12D)
GGDGVGKSF

SEQ ID NO: 256 - protein, 9 aa, synthetic construct
  source 1..9: note = Seed: GACGVGKSA (KRAS G12C)
GPCGVGKSV

SEQ ID NO: 257 - protein, 9 aa, synthetic construct
  source 1..9: note = Seed: GAGDVGKSA (KRAS G13D)
GVGDVGKSF

SEQ ID NO: 258 - protein, 9 aa, synthetic construct
  source 1..9: note = Seed: GAGDVGKSA (KRAS G13D)
GIGDVGKSY

SEQ ID NO: 259 - protein, 9 aa, synthetic construct
  source 1..9: note = Seed: GAVGVGKSA (KRAS G12V)
GGVGVGKSY

SEQ ID NO: 260 - protein, 9 aa, synthetic construct
  source 1..9: note = Seed: GARGVGKSA (KRAS G12R)
GPRGVGKST

SEQ ID NO: 261 - protein, 9 aa, synthetic construct
  source 1..9: note = Seed: GADGVGKSA (KRAS G12D)
GGDGVGKSV

SEQ ID NO: 262 - protein, 9 aa, synthetic construct
  source 1..9: note = Seed: AGDVGKSAL (KRAS G13D)
ACDVGKSAF

SEQ ID NO: 263 - protein, 9 aa, synthetic construct
  source 1..9: note = Seed: GAVGVGKSA (KRAS G12V)
GAVGVGKSI

SEQ ID NO: 264 - protein, 9 aa, synthetic construct
  source 1..9: note = Seed: GARGVGKSA (KRAS G12R)
GIRGVGKSF

SEQ ID NO: 265 - protein, 9 aa, synthetic construct
  source 1..9: note = Seed: GAGDVGKSA (KRAS G13D)
GTGDVGKSF

SEQ ID NO: 266 - protein, 9 aa, synthetic construct
  source 1..9: note = Seed: GADGVGKSA (KRAS G12D)
GNDGVGKSL

SEQ ID NO: 267 - protein, 9 aa, synthetic construct
  source 1..9: note = Seed: VVGARGVGK (KRAS G12R)
VAGARGVGF

SEQ ID NO: 268 - protein, 9 aa, synthetic construct
  source 1..9: note = Seed: GACGVGKSA (KRAS G12C)
GVCGVGKSY

SEQ ID NO: 269 - protein, 9 aa, synthetic construct
  source 1..9: note = Seed: GARGVGKSA (KRAS G12R)
GVRGVGKSM

SEQ ID NO: 270 - protein, 9 aa, synthetic construct
  source 1..9: note = Seed: VVGARGVGK (KRAS G12R)
VTGARGVGR

SEQ ID NO: 271 - protein, 9 aa, synthetic construct
  source 1..9: note = Seed: VVGAVGVGK (KRAS G12V)
VAGAVGVGK

SEQ ID NO: 272 - protein, 9 aa, synthetic construct
  source 1..9: note = Seed: VVGADGVGK (KRAS G12D)
VAGADGVGY

SEQ ID NO: 273 - protein, 9 aa, synthetic construct
  source 1..9: note = Seed: GADGVGKSA (KRAS G12D)
GQDGVGKSM

SEQ ID NO: 274 - protein, 9 aa, synthetic construct
  source 1..9: note = Seed: GAVGVGKSA (KRAS G12V)
GPVGVGKST

SEQ ID NO: 275 - protein, 9 aa, synthetic construct
  source 1..9: note = Seed: AGDVGKSAL (KRAS G13D)
AEDVGKSAI

SEQ ID NO: 276 - protein, 9 aa, synthetic construct
  source 1..9: note = Seed: VVGAVGVGK (KRAS G12V)
VIGAVGVGK

SEQ ID NO: 277 - protein, 9 aa, synthetic construct
  source 1..9: note = Seed: GAVGVGKSA (KRAS G12V)
GVVGVGKSL

SEQ ID NO: 278 - protein, 9 aa, synthetic construct
  source 1..9: note = Seed: GAVGVGKSA (KRAS G12V)
GVVGVGKSM

SEQ ID NO: 279 - protein, 9 aa, synthetic construct
  source 1..9: note = Seed: VVGARGVGK (KRAS G12R)
VAGARGVGY

SEQ ID NO: 280 - protein, 9 aa, synthetic construct
  source 1..9: note = Seed: GAVGVGKSA (KRAS G12V)
GTVGVGKSM

SEQ ID NO: 281 - protein, 9 aa, synthetic construct
  source 1..9: note = Seed: AGDVGKSAL (KRAS G13D)
ACDVGKSAI

SEQ ID NO: 282 - protein, 9 aa, synthetic construct
  source 1..9: note = Seed: GARGVGKSA (KRAS G12R)
GPRGVGKSC

SEQ ID NO: 283 - protein, 9 aa, synthetic construct
  source 1..9: note = Seed: LVVVGARGV (KRAS G12R)
LLVVGARGI

SEQ ID NO: 284 - protein, 9 aa, synthetic construct
  source 1..9: note = Seed: GARGVGKSA (KRAS G12R)
GVRGVGKSL

SEQ ID NO: 285 - protein, 9 aa, synthetic construct
  source 1..9: note = Seed: GACGVGKSA (KRAS G12C)
GTCGVGKSY

SEQ ID NO: 286 - protein, 9 aa, synthetic construct
  source 1..9: note = Seed: GAVGVGKSA (KRAS G12V)
GPVGVGKSP

SEQ ID NO: 287 - protein, 9 aa, synthetic construct
  source 1..9: note = Seed: VVGAVGVGK (KRAS G12V)
VSGAVGVGY

SEQ ID NO: 288 - protein, 9 aa, synthetic construct
  source 1..9: note = Seed: GADGVGKSA (KRAS G12D)
GFDGVGKSV

SEQ ID NO: 289 - protein, 9 aa, synthetic construct
  source 1..9: note = Seed: DVGKSALTI (KRAS G13D)
DIGKSALTW

SEQ ID NO: 290 - protein, 9 aa, synthetic construct
  source 1..9: note = Seed: GADGVGKSA (KRAS G12D)
GYDGVGKSI

SEQ ID NO: 291 - protein, 9 aa, synthetic construct
  source 1..9: note = Seed: GARGVGKSA (KRAS G12R)
GARGVGKSV

SEQ ID NO: 292 - protein, 9 aa, synthetic construct
  source 1..9: note = Seed: GADGVGKSA (KRAS G12D)
GPDGVGKSA

SEQ ID NO: 293 - protein, 9 aa, synthetic construct
  source 1..9: note = Seed: GARGVGKSA (KRAS G12R)
GGRGVGKSY

SEQ ID NO: 294 - protein, 9 aa, synthetic construct
  source 1..9: note = Seed: GARGVGKSA (KRAS G12R)
GTRGVGKSM

SEQ ID NO: 295 - protein, 9 aa, synthetic construct
  source 1..9: note = Seed: GAVGVGKSA (KRAS G12V)
GIVGVGKSF

SEQ ID NO: 296 - protein, 9 aa, synthetic construct
  source 1..9: note = Seed: AGDVGKSAL (KRAS G13D)
AEDVGKSAV

SEQ ID NO: 297 - protein, 9 aa, synthetic construct
  source 1..9: note = Seed: GAVGVGKSA (KRAS G12V)
GPVGVGKSI

SEQ ID NO: 298 - protein, 9 aa, synthetic construct
  source 1..9: note = Seed: AGDVGKSAL (KRAS G13D)
ADDVGKSAF

SEQ ID NO: 299 - protein, 9 aa, synthetic construct
  source 1..9: note = Seed: GACGVGKSA (KRAS G12C)
GSCGVGKSF

SEQ ID NO: 300 - protein, 9 aa, synthetic construct
  source 1..9: note = Seed: AGDVGKSAL (KRAS G13D)
ARDVGKSAL

SEQ ID NO: 301 - protein, 9 aa, synthetic construct
  source 1..9: note = Seed: GAGDVGKSA (KRAS G13D)
GSGDVGKSM

SEQ ID NO: 302 - protein, 9 aa, synthetic construct
  source 1..9: note = Seed: AGDVGKSAL (KRAS G13D)
AQDVGKSAI

SEQ ID NO: 303 - protein, 9 aa, synthetic construct
  source 1..9: note = Seed: GAGDVGKSA (KRAS G13D)
GVGDVGKSM

SEQ ID NO: 304 - protein, 9 aa, synthetic construct
  source 1..9: note = Seed: GADGVGKSA (KRAS G12D)
GFDGVGKSF

SEQ ID NO: 305 - protein, 9 aa, synthetic construct
  source 1..9: note = Seed: GARGVGKSA (KRAS G12R)
GSRGVGKSL

SEQ ID NO: 306 - protein, 9 aa, synthetic construct
  source 1..9: note = Seed: AGDVGKSAL (KRAS G13D)
ANDVGKSAI

SEQ ID NO: 307 - protein, 9 aa, synthetic construct
  source 1..9: note = Seed: GAVGVGKSA (KRAS G12V)
GPVGVGKSM

SEQ ID NO: 308 - protein, 9 aa, synthetic construct
  source 1..9: note = Seed: GADGVGKSA (KRAS G12D)
GNDGVGKSM

SEQ ID NO: 309 - protein, 9 aa, synthetic construct
  source 1..9: note = Seed: VVGAVGVGK (KRAS G12V)
VLGAVGVGR

SEQ ID NO: 310 - protein, 9 aa, synthetic construct
  source 1..9: note = Seed: LVVVGARGV (KRAS G12R)
LMVVGARGL

SEQ ID NO: 311 - protein, 9 aa, synthetic construct
  source 1..9: note = Seed: AGDVGKSAL (KRAS G13D)
ANDVGKSAV

SEQ ID NO: 312 - protein, 9 aa, synthetic construct
  source 1..9: note = Seed: GADGVGKSA (KRAS G12D)
GWDGVGKSL

SEQ ID NO: 313 - protein, 9 aa, synthetic construct
  source 1..9: note = Seed: VVGACGVGK (KRAS G12C)
VAGACGVGR

SEQ ID NO: 314 - protein, 9 aa, synthetic construct
  source 1..9: note = Seed: AGDVGKSAL (KRAS G13D)
ADDVGKSAL

SEQ ID NO: 315 - protein, 9 aa, synthetic construct
  source 1..9: note = Seed: GACGVGKSA (KRAS G12C)
GSCGVGKSL

SEQ ID NO: 316 - protein, 9 aa, synthetic construct
  source 1..9: note = Seed: DVGKSALTI (KRAS G13D)
DAGKSALTL

SEQ ID NO: 317 - protein, 9 aa, synthetic construct
  source 1..9: note = Seed: AGDVGKSAL (KRAS G13D)
AQDVGKSAV

SEQ ID NO: 318 - protein, 9 aa, synthetic construct
  source 1..9: note = Seed: VVGAGDVGK (KRAS G13D)
VTGAGDVGR

SEQ ID NO: 319 - protein, 9 aa, synthetic construct
  source 1..9: note = Seed: VVGADGVGK (KRAS G12D)
VTGADGVGR

SEQ ID NO: 320 - protein, 9 aa, synthetic construct
  source 1..9: note = Seed: VVGAVGVGK (KRAS G12V)
VTGAVGVGY

SEQ ID NO: 321 - protein, 9 aa, synthetic construct
  source 1..9: note = Seed: VVGAVGVGK (KRAS G12V)
VVGAVGVGY

SEQ ID NO: 322 - protein, 9 aa, synthetic construct
  source 1..9: note = Seed: VVGAVGVGK (KRAS G12V)
VIGAVGVGR

SEQ ID NO: 323 - protein, 9 aa, synthetic construct
  source 1..9: note = Seed: GACGVGKSA (KRAS G12C)
GICGVGKSY

SEQ ID NO: 324 - protein, 9 aa, synthetic construct
  source 1..9: note = Seed: VVGARGVGK (KRAS G12R)
VVGARGVGR

SEQ ID NO: 325 - protein, 9 aa, synthetic construct
  source 1..9: note = Seed: GAGDVGKSA (KRAS G13D)
GIGDVGKSF

SEQ ID NO: 326 - protein, 9 aa, synthetic construct
  source 1..9: note = Seed: VVGARGVGK (KRAS G12R)
VMGARGVGR

SEQ ID NO: 327 - protein, 9 aa, synthetic construct
  source 1..9: note = Seed: VVGACGVGK (KRAS G12C)
VAGACGVGF

SEQ ID NO: 328 - protein, 9 aa, synthetic construct
  source 1..9: note = Seed: AGDVGKSAL (KRAS G13D)
AQDVGKSAF

SEQ ID NO: 329 - protein, 9 aa, synthetic construct
  source 1..9: note = Seed: AGDVGKSAL (KRAS G13D)
AVDVGKSAY

SEQ ID NO: 330 - protein, 9 aa, synthetic construct
  source 1..9: note = Seed: VVGAGDVGK (KRAS G13D)
VAGAGDVGY

SEQ ID NO: 331 - protein, 9 aa, synthetic construct
  source 1..9: note = Seed: DVGKSALTI (KRAS G13D)
DPGKSALTV

SEQ ID NO: 332 - protein, 9 aa, synthetic construct
  source 1..9: note = Seed: GADGVGKSA (KRAS G12D)
GCDGVGKSV

SEQ ID NO: 333 - protein, 9 aa, synthetic construct
  source 1..9: note = Seed: GADGVGKSA (KRAS G12D)
GFDGVGKSI

SEQ ID NO: 334 - protein, 9 aa, synthetic construct
  source 1..9: note = Seed: GARGVGKSA (KRAS G12R)
GIRGVGKSM

SEQ ID NO: 335 - protein, 9 aa, synthetic construct
  source 1..9: note = Seed: GADGVGKSA (KRAS G12D)
GCDGVGKSF

SEQ ID NO: 336 - protein, 9 aa, synthetic construct
  source 1..9: note = Seed: VVGACGVGK (KRAS G12C)
VAGACGVGL

SEQ ID NO: 337 - protein, 9 aa, synthetic construct
  source 1..9: note = Seed: LVVVGACGV (KRAS G12C)
LVVVGACGI

SEQ ID NO: 338 - protein, 9 aa, synthetic construct
  source 1..9: note = Seed: AGDVGKSAL (KRAS G13D)
ASDVGKSAY

SEQ ID NO: 339 - protein, 9 aa, synthetic construct
  source 1..9: note = Seed: GADGVGKSA (KRAS G12D)
GYDGVGKSV

SEQ ID NO: 340 - protein, 9 aa, synthetic construct
  source 1..9: note = Seed: AGDVGKSAL (KRAS G13D)
ADDVGKSAM

SEQ ID NO: 341 - protein, 9 aa, synthetic construct
  source 1..9: note = Seed: DVGKSALTI (KRAS G13D)
DTGKSALTW

SEQ ID NO: 342 - protein, 9 aa, synthetic construct
  source 1..9: note = Seed: GACGVGKSA (KRAS G12C)
GPCGVGKSL

SEQ ID NO: 343 - protein, 9 aa, synthetic construct
  source 1..9: note = Seed: GADGVGKSA (KRAS G12D)
GRDGVGKSL

SEQ ID NO: 344 - protein, 9 aa, synthetic construct
  source 1..9: note = Seed: GACGVGKSA (KRAS G12C)
GVCGVGKSF

SEQ ID NO: 345 - protein, 9 aa, synthetic construct
  source 1..9: note = Seed: GARGVGKSA (KRAS G12R)
GLRGVGKSY

SEQ ID NO: 346 - protein, 9 aa, synthetic construct
  source 1..9: note = Seed: GACGVGKSA (KRAS G12C)
GACGVGKSV

SEQ ID NO: 347 - protein, 9 aa, synthetic construct
  source 1..9: note = Seed: GACGVGKSA (KRAS G12C)
GTCGVGKSF

SEQ ID NO: 348 - protein, 9 aa, synthetic construct
  source 1..9: note = Seed: GADGVGKSA (KRAS G12D)
GSDGVGKSY

SEQ ID NO: 349 - protein, 9 aa, synthetic construct
  source 1..9: note = Seed: GACGVGKSA (KRAS G12C)
GACGVGKSI

SEQ ID NO: 350 - protein, 9 aa, synthetic construct
  source 1..9: note = Seed: GAGDVGKSA (KRAS G13D)
GTGDVGKSM

SEQ ID NO: 351 - protein, 9 aa, synthetic construct
  source 1..9: note = Seed: VVGARGVGK (KRAS G12R)
VSGARGVGM

SEQ ID NO: 352 - protein, 9 aa, synthetic construct
  source 1..9: note = Seed: VVGADGVGK (KRAS G12D)
VTGADGVGK

SEQ ID NO: 353 - protein, 9 aa, synthetic construct
  source 1..9: note = Seed: VVGAVGVGK (KRAS G12V)
VLGAVGVGK

SEQ ID NO: 354 - protein, 9 aa, synthetic construct
  source 1..9: note = Seed: AGDVGKSAL (KRAS G13D)
ARDVGKSAY

SEQ ID NO: 355 - protein, 9 aa, synthetic construct
  source 1..9: note = Seed: GAGDVGKSA (KRAS G13D)
GSGDVGKSL

SEQ ID NO: 356 - protein, 9 aa, synthetic construct
  source 1..9: note = Seed: VVGAGDVGK (KRAS G13D)
VVGAGDVGR

SEQ ID NO: 357 - protein, 9 aa, synthetic construct
  source 1..9: note = Seed: GAGDVGKSA (KRAS G13D)
GPGDVGKSV

SEQ ID NO: 358 - protein, 9 aa, synthetic construct
  source 1..9: note = Seed: DVGKSALTI (KRAS G13D)
DTGKSALTY

SEQ ID NO: 359 - protein, 9 aa, synthetic construct
  source 1..9: note = Seed: AGDVGKSAL (KRAS G13D)
ARDVGKSAF

SEQ ID NO: 360 - protein, 9 aa, synthetic construct
  source 1..9: note = Seed: GACGVGKSA (KRAS G12C)
GSCGVGKSM

SEQ ID NO: 361 - protein, 9 aa, synthetic construct
  source 1..9: note = Seed: AGDVGKSAL (KRAS G13D)
ATDVGKSAY

SEQ ID NO: 362 - protein, 9 aa, synthetic construct
  source 1..9: note = Seed: GARGVGKSA (KRAS G12R)
GMRGVGKSY

SEQ ID NO: 363 - protein, 9 aa, synthetic construct
  source 1..9: note = Seed: GAGDVGKSA (KRAS G13D)
GAGDVGKSH

SEQ ID NO: 364 - protein, 9 aa, synthetic construct
  source 1..9: note = Seed: GADGVGKSA (KRAS G12D)
GCDGVGKSI

SEQ ID NO: 365 - protein, 9 aa, synthetic construct
  source 1..9: note = Seed: GARGVGKSA (KRAS G12R)
GARGVGKSI

SEQ ID NO: 366 - protein, 9 aa, synthetic construct
  source 1..9: note = Seed: VVGADGVGK (KRAS G12D)
VVGADGVGR

SEQ ID NO: 367 - protein, 9 aa, synthetic construct
  source 1..9: note = Seed: AGDVGKSAL (KRAS G13D)
ANDVGKSAF

SEQ ID NO: 368 - protein, 9 aa, synthetic construct
  source 1..9: note = Seed: VVGACGVGK (KRAS G12C)
VSGACGVGK

SEQ ID NO: 369 - protein, 9 aa, synthetic construct
  source 1..9: note = Seed: VVGAVGVGK (KRAS G12V)
VSGAVGVGF

SEQ ID NO: 370 - protein, 9 aa, synthetic construct
  source 1..9: note = Seed: GADGVGKSA (KRAS G12D)
GWDGVGKSM

SEQ ID NO: 371 - protein, 9 aa, synthetic construct
  source 1..9: note = Seed: GARGVGKSA (KRAS G12R)
GTRGVGKSL

SEQ ID NO: 372 - protein, 9 aa, synthetic construct
  source 1..9: note = Seed: GAVGVGKSA (KRAS G12V)
GIVGVGKSM

SEQ ID NO: 373 - protein, 9 aa, synthetic construct
  source 1..9: note = Seed: VVGACGVGK (KRAS G12C)
VIGACGVGK

SEQ ID NO: 374 - protein, 9 aa, synthetic construct
  source 1..9: note = Seed: GAGDVGKSA (KRAS G13D)
GAGDVGKSV

SEQ ID NO: 375 - protein, 9 aa, synthetic construct
  source 1..9: note = Seed: GAGDVGKSA (KRAS G13D)
GGGDVGKSY

SEQ ID NO: 376 - protein, 9 aa, synthetic construct
  source 1..9: note = Seed: LVVVGACGV (KRAS G12C)
LQVVGACGL

SEQ ID NO: 377 - protein, 9 aa, synthetic construct
  source 1..9: note = Seed: GAGDVGKSA (KRAS G13D)
GVGDVGKSL

SEQ ID NO: 378 - protein, 9 aa, synthetic construct
  source 1..9: note = Seed: VVGADGVGK (KRAS G12D)
VAGADGVGF

SEQ ID NO: 379 - protein, 9 aa, synthetic construct
  source 1..9: note = Seed: GARGVGKSA (KRAS G12R)
GARGVGKSH

SEQ ID NO: 380 - protein, 9 aa, synthetic construct
  source 1..9: note = Seed: GADGVGKSA (KRAS G12D)
GVDGVGKSY

SEQ ID NO: 381 - protein, 9 aa, synthetic construct
  source 1..9: note = Seed: VVGACGVGK (KRAS G12C)
VAGACGVGM

SEQ ID NO: 382 - protein, 9 aa, synthetic construct
  source 1..9: note = Seed: GACGVGKSA (KRAS G12C)
GPCGVGKSM

SEQ ID NO: 383 - protein, 9 aa, synthetic construct
  source 1..9: note = Seed: VVGADGVGK (KRAS G12D)
VTGADGVGY

SEQ ID NO: 384 - protein, 9 aa, synthetic construct
  source 1..9: note = Seed: AGDVGKSAL (KRAS G13D)
AADVGKSAA

SEQ ID NO: 385 - protein, 9 aa, synthetic construct
  source 1..9: note = Seed: GACGVGKSA (KRAS G12C)
GGCGVGKSY

SEQ ID NO: 386 - protein, 9 aa, synthetic construct
  source 1..9: note = Seed: GAVGVGKSA (KRAS G12V)
GSVGVGKSH

SEQ ID NO: 387 - protein, 9 aa, synthetic construct
  source 1..9: note = Seed: AGDVGKSAL (KRAS G13D)
AADVGKSAC

SEQ ID NO: 388 - protein, 9 aa, synthetic construct
  source 1..9: note = Seed: DVGKSALTI (KRAS G13D)
DIGKSALTF

SEQ ID NO: 389 - protein, 9 aa, synthetic construct
  source 1..9: note = Seed: VVGAVGVGK (KRAS G12V)
VAGAVGVGH

SEQ ID NO: 390 - protein, 9 aa, synthetic construct
  source 1..9: note = Seed: GARGVGKSA (KRAS G12R)
GQRGVGKSY

SEQ ID NO: 391 - protein, 9 aa, synthetic construct
  source 1..9: note = Seed: AGDVGKSAL (KRAS G13D)
AFDVGKSAY

SEQ ID NO: 392 - protein, 9 aa, synthetic construct
  source 1..9: note = Seed: VVGARGVGK (KRAS G12R)
VSGARGVGL

SEQ ID NO: 393 - protein, 9 aa, synthetic construct
  source 1..9: note = Seed: GACGVGKSA (KRAS G12C)
GPCGVGKSI

SEQ ID NO: 394 - protein, 9 aa, synthetic construct
  source 1..9: note = Seed: GAVGVGKSA (KRAS G12V)
GQVGVGKSY

SEQ ID NO: 395 - protein, 9 aa, synthetic construct
  source 1..9: note = Seed: VVGACGVGK (KRAS G12C)
VTGACGVGY

SEQ ID NO: 396 - protein, 9 aa, synthetic construct
  source 1..9: note = Seed: GACGVGKSA (KRAS G12C)
GPCGVGKSP

SEQ ID NO: 397 - protein, 9 aa, synthetic construct
  source 1..9: note = Seed: VVGACGVGK (KRAS G12C)
VSGACGVGR

SEQ ID NO: 398 - protein, 9 aa, synthetic construct
  source 1..9: note = Seed: GAGDVGKSA (KRAS G13D)
GIGDVGKSM

SEQ ID NO: 399 - protein, 9 aa, synthetic construct
  source 1..9: note = Seed: LVVVGAVGV (KRAS G12V)
LMVVGAVGM

SEQ ID NO: 400 - protein, 9 aa, synthetic construct
  source 1..9: note = Seed: AGDVGKSAL (KRAS G13D)
ARDVGKSAM

SEQ ID NO: 401 - protein, 9 aa, synthetic construct
  source 1..9: note = Seed: LVVVGAVGV (KRAS G12V)
LQVVGAVGL

SEQ ID NO: 402 - protein, 9 aa, synthetic construct
  source 1..9: note = Seed: GACGVGKSA (KRAS G12C)
GACGVGKSH

SEQ ID NO: 403 - protein, 9 aa, synthetic construct
  source 1..9: note = Seed: GACGVGKSA (KRAS G12C)
GVCGVGKSM

SEQ ID NO: 404 - protein, 9 aa, synthetic construct
  source 1..9: note = Seed: LVVVGARGV (KRAS G12R)
LLVVGARGL

SEQ ID NO: 405 - protein, 9 aa, synthetic construct
  source 1..9: note = Seed: GACGVGKSA (KRAS G12C)
GTCGVGKSL

SEQ ID NO: 406 - protein, 9 aa, synthetic construct
  source 1..9: note = Seed: AGDVGKSAL (KRAS G13D)
AFDVGKSAC

SEQ ID NO: 407 - protein, 9 aa, synthetic construct
  source 1..9: note = Seed: GAVGVGKSA (KRAS G12V)
GLVGVGKSY

SEQ ID NO: 408 - protein, 9 aa, synthetic construct
  source 1..9: note = Seed: GAVGVGKSA (KRAS G12V)
GMVGVGKSY

SEQ ID NO: 409 - protein, 9 aa, synthetic construct
  source 1..9: note = Seed: VVGARGVGK (KRAS G12R)
VSGARGVGF

SEQ ID NO: 410 - protein, 9 aa, synthetic construct
  source 1..9: note = Seed: VVGACGVGK (KRAS G12C)
VLGACGVGK

SEQ ID NO: 411 - protein, 9 aa, synthetic construct
  source 1..9: note = Seed: GACGVGKSA (KRAS G12C)
GTCGVGKSM

SEQ ID NO: 412 - protein, 9 aa, synthetic construct
  source 1..9: note = Seed: GADGVGKSA (KRAS G12D)
GTDGVGKSY

SEQ ID NO: 413 - protein, 9 aa, synthetic construct
  source 1..9: note = Seed: GAVGVGKSA (KRAS G12V)
GGVGVGKSF

SEQ ID NO: 414 - protein, 9 aa, synthetic construct
  source 1..9: note = Seed: GAGDVGKSA (KRAS G13D)
GTGDVGKSL

SEQ ID NO: 415 - protein, 9 aa, synthetic construct
  source 1..9: note = Seed: LVVVGACGV (KRAS G12C)
LIVVGACGI

SEQ ID NO: 416 - protein, 9 aa, synthetic construct
  source 1..9: note = Seed: VVGARGVGK (KRAS G12R)
VLGARGVGK

SEQ ID NO: 417 - protein, 9 aa, synthetic construct
  source 1..9: note = Seed: LVVVGARGV (KRAS G12R)
LMVVGARGA

SEQ ID NO: 418 - protein, 9 aa, synthetic construct
  source 1..9: note = Seed: GACGVGKSA (KRAS G12C)
GPCGVGKST

SEQ ID NO: 419 - protein, 9 aa, synthetic construct
  source 1..9: note = Seed: VVGAVGVGK (KRAS G12V)
VIGAVGVGY

SEQ ID NO: 420 - protein, 9 aa, synthetic construct
  source 1..9: note = Seed: GARGVGKSA (KRAS G12R)
GPRGVGKSS

SEQ ID NO: 421 - protein, 9 aa, synthetic construct
  source 1..9: note = Seed: LVVVGACGV (KRAS G12C)
LAVVGACGV

SEQ ID NO: 422 - protein, 9 aa, synthetic construct
  source 1..9: note = Seed: GARGVGKSA (KRAS G12R)
GIRGVGKSL

SEQ ID NO: 423 - protein, 9 aa, synthetic construct
  source 1..9: note = Seed: AGDVGKSAL (KRAS G13D)
AYDVGKSAC

SEQ ID NO: 424 - protein, 9 aa, synthetic construct
  source 1..9: note = Seed: LVVVGADGV (KRAS G12D)
LMVVGADGA

SEQ ID NO: 425 - protein, 9 aa, synthetic construct
  source 1..9: note = Seed: DVGKSALTI (KRAS G13D)
DPGKSALTI

SEQ ID NO: 426 - protein, 9 aa, synthetic construct
  source 1..9: note = Seed: VVGAGDVGK (KRAS G13D)
VTGAGDVGK

SEQ ID NO: 427 - protein, 9 aa, synthetic construct
  source 1..9: note = Seed: VVGACGVGK (KRAS G12C)
VAGACGVGK

SEQ ID NO: 428 - protein, 9 aa, synthetic construct
  source 1..9: note = Seed: GADGVGKSA (KRAS G12D)
GADGVGKSC

SEQ ID NO: 429 - protein, 9 aa, synthetic construct
  source 1..9: note = Seed: GACGVGKSA (KRAS G12C)
GICGVGKSF

SEQ ID NO: 430 - protein, 9 aa, synthetic construct
  source 1..9: note = Seed: VVGAVGVGK (KRAS G12V)
VVGAVGVGF

SEQ ID NO: 431 - protein, 9 aa, synthetic construct
  source 1..9: note = Seed: VVGADGVGK (KRAS G12D)
VAGADGVGL

SEQ ID NO: 432 - protein, 9 aa, synthetic construct
  source 1..9: note = Seed: VVGARGVGK (KRAS G12R)
VSGARGVGY

SEQ ID NO: 433 - protein, 9 aa, synthetic construct
  source 1..9: note = Seed: LVVVGACGV (KRAS G12C)
LVVVGACGL

SEQ ID NO: 434 - protein, 9 aa, synthetic construct
  source 1..9: note = Seed: AGDVGKSAL (KRAS G13D)
AHDVGKSAL

SEQ ID NO: 435 - protein, 9 aa, synthetic construct
  source 1..9: note = Seed: GADGVGKSA (KRAS G12D)
GRDGVGKSM

SEQ ID NO: 436 - protein, 9 aa, synthetic construct
  source 1..9: note = Seed: VVGAGDVGK (KRAS G13D)
VTGAGDVGY

SEQ ID NO: 437 - protein, 9 aa, synthetic construct
  source 1..9: note = Seed: LVVVGACGV (KRAS G12C)
LTVVGACGI

SEQ ID NO: 438 - protein, 9 aa, synthetic construct
  source 1..9: note = Seed: VVGARGVGK (KRAS G12R)
VTGARGVGM

SEQ ID NO: 439 - protein, 9 aa, synthetic construct
  source 1..9: note = Seed: AGDVGKSAL (KRAS G13D)
APDVGKSAL

SEQ ID NO: 440 - protein, 9 aa, synthetic construct
  source 1..9: note = Seed: GACGVGKSA (KRAS G12C)
GVCGVGKSL

SEQ ID NO: 441 - protein, 9 aa, synthetic construct
  source 1..9: note = Seed: VVGAGDVGK (KRAS G13D)
VAGAGDVGF

SEQ ID NO: 442 - protein, 9 aa, synthetic construct
  source 1..9: note = Seed: GAVGVGKSA (KRAS G12V)
GIVGVGKSL

SEQ ID NO: 443 - protein, 9 aa, synthetic construct
  source 1..9: note = Seed: VVGAGDVGK (KRAS G13D)
VAGAGDVGL

SEQ ID NO: 444 - protein, 9 aa, synthetic construct
  source 1..9: note = Seed: AGDVGKSAL (KRAS G13D)
AIDVGKSAY

SEQ ID NO: 445 - protein, 9 aa, synthetic construct
  source 1..9: note = Seed: GAVGVGKSA (KRAS G12V)
GPVGVGKSC

SEQ ID NO: 446 - protein, 9 aa, synthetic construct
  source 1..9: note = Seed: GADGVGKSA (KRAS G12D)
GHDGVGKSL

SEQ ID NO: 447 - protein, 9 aa, synthetic construct
  source 1..9: note = Seed: GAVGVGKSA (KRAS G12V)
GPVGVGKSS

SEQ ID NO: 448 - protein, 9 aa, synthetic construct
  source 1..9: note = Seed: VVGARGVGK (KRAS G12R)
VTGARGVGL

SEQ ID NO: 449 - protein, 9 aa, synthetic construct
  source 1..9: note = Seed: GADGVGKSA (KRAS G12D)
GQDGVGKSF

SEQ ID NO: 450 - protein, 9 aa, synthetic construct
  source 1..9: note = Seed: VVGACGVGK (KRAS G12C)
VLGACGVGR

SEQ ID NO: 451 - protein, 9 aa, synthetic construct
  source 1..9: note = Seed: AGDVGKSAL (KRAS G13D)
AHDVGKSAM

SEQ ID NO: 452 - protein, 9 aa, synthetic construct
  source 1..9: note = Seed: LVVVGACGV (KRAS G12C)
LQVVGACGI

SEQ ID NO: 453 - protein, 9 aa, synthetic construct
  source 1..9: note = Seed: VVGARGVGK (KRAS G12R)
VTGARGVGF

SEQ ID NO: 454 - protein, 9 aa, synthetic construct
  source 1..9: note = Seed: GAGDVGKSA (KRAS G13D)
GAGDVGKSI

SEQ ID NO: 455 - protein, 9 aa, synthetic construct
  source 1..9: note = Seed: VVGADGVGK (KRAS G12D)
VAGADGVGM

SEQ ID NO: 456 - protein, 9 aa, synthetic construct
  source 1..9: note = Seed: GAGDVGKSA (KRAS G13D)
GQGDVGKSY

SEQ ID NO: 457 - protein, 9 aa, synthetic construct
  source 1..9: note = Seed: VVGARGVGK (KRAS G12R)
VAGARGVGK

SEQ ID NO: 458 - protein, 9 aa, synthetic construct
  source 1..9: note = Seed: GAGDVGKSA (KRAS G13D)
GLGDVGKSY

SEQ ID NO: 459 - protein, 9 aa, synthetic construct
  source 1..9: note = Seed: VVGARGVGK (KRAS G12R)
VLGARGVGR

SEQ ID NO: 460 - protein, 9 aa, synthetic construct
  source 1..9: note = Seed: GAVGVGKSA (KRAS G12V)
GGVGVGKSM

SEQ ID NO: 461 - protein, 9 aa, synthetic construct
  source 1..9: note = Seed: GADGVGKSA (KRAS G12D)
GQDGVGKSV

SEQ ID NO: 462 - protein, 9 aa, synthetic construct
  source 1..9: note = Seed: LVVVGAVGV (KRAS G12V)
LQVVGAVGI

SEQ ID NO: 463 - protein, 9 aa, synthetic construct
  source 1..9: note = Seed: LVVVGAVGV (KRAS G12V)
LVVVGAVGI

SEQ ID NO: 464 - protein, 9 aa, synthetic construct
  source 1..9: note = Seed: VVGAVGVGK (KRAS G12V)
VTGAVGVGF

SEQ ID NO: 465 - protein, 9 aa, synthetic construct
  source 1..9: note = Seed: GARGVGKSA (KRAS G12R)
GGRGVGKSF

SEQ ID NO: 466 - protein, 9 aa, synthetic construct
  source 1..9: note = Seed: GARGVGKSA (KRAS G12R)
GLRGVGKSF

SEQ ID NO: 467 - protein, 9 aa, synthetic construct
  source 1..9: note = Seed: GAGDVGKSA (KRAS G13D)
GMGDVGKSY

SEQ ID NO: 468 - protein, 9 aa, synthetic construct
  source 1..9: note = Seed: GADGVGKSA (KRAS G12D)
GQDGVGKSI

SEQ ID NO: 469 - protein, 9 aa, synthetic construct
  source 1..9: note = Seed: VVGAVGVGK (KRAS G12V)
VSGAVGVGM

SEQ ID NO: 470 - protein, 9 aa, synthetic construct
  source 1..9: note = Seed: GAVGVGKSA (KRAS G12V)
GPVGVGKSF

SEQ ID NO: 471 - protein, 9 aa, synthetic construct
  source 1..9: note = Seed: DVGKSALTI (KRAS G13D)
DQGKSALTV

SEQ ID NO: 472 - protein, 9 aa, synthetic construct
  source 1..9: note = Seed: VVGARGVGK (KRAS G12R)
VSGARGVGK

SEQ ID NO: 473 - protein, 9 aa, synthetic construct
  source 1..9: note = Seed: GADGVGKSA (KRAS G12D)
GPDGVGKSL

SEQ ID NO: 474 - protein, 9 aa, synthetic construct
  source 1..9: note = Seed: VVGARGVGK (KRAS G12R)
VAGARGVGR

SEQ ID NO: 475 - protein, 9 aa, synthetic construct
  source 1..9: note = Seed: GADGVGKSA (KRAS G12D)
GPDGVGKSV

SEQ ID NO: 476 - protein, 9 aa, synthetic construct
  source 1..9: note = Seed: GADGVGKSA (KRAS G12D)
GWDGVGKSF

SEQ ID NO: 477 - protein, 9 aa, synthetic construct
  source 1..9: note = Seed: GAVGVGKSA (KRAS G12V)
GVVGVGKSH

SEQ ID NO: 478 - protein, 9 aa, synthetic construct
  source 1..9: note = Seed: VVGARGVGK (KRAS G12R)
VIGARGVGK

SEQ ID NO: 479 - protein, 9 aa, synthetic construct
  source 1..9: note = Seed: DVGKSALTI (KRAS G13D)
DSGKSALTV

SEQ ID NO: 480 - protein, 9 aa, synthetic construct
  source 1..9: note = Seed: DVGKSALTI (KRAS G13D)
DAGKSALTF

SEQ ID NO: 481 - protein, 9 aa, synthetic construct
  source 1..9: note = Seed: GADGVGKSA (KRAS G12D)
GNDGVGKSF

SEQ ID NO: 482 - protein, 9 aa, synthetic construct
  source 1..9: note = Seed: GADGVGKSA (KRAS G12D)
GHDGVGKSM

SEQ ID NO: 483 - protein, 9 aa, synthetic construct
  source 1..9: note = Seed: GACGVGKSA (KRAS G12C)
GICGVGKSM

SEQ ID NO: 484 - protein, 9 aa, synthetic construct
  source 1..9: note = Seed: AGDVGKSAL (KRAS G13D)
ADDVGKSAI

SEQ ID NO: 485 - protein, 9 aa, synthetic construct
  source 1..9: note = Seed: GARGVGKSA (KRAS G12R)
GMRGVGKSF

SEQ ID NO: 486 - protein, 9 aa, synthetic construct
  source 1..9: note = Seed: GARGVGKSA (KRAS G12R)
GPRGVGKSG

SEQ ID NO: 487 - protein, 9 aa, synthetic construct
  source 1..9: note = Seed: LVVVGAVGV (KRAS G12V)
LAVVGAVGV

SEQ ID NO: 488 - protein, 9 aa, synthetic construct
  source 1..9: note = Seed: AGDVGKSAL (KRAS G13D)
ARDVGKSAI

SEQ ID NO: 489 - protein, 9 aa, synthetic construct
  source 1..9: note = Seed: LVVVGACGV (KRAS G12C)
LIVVGACGL

SEQ ID NO: 490 - protein, 9 aa, synthetic construct
  source 1..9: note = Seed: LVVVGAVGV (KRAS G12V)
LLVVGAVGM

SEQ ID NO: 491 - protein, 9 aa, synthetic construct
  source 1..9: note = Seed: GADGVGKSA (KRAS G12D)
GNDGVGKSI

SEQ ID NO: 492 - protein, 9 aa, synthetic construct
  source 1..9: note = Seed: VVGACGVGK (KRAS G12C)
VSGACGVGY

SEQ ID NO: 493 - protein, 9 aa, synthetic construct
  source 1..9: note = Seed: DVGKSALTI (KRAS G13D)
DAGKSALTW

SEQ ID NO: 494 - protein, 9 aa, synthetic construct
  source 1..9: note = Seed: VVGADGVGK (KRAS G12D)
VSGADGVGY

SEQ ID NO: 495 - protein, 9 aa, synthetic construct
  source 1..9: note = Seed: GAVGVGKSA (KRAS G12V)
GTVGVGKSH

SEQ ID NO: 496 - protein, 9 aa, synthetic construct
  source 1..9: note = Seed: GARGVGKSA (KRAS G12R)
GVRGVGKSA

SEQ ID NO: 497 - protein, 9 aa, synthetic construct
  source 1..9: note = Seed: LVVVGAGDV (KRAS G13D)
LMVVGAGDV

SEQ ID NO: 498 - protein, 9 aa, synthetic construct
  source 1..9: note = Seed: VVGACGVGK (KRAS G12C)
VVGACGVGY

SEQ ID NO: 499 - protein, 9 aa, synthetic construct
  source 1..9: note = Seed: DVGKSALTI (KRAS G13D)
DIGKSALTY

SEQ ID NO: 500 - protein, 9 aa, synthetic construct
  source 1..9: note = Seed: GAVGVGKSA (KRAS G12V)
GSVGVGKSV

SEQ ID NO: 501 - protein, 9 aa, synthetic construct
  source 1..9: note = Seed: GADGVGKSA (KRAS G12D)
GNDGVGKSV

SEQ ID NO: 502 - protein, 9 aa, synthetic construct
  source 1..9: note = Seed: GADGVGKSA (KRAS G12D)
GRDGVGKSF

SEQ ID NO: 503 - protein, 9 aa, synthetic construct
  source 1..9: note = Seed: VVGARGVGK (KRAS G12R)
VTGARGVGY

SEQ ID NO: 504 - protein, 9 aa, synthetic construct
  source 1..9: note = Seed: GAGDVGKSA (KRAS G13D)
GIGDVGKSL

SEQ ID NO: 505 - protein, 9 aa, synthetic construct
  source 1..9: note = Seed: LVVVGAVGV (KRAS G12V)
LIVVGAVGI

SEQ ID NO: 506 - protein, 9 aa, synthetic construct
  source 1..9: note = Seed: GADGVGKSA (KRAS G12D)
GIDGVGKSY

SEQ ID NO: 507 - protein, 9 aa, synthetic construct
  source 1..9: note = Seed: GADGVGKSA (KRAS G12D)
GTDGVGKSC

SEQ ID NO: 508 - protein, 9 aa, synthetic construct
  source 1..9: note = Seed: GACGVGKSA (KRAS G12C)
GQCGVGKSY

SEQ ID NO: 509 - protein, 9 aa, synthetic construct
  source 1..9: note = Seed: VVGAVGVGK (KRAS G12V)
VGGAVGVGY

SEQ ID NO: 510 - protein, 9 aa, synthetic construct
  source 1..9: note = Seed: AGDVGKSAL (KRAS G13D)
AHDVGKSAF

SEQ ID NO: 511 - protein, 9 aa, synthetic construct
  source 1..9: note = Seed: GAVGVGKSA (KRAS G12V)
GMVGVGKSF

SEQ ID NO: 512 - protein, 9 aa, synthetic construct
  source 1..9: note = Seed: GACGVGKSA (KRAS G12C)
GMCGVGKSY

SEQ ID NO: 513 - protein, 9 aa, synthetic construct
  source 1..9: note = Seed: AGDVGKSAL (KRAS G13D)
AFDVGKSAW

SEQ ID NO: 514 - protein, 9 aa, synthetic construct
  source 1..9: note = Seed: LVVVGAVGV (KRAS G12V)
LTVVGAVGI

SEQ ID NO: 515 - protein, 9 aa, synthetic construct
  source 1..9: note = Seed: GACGVGKSA (KRAS G12C)
GLCGVGKSY

SEQ ID NO: 516 - protein, 9 aa, synthetic construct
  source 1..9: note = Seed: DVGKSALTI (KRAS G13D)
DGGKSALTV

SEQ ID NO: 517 - protein, 9 aa, synthetic construct
  source 1..9: note = Seed: GARGVGKSA (KRAS G12R)
GNRGVGKSY

SEQ ID NO: 518 - protein, 9 aa, synthetic construct
  source 1..9: note = Seed: VVGAGDVGK (KRAS G13D)
VAGAGDVGR

SEQ ID NO: 519 - protein, 9 aa, synthetic construct
  source 1..9: note = Seed: AGDVGKSAL (KRAS G13D)
APDVGKSAM

SEQ ID NO: 520 - protein, 9 aa, synthetic construct
  source 1..9: note = Seed: AGDVGKSAL (KRAS G13D)
AHDVGKSAY

SEQ ID NO: 521 - protein, 9 aa, synthetic construct
  source 1..9: note = Seed: VVGAGDVGK (KRAS G13D)
VAGAGDVGM

SEQ ID NO: 522 - protein, 9 aa, synthetic construct
  source 1..9: note = Seed: VVGACGVGK (KRAS G12C)
VIGACGVGR

SEQ ID NO: 523 - protein, 9 aa, synthetic construct
  source 1..9: note = Seed: GADGVGKSA (KRAS G12D)
GRDGVGKSI

SEQ ID NO: 524 - protein, 9 aa, synthetic construct
  source 1..9: note = Seed: VVGAVGVGK (KRAS G12V)
VSGAVGVGL

SEQ ID NO: 525 - protein, 9 aa, synthetic construct
  source 1..9: note = Seed: LVVVGAVGV (KRAS G12V)
LVVVGAVGL

SEQ ID NO: 526 - protein, 9 aa, synthetic construct
  source 1..9: note = Seed: VVGARGVGK (KRAS G12R)
VIGARGVGR

SEQ ID NO: 527 - protein, 9 aa, synthetic construct
  source 1..9: note = Seed: GAGDVGKSA (KRAS G13D)
GPGDVGKSL

SEQ ID NO: 528 - protein, 9 aa, synthetic construct
  source 1..9: note = Seed: GARGVGKSA (KRAS G12R)
GSRGVGKSH

SEQ ID NO: 529 - protein, 9 aa, synthetic construct
  source 1..9: note = Seed: AGDVGKSAL (KRAS G13D)
ARDVGKSAV

SEQ ID NO: 530 - protein, 9 aa, synthetic construct
  source 1..9: note = Seed: GAGDVGKSA (KRAS G13D)
GPGDVGKSI

SEQ ID NO: 531 - protein, 9 aa, synthetic construct
  source 1..9: note = Seed: GAGDVGKSA (KRAS G13D)
GPGDVGKSP

SEQ ID NO: 532 - protein, 9 aa, synthetic construct
  source 1..9: note = Seed: AGDVGKSAL (KRAS G13D)
ATDVGKSAC

SEQ ID NO: 533 - protein, 9 aa, synthetic construct
  source 1..9: note = Seed: VVGAGDVGK (KRAS G13D)
VSGAGDVGY

SEQ ID NO: 534 - protein, 9 aa, synthetic construct
  source 1..9: note = Seed: VVGADGVGK (KRAS G12D)
VVGADGVGY

SEQ ID NO: 535 - protein, 9 aa, synthetic construct
  source 1..9: note = Seed: GAVGVGKSA (KRAS G12V)
GQVGVGKSF

SEQ ID NO: 536 - protein, 9 aa, synthetic construct
  source 1..9: note = Seed: VVGADGVGK (KRAS G12D)
VAGADGVGK

SEQ ID NO: 537 - protein, 9 aa, synthetic construct
  source 1..9: note = Seed: DVGKSALTI (KRAS G13D)
DVGKSALTV

SEQ ID NO: 538 - protein, 9 aa, synthetic construct
  source 1..9: note = Seed: GAVGVGKSA (KRAS G12V)
GNVGVGKSY

SEQ ID NO: 539 - protein, 9 aa, synthetic construct
  source 1..9: note = Seed: GAVGVGKSA (KRAS G12V)
GCVGVGKSY

SEQ ID NO: 540 - protein, 9 aa, synthetic construct
  source 1..9: note = Seed: GAVGVGKSA (KRAS G12V)
GLVGVGKSF

SEQ ID NO: 541 - protein, 9 aa, synthetic construct
  source 1..9: note = Seed: GARGVGKSA (KRAS G12R)
GQRGVGKSF

SEQ ID NO: 542 - protein, 9 aa, synthetic construct
  source 1..9: note = Seed: AGDVGKSAL (KRAS G13D)
ADDVGKSAV

SEQ ID NO: 543 - protein, 9 aa, synthetic construct
  source 1..9: note = Seed: LVVVGADGV (KRAS G12D)
LQVVGADGL

SEQ ID NO: 544 - protein, 9 aa, synthetic construct
  source 1..9: note = Seed: GAGDVGKSA (KRAS G13D)
GGGDVGKSF

SEQ ID NO: 545 - protein, 9 aa, synthetic construct
  source 1..9: note = Seed: VVGARGVGK (KRAS G12R)
VAGARGVGV

SEQ ID NO: 546 - protein, 9 aa, synthetic construct
  source 1..9: note = Seed: GARGVGKSA (KRAS G12R)
GSRGVGKSV

SEQ ID NO: 547 - protein, 9 aa, synthetic construct
  source 1..9: note = Seed: GARGVGKSA (KRAS G12R)
GCRGVGKSY

SEQ ID NO: 548 - protein, 9 aa, synthetic construct
  source 1..9: note = Seed: GARGVGKSA (KRAS G12R)
GGRGVGKSM

SEQ ID NO: 549 - protein, 9 aa, synthetic construct
  source 1..9: note = Seed: VVGAVGVGK (KRAS G12V)
VPGAVGVGR

SEQ ID NO: 550 - protein, 9 aa, synthetic construct
  source 1..9: note = Seed: AGDVGKSAL (KRAS G13D)
AADVGKSAW

SEQ ID NO: 551 - protein, 9 aa, synthetic construct
  source 1..9: note = Seed: AGDVGKSAL (KRAS G13D)
ASDVGKSAC

SEQ ID NO: 552 - protein, 9 aa, synthetic construct
  source 1..9: note = Seed: LVVVGACGV (KRAS G12C)
LSVVGACGV

SEQ ID NO: 553 - protein, 9 aa, synthetic construct
  source 1..9: note = Seed: GADGVGKSA (KRAS G12D)
GDDGVGKSL

SEQ ID NO: 554 - protein, 9 aa, synthetic construct
  source 1..9: note = Seed: GADGVGKSA (KRAS G12D)
GSDGVGKSC

SEQ ID NO: 555 - protein, 9 aa, synthetic construct
  source 1..9: note = Seed: GADGVGKSA (KRAS G12D)
GRDGVGKSV

SEQ ID NO: 556 - protein, 9 aa, synthetic construct
  source 1..9: note = Seed: VVGADGVGK (KRAS G12D)
VAGADGVGR

SEQ ID NO: 557 - protein, 9 aa, synthetic construct
  source 1..9: note = Seed: VVGADGVGK (KRAS G12D)
VMGADGVGK

SEQ ID NO: 558 - protein, 9 aa, synthetic construct
  source 1..9: note = Seed: GAGDVGKSA (KRAS G13D)
GQGDVGKSF

SEQ ID NO: 559 - protein, 9 aa, synthetic construct
  source 1..9: note = Seed: AGDVGKSAL (KRAS G13D)
AYDVGKSAW

SEQ ID NO: 560 - protein, 9 aa, synthetic construct
  source 1..9: note = Seed: LVVVGARGV (KRAS G12R)
LQVVGARGV

SEQ ID NO: 561 - protein, 9 aa, synthetic construct
  source 1..9: note = Seed: AGDVGKSAL (KRAS G13D)
ASDVGKSAA

SEQ ID NO: 562 - protein, 9 aa, synthetic construct
  source 1..9: note = Seed: AGDVGKSAL (KRAS G13D)
AGDVGKSAY

SEQ ID NO: 563 - protein, 9 aa, synthetic construct
  source 1..9: note = Seed: VVGAVGVGK (KRAS G12V)
VTGAVGVGM

SEQ ID NO: 564 - protein, 9 aa, synthetic construct
  source 1..9: note = Seed: VVGAVGVGK (KRAS G12V)
VVGAVGVGM

SEQ ID NO: 565 - protein, 9 aa, synthetic construct
  source 1..9: note = Seed: LVVVGADGV (KRAS G12D)
LLVVGADGA

SEQ ID NO: 566 - protein, 9 aa, synthetic construct
  source 1..9: note = Seed: GAVGVGKSA (KRAS G12V)
GAVGVGKSK

SEQ ID NO: 567 - protein, 9 aa, synthetic construct
  source 1..9: note = Seed: GAGDVGKSA (KRAS G13D)
GVGDVGKSH

SEQ ID NO: 568 - protein, 9 aa, synthetic construct
  source 1..9: note = Seed: AGDVGKSAL (KRAS G13D)
AEDVGKSAY

SEQ ID NO: 569 - protein, 9 aa, synthetic construct
  source 1..9: note = Seed: GAGDVGKSA (KRAS G13D)
GPGDVGKST

SEQ ID NO: 570 - protein, 9 aa, synthetic construct
  source 1..9: note = Seed: GAVGVGKSA (KRAS G12V)
GPVGVGKSG

SEQ ID NO: 571 - protein, 9 aa, synthetic construct
  source 1..9: note = Seed: GADGVGKSA (KRAS G12D)
GPDGVGKSM

SEQ ID NO: 572 - protein, 9 aa, synthetic construct
  source 1..9: note = Seed: LVVVGAVGV (KRAS G12V)
LIVVGAVGL

SEQ ID NO: 573 - protein, 9 aa, synthetic construct
  source 1..9: note = Seed: VVGAGDVGK (KRAS G13D)
VMGAGDVGK

SEQ ID NO: 574 - protein, 9 aa, synthetic construct
  source 1..9: note = Seed: GACGVGKSA (KRAS G12C)
GPCGVGKSF

SEQ ID NO: 575 - protein, 9 aa, synthetic construct
  source 1..9: note = Seed: DVGKSALTI (KRAS G13D)
DVGKSALTL

SEQ ID NO: 576 - protein, 9 aa, synthetic construct
  source 1..9: note = Seed: VVGAVGVGK (KRAS G12V)
VTGAVGVGL

SEQ ID NO: 577 - protein, 9 aa, synthetic construct
  source 1..9: note = Seed: AGDVGKSAL (KRAS G13D)
AWDVGKSAC

SEQ ID NO: 578 - protein, 9 aa, synthetic construct
  source 1..9: note = Seed: AGDVGKSAL (KRAS G13D)
AEDVGKSAW

SEQ ID NO: 579 - protein, 9 aa, synthetic construct
  source 1..9: note = Seed: DVGKSALTI (KRAS G13D)
DQGKSALTI

SEQ ID NO: 580 - protein, 9 aa, synthetic construct
  source 1..9: note = Seed: VVGAVGVGK (KRAS G12V)
VYGAVGVGR

SEQ ID NO: 581 - protein, 9 aa, synthetic construct
  source 1..9: note = Seed: GADGVGKSA (KRAS G12D)
GSDGVGKSA

SEQ ID NO: 582 - protein, 9 aa, synthetic construct
  source 1..9: note = Seed: VVGAVGVGK (KRAS G12V)
VGGAVGVGK

SEQ ID NO: 583 - protein, 9 aa, synthetic construct
  source 1..9: note = Seed: GAGDVGKSA (KRAS G13D)
GLGDVGKSF

SEQ ID NO: 584 - protein, 9 aa, synthetic construct
  source 1..9: note = Seed: GAVGVGKSA (KRAS G12V)
GSVGVGKSI

SEQ ID NO: 585 - protein, 9 aa, synthetic construct
  source 1..9: note = Seed: LVVVGACGV (KRAS G12C)
LMVVGACGM

SEQ ID NO: 586 - protein, 9 aa, synthetic construct
  source 1..9: note = Seed: GAVGVGKSA (KRAS G12V)
GAVGVGKSW

SEQ ID NO: 587 - protein, 9 aa, synthetic construct
  source 1..9: note = Seed: GAGDVGKSA (KRAS G13D)
GMGDVGKSF

SEQ ID NO: 588 - protein, 9 aa, synthetic construct
  source 1..9: note = Seed: AGDVGKSAL (KRAS G13D)
AWDVGKSAY

SEQ ID NO: 589 - protein, 9 aa, synthetic construct
  source 1..9: note = Seed: GADGVGKSA (KRAS G12D)
GADGVGKSW

SEQ ID NO: 590 - protein, 9 aa, synthetic construct
  source 1..9: note = Seed: VVGAGDVGK (KRAS G13D)
VAGAGDVGK

SEQ ID NO: 591 - protein, 9 aa, synthetic construct
  source 1..9: note = Seed: LVVVGAVGV (KRAS G12V)
LSVVGAVGV

SEQ ID NO: 592 - protein, 9 aa, synthetic construct
  source 1..9: note = Seed: VVGAVGVGK (KRAS G12V)
VIGAVGVGF

SEQ ID NO: 593 - protein, 9 aa, synthetic construct
  source 1..9: note = Seed: VVGARGVGK (KRAS G12R)
VAGARGVGI

SEQ ID NO: 594 - protein, 9 aa, synthetic construct
  source 1..9: note = Seed: GARGVGKSA (KRAS G12R)
GLRGVGKSM

SEQ ID NO: 595 - protein, 9 aa, synthetic construct
  source 1..9: note = Seed: GAVGVGKSA (KRAS G12V)
GIVGVGKSH

SEQ ID NO: 596 - protein, 9 aa, synthetic construct
  source 1..9: note = Seed: GAVGVGKSA (KRAS G12V)
GMVGVGKSM

SEQ ID NO: 597 - protein, 9 aa, synthetic construct
  source 1..9: note = Seed: GARGVGKSA (KRAS G12R)
GSRGVGKSI

SEQ ID NO: 598 - protein, 9 aa, synthetic construct
  source 1..9: note = Seed: AGDVGKSAL (KRAS G13D)
AADVGKSAT

SEQ ID NO: 599 - protein, 9 aa, synthetic construct
  source 1..9: note = Seed: GACGVGKSA (KRAS G12C)
GGCGVGKSF

SEQ ID NO: 600 - protein, 9 aa, synthetic construct
  source 1..9: note = Seed: GADGVGKSA (KRAS G12D)
GGDGVGKSY

SEQ ID NO: 601 - protein, 9 aa, synthetic construct
  source 1..9: note = Seed: GAVGVGKSA (KRAS G12V)
GAVGVGKSR

SEQ ID NO: 602 - protein, 9 aa, synthetic construct
  source 1..9: note = Seed: GADGVGKSA (KRAS G12D)
GWDGVGKSI

SEQ ID NO: 603 - protein, 9 aa, synthetic construct
  source 1..9: note = Seed: DVGKSALTI (KRAS G13D)
DSGKSALTI

SEQ ID NO: 604 - protein, 9 aa, synthetic construct
  source 1..9: note = Seed: AGDVGKSAL (KRAS G13D)
AMDVGKSAY

SEQ ID NO: 605 - protein, 9 aa, synthetic construct
  source 1..9: note = Seed: GACGVGKSA (KRAS G12C)
GICGVGKSL

SEQ ID NO: 606 - protein, 9 aa, synthetic construct
  source 1..9: note = Seed: GAGDVGKSA (KRAS G13D)
GSGDVGKSH

SEQ ID NO: 607 - protein, 9 aa, synthetic construct
  source 1..9: note = Seed: VVGARGVGK (KRAS G12R)
VSGARGVGR

SEQ ID NO: 608 - protein, 9 aa, synthetic construct
  source 1..9: note = Seed: LVVVGADGV (KRAS G12D)
LVVVGADGL

SEQ ID NO: 609 - protein, 9 aa, synthetic construct
  source 1..9: note = Seed: GADGVGKSA (KRAS G12D)
GHDGVGKSF

SEQ ID NO: 610 - protein, 9 aa, synthetic construct
  source 1..9: note = Seed: DVGKSALTI (KRAS G13D)
DAGKSALTM

SEQ ID NO: 611 - protein, 9 aa, synthetic construct
  source 1..9: note = Seed: AGDVGKSAL (KRAS G13D)
AQDVGKSAY

SEQ ID NO: 612 - protein, 9 aa, synthetic construct
  source 1..9: note = Seed: VVGACGVGK (KRAS G12C)
VSGACGVGF

SEQ ID NO: 613 - protein, 9 aa, synthetic construct
  source 1..9: note = Seed: GAGDVGKSA (KRAS G13D)
GPGDVGKSM

SEQ ID NO: 614 - protein, 9 aa, synthetic construct
  source 1..9: note = Seed: LVVVGAGDV (KRAS G13D)
LLVVGAGDV

SEQ ID NO: 615 - protein, 9 aa, synthetic construct
  source 1..9: note = Seed: AGDVGKSAL (KRAS G13D)
AADVGKSAH

SEQ ID NO: 616 - protein, 9 aa, synthetic construct
  source 1..9: note = Seed: VVGAVGVGK (KRAS G12V)
VGGAVGVGR

SEQ ID NO: 617 - protein, 9 aa, synthetic construct
  source 1..9: note = Seed: GADGVGKSA (KRAS G12D)
GWDGVGKSV

SEQ ID NO: 618 - protein, 9 aa, synthetic construct
  source 1..9: note = Seed: GARGVGKSA (KRAS G12R)
GMRGVGKSM

SEQ ID NO: 619 - protein, 9 aa, synthetic construct
  source 1..9: note = Seed: VVGAGDVGK (KRAS G13D)
VSGAGDVGR

SEQ ID NO: 620 - protein, 9 aa, synthetic construct
  source 1..9: note = Seed: GAVGVGKSA (KRAS G12V)
GQVGVGKSM

SEQ ID NO: 621 - protein, 9 aa, synthetic construct
  source 1..9: note = Seed: VVGADGVGK (KRAS G12D)
VMGADGVGR

SEQ ID NO: 622 - protein, 9 aa, synthetic construct
  source 1..9: note = Seed: GAVGVGKSA (KRAS G12V)
GVVGVGKSK

SEQ ID NO: 623 - protein, 9 aa, synthetic construct
  source 1..9: note = Seed: VVGADGVGK (KRAS G12D)
VIGADGVGK

SEQ ID NO: 624 - protein, 9 aa, synthetic construct
  source 1..9: note = Seed: GAVGVGKSA (KRAS G12V)
GGVGVGKSL

SEQ ID NO: 625 - protein, 9 aa, synthetic construct
  source 1..9: note = Seed: GAGDVGKSA (KRAS G13D)
GNGDVGKSY

SEQ ID NO: 626 - protein, 9 aa, synthetic construct
  source 1..9: note = Seed: GADGVGKSA (KRAS G12D)
GADGVGKST

SEQ ID NO: 627 - protein, 9 aa, synthetic construct
  source 1..9: note = Seed: AGDVGKSAL (KRAS G13D)
AFDVGKSAA

SEQ ID NO: 628 - protein, 9 aa, synthetic construct
  source 1..9: note = Seed: DVGKSALTI (KRAS G13D)
DAGKSALTY

SEQ ID NO: 629 - protein, 9 aa, synthetic construct
  source 1..9: note = Seed: GACGVGKSA (KRAS G12C)
GNCGVGKSY

SEQ ID NO: 630 - protein, 9 aa, synthetic construct
  source 1..9: note = Seed: VVGADGVGK (KRAS G12D)
VSGADGVGK

SEQ ID NO: 631 - protein, 9 aa, synthetic construct
  source 1..9: note = Seed: VVGAGDVGK (KRAS G13D)
VMGAGDVGR

SEQ ID NO: 632 - protein, 9 aa, synthetic construct
  source 1..9: note = Seed: AGDVGKSAL (KRAS G13D)
AKDVGKSAL

SEQ ID NO: 633 - protein, 9 aa, synthetic construct
  source 1..9: note = Seed: AGDVGKSAL (KRAS G13D)
AIDVGKSAC

SEQ ID NO: 634 - protein, 9 aa, synthetic construct
  source 1..9: note = Seed: GACGVGKSA (KRAS G12C)
GSCGVGKSH

SEQ ID NO: 635 - protein, 9 aa, synthetic construct
  source 1..9: note = Seed: GAGDVGKSA (KRAS G13D)
GAGDVGKSK

SEQ ID NO: 636 - protein, 9 aa, synthetic construct
  source 1..9: note = Seed: AGDVGKSAL (KRAS G13D)
AYDVGKSAA

SEQ ID NO: 637 - protein, 9 aa, synthetic construct
  source 1..9: note = Seed: GACGVGKSA (KRAS G12C)
GPCGVGKSC

SEQ ID NO: 638 - protein, 9 aa, synthetic construct
  source 1..9: note = Seed: VVGAVGVGK (KRAS G12V)
VQGAVGVGR

SEQ ID NO: 639 - protein, 9 aa, synthetic construct
  source 1..9: note = Seed: LVVVGARGV (KRAS G12R)
LLVVGARGA

SEQ ID NO: 640 - protein, 9 aa, synthetic construct
  source 1..9: note = Seed: GADGVGKSA (KRAS G12D)
GQDGVGKSY

SEQ ID NO: 641 - protein, 9 aa, synthetic construct
  source 1..9: note = Seed: VVGAGDVGK (KRAS G13D)
VVGAGDVGY

SEQ ID NO: 642 - protein, 9 aa, synthetic construct
  source 1..9: note = Seed: VVGADGVGK (KRAS G12D)
VIGADGVGR

SEQ ID NO: 643 - protein, 9 aa, synthetic construct
  source 1..9: note = Seed: GADGVGKSA (KRAS G12D)
GTDGVGKSA

SEQ ID NO: 644 - protein, 9 aa, synthetic construct
  source 1..9: note = Seed: VVGAVGVGK (KRAS G12V)
VQGAVGVGK

SEQ ID NO: 645 - protein, 9 aa, synthetic construct
  source 1..9: note = Seed: GAGDVGKSA (KRAS G13D)
GVGDVGKSK

SEQ ID NO: 646 - protein, 9 aa, synthetic construct
  source 1..9: note = Seed: GADGVGKSA (KRAS G12D)
GVDGVGKSC

SEQ ID NO: 647 - protein, 9 aa, synthetic construct
  source 1..9: note = Seed: LVVVGACGV (KRAS G12C)
LLVVGACGM

SEQ ID NO: 648 - protein, 9 aa, synthetic construct
  source 1..9: note = Seed: DVGKSALTI (KRAS G13D)
DSGKSALTF

SEQ ID NO: 649 - protein, 9 aa, synthetic construct
  source 1..9: note = Seed: DVGKSALTI (KRAS G13D)
DPGKSALTL

SEQ ID NO: 650 - protein, 9 aa, synthetic construct
  source 1..9: note = Seed: GADGVGKSA (KRAS G12D)
GIDGVGKSC

SEQ ID NO: 651 - protein, 9 aa, synthetic construct
  source 1..9: note = Seed: GAVGVGKSA (KRAS G12V)
GIVGVGKSK

SEQ ID NO: 652 - protein, 9 aa, synthetic construct
  source 1..9: note = Seed: GARGVGKSA (KRAS G12R)
GVRGVGKSH

SEQ ID NO: 653 - protein, 9 aa, synthetic construct
  source 1..9: note = Seed: GADGVGKSA (KRAS G12D)
GMDGVGKSY

SEQ ID NO: 654 - protein, 9 aa, synthetic construct
  source 1..9: note = Seed: GAGDVGKSA (KRAS G13D)
GAGDVGKSR

SEQ ID NO: 655 - protein, 9 aa, synthetic construct
  source 1..9: note = Seed: VVGAGDVGK (KRAS G13D)
VIGAGDVGK

SEQ ID NO: 656 - protein, 9 aa, synthetic construct
  source 1..9: note = Seed: GARGVGKSA (KRAS G12R)
GERGVGKSY

SEQ ID NO: 657 - protein, 9 aa, synthetic construct
  source 1..9: note = Seed: GAGDVGKSA (KRAS G13D)
GVGDVGKSR

SEQ ID NO: 658 - protein, 9 aa, synthetic construct
  source 1..9: note = Seed: GAVGVGKSA (KRAS G12V)
GSVGVGKSK

SEQ ID NO: 659 - protein, 9 aa, synthetic construct
  source 1..9: note = Seed: LVVVGADGV (KRAS G12D)
LVVVGADGI

SEQ ID NO: 660 - protein, 9 aa, synthetic construct
  source 1..9: note = Seed: LVVVGARGV (KRAS G12R)
LIVVGARGV

SEQ ID NO: 661 - protein, 9 aa, synthetic construct
  source 1..9: note = Seed: GAVGVGKSA (KRAS G12V)
GTVGVGKSV

SEQ ID NO: 662 - protein, 9 aa, synthetic construct
  source 1..9: note = Seed: GACGVGKSA (KRAS G12C)
GPCGVGKSS

SEQ ID NO: 663 - protein, 9 aa, synthetic construct
  source 1..9: note = Seed: GAVGVGKSA (KRAS G12V)
GVVGVGKSR

SEQ ID NO: 664 - protein, 9 aa, synthetic construct
  source 1..9: note = Seed: AGDVGKSAL (KRAS G13D)
APDVGKSAV

SEQ ID NO: 665 - protein, 9 aa, synthetic construct
  source 1..9: note = Seed: GADGVGKSA (KRAS G12D)
GVDGVGKSR

SEQ ID NO: 666 - protein, 9 aa, synthetic construct
  source 1..9: note = Seed: GADGVGKSA (KRAS G12D)
GDDGVGKSM

SEQ ID NO: 667 - protein, 9 aa, synthetic construct
  source 1..9: note = Seed: GAGDVGKSA (KRAS G13D)
GGGDVGKSM

SEQ ID NO: 668 - protein, 9 aa, synthetic construct
  source 1..9: note = Seed: AGDVGKSAL (KRAS G13D)
ALDVGKSAY

SEQ ID NO: 669 - protein, 9 aa, synthetic construct
  source 1..9: note = Seed: VVGADGVGK (KRAS G12D)
VSGADGVGR

SEQ ID NO: 670 - protein, 9 aa, synthetic construct
  source 1..9: note = Seed: VVGAVGVGK (KRAS G12V)
VRGAVGVGL

SEQ ID NO: 671 - protein, 9 aa, synthetic construct
  source 1..9: note = Seed: GADGVGKSA (KRAS G12D)
GADGVGKSH

SEQ ID NO: 672 - protein, 9 aa, synthetic construct
  source 1..9: note = Seed: GAVGVGKSA (KRAS G12V)
GTVGVGKSI

SEQ ID NO: 673 - protein, 9 aa, synthetic construct
  source 1..9: note = Seed: VVGACGVGK (KRAS G12C)
VIGACGVGY

SEQ ID NO: 674 - protein, 9 aa, synthetic construct
  source 1..9: note = Seed: VVGAVGVGK (KRAS G12V)
VAGAVGVGI

SEQ ID NO: 675 - protein, 9 aa, synthetic construct
  source 1..9: note = Seed: GARGVGKSA (KRAS G12R)
GVRGVGKSV

SEQ ID NO: 676 - protein, 9 aa, synthetic construct
  source 1..9: note = Seed: AGDVGKSAL (KRAS G13D)
AVDVGKSAC

SEQ ID NO: 677 - protein, 9 aa, synthetic construct
  source 1..9: note = Seed: GACGVGKSA (KRAS G12C)
GMCGVGKSF

SEQ ID NO: 678 - protein, 9 aa, synthetic construct
  source 1..9: note = Seed: GADGVGKSA (KRAS G12D)
GADGVGKSK

SEQ ID NO: 679 - protein, 9 aa, synthetic construct
  source 1..9: note = Seed: AGDVGKSAL (KRAS G13D)
APDVGKSAI

SEQ ID NO: 680 - protein, 9 aa, synthetic construct
  source 1..9: note = Seed: GARGVGKSA (KRAS G12R)
GQRGVGKSM

SEQ ID NO: 681 - protein, 9 aa, synthetic construct
  source 1..9: note = Seed: GADGVGKSA (KRAS G12D)
GVDGVGKSK

SEQ ID NO: 682 - protein, 9 aa, synthetic construct
  source 1..9: note = Seed: GADGVGKSA (KRAS G12D)
GKDGVGKSL

SEQ ID NO: 683 - protein, 9 aa, synthetic construct
  source 1..9: note = Seed: GAVGVGKSA (KRAS G12V)
GLVGVGKSM

SEQ ID NO: 684 - protein, 9 aa, synthetic construct
  source 1..9: note = Seed: GADGVGKSA (KRAS G12D)
GADGVGKSR

SEQ ID NO: 685 - protein, 9 aa, synthetic construct
  source 1..9: note = Seed: VVGAGDVGK (KRAS G13D)
VIGAGDVGR

SEQ ID NO: 686 - protein, 9 aa, synthetic construct
  source 1..9: note = Seed: VVGACGVGK (KRAS G12C)
VVGACGVGF

SEQ ID NO: 687 - protein, 9 aa, synthetic construct
  source 1..9: note = Seed: GARGVGKSA (KRAS G12R)
GVRGVGKSI

SEQ ID NO: 688 - protein, 9 aa, synthetic construct
  source 1..9: note = Seed: GAVGVGKSA (KRAS G12V)
GIVGVGKSR

SEQ ID NO: 689 - protein, 9 aa, synthetic construct
  source 1..9: note = Seed: GAGDVGKSA (KRAS G13D)
GIGDVGKSR

SEQ ID NO: 690 - protein, 9 aa, synthetic construct
  source 1..9: note = Seed: VVGAVGVGK (KRAS G12V)
VAGAVGVGV

SEQ ID NO: 691 - protein, 9 aa, synthetic construct
  source 1..9: note = Seed: GAVGVGKSA (KRAS G12V)
GTVGVGKSK

SEQ ID NO: 692 - protein, 9 aa, synthetic construct
  source 1..9: note = Seed: VVGAVGVGK (KRAS G12V)
VMGAVGVGY

SEQ ID NO: 693 - protein, 9 aa, synthetic construct
  source 1..9: note = Seed: GAGDVGKSA (KRAS G13D)
GIGDVGKSK

SEQ ID NO: 694 - protein, 9 aa, synthetic construct
  source 1..9: note = Seed: LVVVGADGV (KRAS G12D)
LIVVGADGL

SEQ ID NO: 695 - protein, 9 aa, synthetic construct
  source 1..9: note = Seed: GARGVGKSA (KRAS G12R)
GARGVGKSW

SEQ ID NO: 696 - protein, 9 aa, synthetic construct
  source 1..9: note = Seed: LVVVGACGV (KRAS G12C)
LTVVGACGL

SEQ ID NO: 697 - protein, 9 aa, synthetic construct
  source 1..9: note = Seed: GAGDVGKSA (KRAS G13D)
GQGDVGKSM

SEQ ID NO: 698 - protein, 9 aa, synthetic construct
  source 1..9: note = Seed: AGDVGKSAL (KRAS G13D)
ATDVGKSAA

SEQ ID NO: 699 - protein, 9 aa, synthetic construct
  source 1..9: note = Seed: GACGVGKSA (KRAS G12C)
GQCGVGKSF

SEQ ID NO: 700 - protein, 9 aa, synthetic construct
  source 1..9: note = Seed: VVGADGVGK (KRAS G12D)
VSGADGVGF

SEQ ID NO: 701 - protein, 9 aa, synthetic construct
  source 1..9: note = Seed: GADGVGKSA (KRAS G12D)
GPDGVGKSI

SEQ ID NO: 702 - protein, 9 aa, synthetic construct
  source 1..9: note = Seed: GAVGVGKSA (KRAS G12V)
GSVGVGKSR

SEQ ID NO: 703 - protein, 9 aa, synthetic construct
  source 1..9: note = Seed: VVGAGDVGK (KRAS G13D)
VLGAGDVGR

SEQ ID NO: 704 - protein, 9 aa, synthetic construct
  source 1..9: note = Seed: GADGVGKSA (KRAS G12D)
GIDGVGKSR

SEQ ID NO: 705 - protein, 9 aa, synthetic construct
  source 1..9: note = Seed: DVGKSALTI (KRAS G13D)
DGGKSALTI

SEQ ID NO: 706 - protein, 9 aa, synthetic construct
  source 1..9: note = Seed: GARGVGKSA (KRAS G12R)
GTRGVGKSH

SEQ ID NO: 707 - protein, 9 aa, synthetic construct
  source 1..9: note = Seed: GAGDVGKSA (KRAS G13D)
GAGDVGKSW

SEQ ID NO: 708 - protein, 9 aa, synthetic construct
  source 1..9: note = Seed: DVGKSALTI (KRAS G13D)
DSGKSALTW

SEQ ID NO: 709 - protein, 9 aa, synthetic construct
  source 1..9: note = Seed: VVGAGDVGK (KRAS G13D)
VSGAGDVGK

SEQ ID NO: 710 - protein, 9 aa, synthetic construct
  source 1..9: note = Seed: GACGVGKSA (KRAS G12C)
GGCGVGKSM

SEQ ID NO: 711 - protein, 9 aa, synthetic construct
  source 1..9: note = Seed: GARGVGKSA (KRAS G12R)
GMRGVGKSL

SEQ ID NO: 712 - protein, 9 aa, synthetic construct
  source 1..9: note = Seed: GAVGVGKSA (KRAS G12V)
GTVGVGKSR

SEQ ID NO: 713 - protein, 9 aa, synthetic construct
  source 1..9: note = Seed: GAGDVGKSA (KRAS G13D)
GMGDVGKSM

SEQ ID NO: 714 - protein, 9 aa, synthetic construct
  source 1..9: note = Seed: VVGADGVGK (KRAS G12D)
VLGADGVGK

SEQ ID NO: 715 - protein, 9 aa, synthetic construct
  source 1..9: note = Seed: GAVGVGKSA (KRAS G12V)
GEVGVGKSY

SEQ ID NO: 716 - protein, 9 aa, synthetic construct
  source 1..9: note = Seed: VVGACGVGK (KRAS G12C)
VAGACGVGH

SEQ ID NO: 717 - protein, 9 aa, synthetic construct
  source 1..9: note = Seed: LVVVGADGV (KRAS G12D)
LQVVGADGI

SEQ ID NO: 718 - protein, 9 aa, synthetic construct
  source 1..9: note = Seed: VVGAVGVGK (KRAS G12V)
VFGAVGVGR

SEQ ID NO: 719 - protein, 9 aa, synthetic construct
  source 1..9: note = Seed: DVGKSALTI (KRAS G13D)
DQGKSALTL

SEQ ID NO: 720 - protein, 9 aa, synthetic construct
  source 1..9: note = Seed: GAVGVGKSA (KRAS G12V)
GGVGVGKSK

SEQ ID NO: 721 - protein, 9 aa, synthetic construct
  source 1..9: note = Seed: GACGVGKSA (KRAS G12C)
GLCGVGKSF

SEQ ID NO: 722 - protein, 9 aa, synthetic construct
  source 1..9: note = Seed: VVGADGVGK (KRAS G12D)
VLGADGVGR

SEQ ID NO: 723 - protein, 9 aa, synthetic construct
  source 1..9: note = Seed: GAGDVGKSA (KRAS G13D)
GLGDVGKSM

SEQ ID NO: 724 - protein, 9 aa, synthetic construct
  source 1..9: note = Seed: VVGADGVGK (KRAS G12D)
VGGADGVGK

SEQ ID NO: 725 - protein, 9 aa, synthetic construct
  source 1..9: note = Seed: LVVVGARGV (KRAS G12R)
LTVVGARGV

SEQ ID NO: 726 - protein, 9 aa, synthetic construct
  source 1..9: note = Seed: AGDVGKSAL (KRAS G13D)
ALDVGKSAC

SEQ ID NO: 727 - protein, 9 aa, synthetic construct
  source 1..9: note = Seed: GACGVGKSA (KRAS G12C)
GVCGVGKSH

SEQ ID NO: 728 - protein, 9 aa, synthetic construct
  source 1..9: note = Seed: VVGAGDVGK (KRAS G13D)
VLGAGDVGK

SEQ ID NO: 729 - protein, 9 aa, synthetic construct
  source 1..9: note = Seed: LVVVGAVGV (KRAS G12V)
LTVVGAVGL

SEQ ID NO: 730 - protein, 9 aa, synthetic construct
  source 1..9: note = Seed: LVVVGADGV (KRAS G12D)
LIVVGADGI

SEQ ID NO: 731 - protein, 9 aa, synthetic construct
  source 1..9: note = Seed: VVGADGVGK (KRAS G12D)
VGGADGVGR

SEQ ID NO: 732 - protein, 9 aa, synthetic construct
  source 1..9: note = Seed: VVGADGVGK (KRAS G12D)
VAGADGVGH

SEQ ID NO: 733 - protein, 9 aa, synthetic construct
  source 1..9: note = Seed: DVGKSALTI (KRAS G13D)
DLGKSALTW

SEQ ID NO: 734 - protein, 9 aa, synthetic construct
  source 1..9: note = Seed: GADGVGKSA (KRAS G12D)
GEDGVGKSL

SEQ ID NO: 735 - protein, 9 aa, synthetic construct
  source 1..9: note = Seed: GAVGVGKSA (KRAS G12V)
GGVGVGKSR

SEQ ID NO: 736 - protein, 9 aa, synthetic construct
  source 1..9: note = Seed: GADGVGKSA (KRAS G12D)
GLDGVGKSY

SEQ ID NO: 737 - protein, 9 aa, synthetic construct
  source 1..9: note = Seed: GADGVGKSA (KRAS G12D)
GIDGVGKSK

SEQ ID NO: 738 - protein, 9 aa, synthetic construct
  source 1..9: note = Seed: GAGDVGKSA (KRAS G13D)
GTGDVGKSH

SEQ ID NO: 739 - protein, 9 aa, synthetic construct
  source 1..9: note = Seed: VVGADGVGK (KRAS G12D)
VVGADGVGF

SEQ ID NO: 740 - protein, 9 aa, synthetic construct
  source 1..9: note = Seed: LVVVGACGV (KRAS G12C)
LTVVGACGA

SEQ ID NO: 741 - protein, 9 aa, synthetic construct
  source 1..9: note = Seed: VVGADGVGK (KRAS G12D)
VIGADGVGY

SEQ ID NO: 742 - protein, 9 aa, synthetic construct
  source 1..9: note = Seed: GADGVGKSA (KRAS G12D)
GYDGVGKSC

SEQ ID NO: 743 - protein, 9 aa, synthetic construct
  source 1..9: note = Seed: DVGKSALTI (KRAS G13D)
DVGKSALTM

SEQ ID NO: 744 - protein, 9 aa, synthetic construct
  source 1..9: note = Seed: AGDVGKSAL (KRAS G13D)
AHDVGKSAI

SEQ ID NO: 745 - protein, 9 aa, synthetic construct
  source 1..9: note = Seed: GAGDVGKSA (KRAS G13D)
GSGDVGKSK

SEQ ID NO: 746 - protein, 9 aa, synthetic construct
  source 1..9: note = Seed: GAGDVGKSA (KRAS G13D)
GIGDVGKSH

SEQ ID NO: 747 - protein, 9 aa, synthetic construct
  source 1..9: note = Seed: GAGDVGKSA (KRAS G13D)
GTGDVGKSK

SEQ ID NO: 748 - protein, 9 aa, synthetic construct
  source 1..9: note = Seed: GAGDVGKSA (KRAS G13D)
GTGDVGKSR

SEQ ID NO: 749 - protein, 9 aa, synthetic construct
  source 1..9: note = Seed: GADGVGKSA (KRAS G12D)
GTDGVGKSR

SEQ ID NO: 750 - protein, 9 aa, synthetic construct
  source 1..9: note = Seed: GACGVGKSA (KRAS G12C)
GSCGVGKSV

SEQ ID NO: 751 - protein, 9 aa, synthetic construct
  source 1..9: note = Seed: GARGVGKSA (KRAS G12R)
GGRGVGKSL

SEQ ID NO: 752 - protein, 9 aa, synthetic construct
  source 1..9: note = Seed: VVGAGDVGK (KRAS G13D)
VGGAGDVGK

SEQ ID NO: 753 - protein, 9 aa, synthetic construct
  source 1..9: note = Seed: GAVGVGKSA (KRAS G12V)
GVVGVGKSV

SEQ ID NO: 754 - protein, 9 aa, synthetic construct
  source 1..9: note = Seed: GAGDVGKSA (KRAS G13D)
GSGDVGKSR

SEQ ID NO: 755 - protein, 9 aa, synthetic construct
  source 1..9: note = Seed: VVGAVGVGK (KRAS G12V)
VPGAVGVGK

SEQ ID NO: 756 - protein, 9 aa, synthetic construct
  source 1..9: note = Seed: LVVVGACGV (KRAS G12C)
LIVVGACGA

SEQ ID NO: 757 - protein, 9 aa, synthetic construct
  source 1..9: note = Seed: GAGDVGKSA (KRAS G13D)
GCGDVGKSY

SEQ ID NO: 758 - protein, 9 aa, synthetic construct
  source 1..9: note = Seed: AGDVGKSAL (KRAS G13D)
AWDVGKSAW

SEQ ID NO: 759 - protein, 9 aa, synthetic construct
  source 1..9: note = Seed: GADGVGKSA (KRAS G12D)
GTDGVGKSK

SEQ ID NO: 760 - protein, 9 aa, synthetic construct
  source 1..9: note = Seed: VVGACGVGK (KRAS G12C)
VPGACGVGR

SEQ ID NO: 761 - protein, 9 aa, synthetic construct
  source 1..9: note = Seed: GARGVGKSA (KRAS G12R)
GTRGVGKSV

SEQ ID NO: 762 - protein, 9 aa, synthetic construct
  source 1..9: note = Seed: AGDVGKSAL (KRAS G13D)
AIDVGKSAW

SEQ ID NO: 763 - protein, 9 aa, synthetic construct
  source 1..9: note = Seed: GAVGVGKSA (KRAS G12V)
GPVGVGKSY

SEQ ID NO: 764 - protein, 9 aa, synthetic construct
  source 1..9: note = Seed: VVGAVGVGK (KRAS G12V)
VVGAVGVGL

SEQ ID NO: 765 - protein, 9 aa, synthetic construct
  source 1..9: note = Seed: GACGVGKSA (KRAS G12C)
GVCGVGKSK

SEQ ID NO: 766 - protein, 9 aa, synthetic construct
  source 1..9: note = Seed: GAGDVGKSA (KRAS G13D)
GEGDVGKSY

SEQ ID NO: 767 - protein, 9 aa, synthetic construct
  source 1..9: note = Seed: VVGAVGVGK (KRAS G12V)
VQGAVGVGY

SEQ ID NO: 768 - protein, 9 aa, synthetic construct
  source 1..9: note = Seed: VVGAVGVGK (KRAS G12V)
VNGAVGVGK

SEQ ID NO: 769 - protein, 9 aa, synthetic construct
  source 1..9: note = Seed: GACGVGKSA (KRAS G12C)
GACGVGKSW

SEQ ID NO: 770 - protein, 9 aa, synthetic construct
  source 1..9: note = Seed: GADGVGKSA (KRAS G12D)
GGDGVGKSR

SEQ ID NO: 771 - protein, 9 aa, synthetic construct
  source 1..9: note = Seed: GARGVGKSA (KRAS G12R)
GPRGVGKSY

SEQ ID NO: 772 - protein, 9 aa, synthetic construct
  source 1..9: note = Seed: GACGVGKSA (KRAS G12C)
GACGVGKSK

SEQ ID NO: 773 - protein, 9 aa, synthetic construct
  source 1..9: note = Seed: VVGAGDVGK (KRAS G13D)
VSGAGDVGF

SEQ ID NO: 774 - protein, 9 aa, synthetic construct
  source 1..9: note = Seed: GADGVGKSA (KRAS G12D)
GHDGVGKSI

SEQ ID NO: 775 - protein, 9 aa, synthetic construct
  source 1..9: note = Seed: VVGACGVGK (KRAS G12C)
VYGACGVGR

SEQ ID NO: 776 - protein, 9 aa, synthetic construct
  source 1..9: note = Seed: AGDVGKSAL (KRAS G13D)
AMDVGKSAC

SEQ ID NO: 777 - protein, 9 aa, synthetic construct
  source 1..9: note = Seed: GACGVGKSA (KRAS G12C)
GMCGVGKSM

SEQ ID NO: 778 - protein, 9 aa, synthetic construct
  source 1..9: note = Seed: GADGVGKSA (KRAS G12D)
GSDGVGKSR

SEQ ID NO: 779 - protein, 9 aa, synthetic construct
  source 1..9: note = Seed: GARGVGKSA (KRAS G12R)
GPRGVGKSW

SEQ ID NO: 780 - protein, 9 aa, synthetic construct
  source 1..9: note = Seed: GAVGVGKSA (KRAS G12V)
GGVGVGKSH

SEQ ID NO: 781 - protein, 9 aa, synthetic construct
  source 1..9: note = Seed: VVGAVGVGK (KRAS G12V)
VLGAVGVGY

SEQ ID NO: 782 - protein, 9 aa, synthetic construct
  source 1..9: note = Seed: GACGVGKSA (KRAS G12C)
GQCGVGKSM

SEQ ID NO: 783 - protein, 9 aa, synthetic construct
  source 1..9: note = Seed: GAVGVGKSA (KRAS G12V)
GVVGVGKSI

SEQ ID NO: 784 - protein, 9 aa, synthetic construct
  source 1..9: note = Seed: GAGDVGKSA (KRAS G13D)
GVGDVGKSV

SEQ ID NO: 785 - protein, 9 aa, synthetic construct
  source 1..9: note = Seed: GADGVGKSA (KRAS G12D)
GPDGVGKST

SEQ ID NO: 786 - protein, 9 aa, synthetic construct
  source 1..9: note = Seed: AGDVGKSAL (KRAS G13D)
AKDVGKSAM

SEQ ID NO: 787 - protein, 9 aa, synthetic construct
  source 1..9: note = Seed: AGDVGKSAL (KRAS G13D)
APDVGKSAF

SEQ ID NO: 788 - protein, 9 aa, synthetic construct
  source 1..9: note = Seed: VVGAVGVGK (KRAS G12V)
VNGAVGVGR

SEQ ID NO: 789 - protein, 9 aa, synthetic construct
  source 1..9: note = Seed: VVGAGDVGK (KRAS G13D)
VGGAGDVGR

SEQ ID NO: 790 - protein, 9 aa, synthetic construct
  source 1..9: note = Seed: LVVVGACGV (KRAS G12C)
LVVVGACGA

SEQ ID NO: 791 - protein, 9 aa, synthetic construct
  source 1..9: note = Seed: AGDVGKSAL (KRAS G13D)
AEDVGKSAC

SEQ ID NO: 792 - protein, 9 aa, synthetic construct
  source 1..9: note = Seed: GAGDVGKSA (KRAS G13D)
GPGDVGKSC

SEQ ID NO: 793 - protein, 9 aa, synthetic construct
  source 1..9: note = Seed: AGDVGKSAL (KRAS G13D)
AHDVGKSAV

SEQ ID NO: 794 - protein, 9 aa, synthetic construct
  source 1..9: note = Seed: DVGKSALTI (KRAS G13D)
DMGKSALTW

SEQ ID NO: 795 - protein, 9 aa, synthetic construct
  source 1..9: note = Seed: GARGVGKSA (KRAS G12R)
GLRGVGKSL

SEQ ID NO: 796 - protein, 9 aa, synthetic construct
  source 1..9: note = Seed: VVGAVGVGK (KRAS G12V)
VSGAVGVGH

SEQ ID NO: 797 - protein, 9 aa, synthetic construct
  source 1..9: note = Seed: GADGVGKSA (KRAS G12D)
GDDGVGKSF

SEQ ID NO: 798 - protein, 9 aa, synthetic construct
  source 1..9: note = Seed: GADGVGKSA (KRAS G12D)
GGDGVGKSK

SEQ ID NO: 799 - protein, 9 aa, synthetic construct
  source 1..9: note = Seed: GAVGVGKSA (KRAS G12V)
GMVGVGKSL

SEQ ID NO: 800 - protein, 9 aa, synthetic construct
  source 1..9: note = Seed: VVGACGVGK (KRAS G12C)
VGGACGVGK

SEQ ID NO: 801 - protein, 9 aa, synthetic construct
  source 1..9: note = Seed: GADGVGKSA (KRAS G12D)
GSDGVGKSW

SEQ ID NO: 802 - protein, 9 aa, synthetic construct
  source 1..9: note = Seed: GAVGVGKSA (KRAS G12V)
GCVGVGKSF

SEQ ID NO: 803 - protein, 9 aa, synthetic construct
  source 1..9: note = Seed: GAGDVGKSA (KRAS G13D)
GSGDVGKSV

SEQ ID NO: 804 - protein, 9 aa, synthetic construct
  source 1..9: note = Seed: GACGVGKSA (KRAS G12C)
GTCGVGKSH

SEQ ID NO: 805 - protein, 9 aa, synthetic construct
  source 1..9: note = Seed: VVGACGVGK (KRAS G12C)
VFGACGVGR

SEQ ID NO: 806 - protein, 9 aa, synthetic construct
  source 1..9: note = Seed: VVGACGVGK (KRAS G12C)
VQGACGVGK

SEQ ID NO: 807 - protein, 9 aa, synthetic construct
  source 1..9: note = Seed: GAVGVGKSA (KRAS G12V)
GSVGVGKSA

SEQ ID NO: 808 - protein, 9 aa, synthetic construct
  source 1..9: note = Seed: GADGVGKSA (KRAS G12D)
GLDGVGKSR

SEQ ID NO: 809 - protein, 9 aa, synthetic construct
  source 1..9: note = Seed: LVVVGADGV (KRAS G12D)
LAVVGADGV

SEQ ID NO: 810 - protein, 9 aa, synthetic construct
  source 1..9: note = Seed: GACGVGKSA (KRAS G12C)
GECGVGKSY

SEQ ID NO: 811 - protein, 9 aa, synthetic construct
  source 1..9: note = Seed: GADGVGKSA (KRAS G12D)
GSDGVGKSK

SEQ ID NO: 812 - protein, 9 aa, synthetic construct
  source 1..9: note = Seed: GAGDVGKSA (KRAS G13D)
GGGDVGKSR

SEQ ID NO: 813 - protein, 9 aa, synthetic construct
  source 1..9: note = Seed: GACGVGKSA (KRAS G12C)
GPCGVGKSG

SEQ ID NO: 814 - protein, 9 aa, synthetic construct
  source 1..9: note = Seed: AGDVGKSAL (KRAS G13D)
AIDVGKSAA

SEQ ID NO: 815 - protein, 9 aa, synthetic construct
  source 1..9: note = Seed: GACGVGKSA (KRAS G12C)
GACGVGKSR

SEQ ID NO: 816 - protein, 9 aa, synthetic construct
  source 1..9: note = Seed: VVGAVGVGK (KRAS G12V)
VDGAVGVGK

SEQ ID NO: 817 - protein, 9 aa, synthetic construct
  source 1..9: note = Seed: GACGVGKSA (KRAS G12C)
GLCGVGKSM

SEQ ID NO: 818 - protein, 9 aa, synthetic construct
  source 1..9: note = Seed: GACGVGKSA (KRAS G12C)
GSCGVGKSI

SEQ ID NO: 819 - protein, 9 aa, synthetic construct
  source 1..9: note = Seed: GARGVGKSA (KRAS G12R)
GIRGVGKSH

SEQ ID NO: 820 - protein, 9 aa, synthetic construct
  source 1..9: note = Seed: GADGVGKSA (KRAS G12D)
GLDGVGKSC

SEQ ID NO: 821 - protein, 9 aa, synthetic construct
  source 1..9: note = Seed: GAVGVGKSA (KRAS G12V)
GNVGVGKSF

SEQ ID NO: 822 - protein, 9 aa, synthetic construct
  source 1..9: note = Seed: GADGVGKSA (KRAS G12D)
GHDGVGKSV

SEQ ID NO: 823 - protein, 9 aa, synthetic construct
  source 1..9: note = Seed: GACGVGKSA (KRAS G12C)
GICGVGKSK

SEQ ID NO: 824 - protein, 9 aa, synthetic construct
  source 1..9: note = Seed: GARGVGKSA (KRAS G12R)
GTRGVGKSI

SEQ ID NO: 825 - protein, 9 aa, synthetic construct
  source 1..9: note = Seed: GACGVGKSA (KRAS G12C)
GCCGVGKSY

SEQ ID NO: 826 - protein, 9 aa, synthetic construct
  source 1..9: note = Seed: GAVGVGKSA (KRAS G12V)
GLVGVGKSK

SEQ ID NO: 827 - protein, 9 aa, synthetic construct
  source 1..9: note = Seed: VVGARGVGK (KRAS G12R)
VYGARGVGR

SEQ ID NO: 828 - protein, 9 aa, synthetic construct
  source 1..9: note = Seed: DVGKSALTI (KRAS G13D)
DSGKSALTY

SEQ ID NO: 829 - protein, 9 aa, synthetic construct
  source 1..9: note = Seed: VVGACGVGK (KRAS G12C)
VGGACGVGY

SEQ ID NO: 830 - protein, 9 aa, synthetic construct
  source 1..9: note = Seed: GARGVGKSA (KRAS G12R)
GNRGVGKSF

SEQ ID NO: 831 - protein, 9 aa, synthetic construct
  source 1..9: note = Seed: VVGADGVGK (KRAS G12D)
VTGADGVGF

SEQ ID NO: 832 - protein, 9 aa, synthetic construct
  source 1..9: note = Seed: GACGVGKSA (KRAS G12C)
GVCGVGKSV

SEQ ID NO: 833 - protein, 9 aa, synthetic construct
  source 1..9: note = Seed: GAGDVGKSA (KRAS G13D)
GGGDVGKSK

SEQ ID NO: 834 - protein, 9 aa, synthetic construct
  source 1..9: note = Seed: GADGVGKSA (KRAS G12D)
GIDGVGKSW

SEQ ID NO: 835 - protein, 9 aa, synthetic construct
  source 1..9: note = Seed: GADGVGKSA (KRAS G12D)
GVDGVGKSA

SEQ ID NO: 836 - protein, 9 aa, synthetic construct
  source 1..9: note = Seed: AGDVGKSAL (KRAS G13D)
AVDVGKSAA

SEQ ID NO: 837 - protein, 9 aa, synthetic construct
  source 1..9: note = Seed: AGDVGKSAL (KRAS G13D)
ASDVGKSAW

SEQ ID NO: 838 - protein, 9 aa, synthetic construct
  source 1..9: note = Seed: VVGACGVGK (KRAS G12C)
VSGACGVGM

SEQ ID NO: 839 - protein, 9 aa, synthetic construct
  source 1..9: note = Seed: GADGVGKSA (KRAS G12D)
GLDGVGKSK

SEQ ID NO: 840 - protein, 9 aa, synthetic construct
  source 1..9: note = Seed: GACGVGKSA (KRAS G12C)
GVCGVGKSR

SEQ ID NO: 841 - protein, 9 aa, synthetic construct
  source 1..9: note = Seed: GADGVGKSA (KRAS G12D)
GEDGVGKSM

SEQ ID NO: 842 - protein, 9 aa, synthetic construct
  source 1..9: note = Seed: GAGDVGKSA (KRAS G13D)
GTGDVGKSV

SEQ ID NO: 843 - protein, 9 aa, synthetic construct
  source 1..9: note = Seed: VVGACGVGK (KRAS G12C)
VTGACGVGF

SEQ ID NO: 844 - protein, 9 aa, synthetic construct
  source 1..9: note = Seed: GARGVGKSA (KRAS G12R)
GIRGVGKSA

SEQ ID NO: 845 - protein, 9 aa, synthetic construct
  source 1..9: note = Seed: GAGDVGKSA (KRAS G13D)
GLGDVGKSR

SEQ ID NO: 846 - protein, 9 aa, synthetic construct
  source 1..9: note = Seed: VVGAGDVGK (KRAS G13D)
VVGAGDVGF

SEQ ID NO: 847 - protein, 9 aa, synthetic construct
  source 1..9: note = Seed: GAVGVGKSA (KRAS G12V)
GNVGVGKSK

SEQ ID NO: 848 - protein, 9 aa, synthetic construct
  source 1..9: note = Seed: GADGVGKSA (KRAS G12D)
GPDGVGKSP

SEQ ID NO: 849 - protein, 9 aa, synthetic construct
  source 1..9: note = Seed: LVVVGADGV (KRAS G12D)
LMVVGADGM

SEQ ID NO: 850 - protein, 9 aa, synthetic construct
  source 1..9: note = Seed: VVGACGVGK (KRAS G12C)
VQGACGVGR

SEQ ID NO: 851 - protein, 9 aa, synthetic construct
  source 1..9: note = Seed: VVGAVGVGK (KRAS G12V)
VEGAVGVGK

SEQ ID NO: 852 - protein, 9 aa, synthetic construct
  source 1..9: note = Seed: GACGVGKSA (KRAS G12C)
GICGVGKSR

SEQ ID NO: 853 - protein, 9 aa, synthetic construct
  source 1..9: note = Seed: AGDVGKSAL (KRAS G13D)
AVDVGKSAW

SEQ ID NO: 854 - protein, 9 aa, synthetic construct
  source 1..9: note = Seed: GARGVGKSA (KRAS G12R)
GCRGVGKSF

SEQ ID NO: 855 - protein, 9 aa, synthetic construct
  source 1..9: note = Seed: VVGARGVGK (KRAS G12R)
VFGARGVGR

SEQ ID NO: 856 - protein, 9 aa, synthetic construct
  source 1..9: note = Seed: DVGKSALTI (KRAS G13D)
DAGKSALTC

SEQ ID NO: 857 - protein, 9 aa, synthetic construct
  source 1..9: note = Seed: DVGKSALTI (KRAS G13D)
DTGKSALTV

SEQ ID NO: 858 - protein, 9 aa, synthetic construct
  source 1..9: note = Seed: AGDVGKSAL (KRAS G13D)
AEDVGKSAA

SEQ ID NO: 859 - protein, 9 aa, synthetic construct
  source 1..9: note = Seed: GADGVGKSA (KRAS G12D)
GYDGVGKSY

SEQ ID NO: 860 - protein, 9 aa, synthetic construct
  source 1..9: note = Seed: VVGAVGVGK (KRAS G12V)
VGGAVGVGF

SEQ ID NO: 861 - protein, 9 aa, synthetic construct
  source 1..9: note = Seed: VVGARGVGK (KRAS G12R)
VVGARGVGY

SEQ ID NO: 862 - protein, 9 aa, synthetic construct
  source 1..9: note = Seed: AGDVGKSAL (KRAS G13D)
ALDVGKSAW

SEQ ID NO: 863 - protein, 9 aa, synthetic construct
  source 1..9: note = Seed: AGDVGKSAL (KRAS G13D)
AVDVGKSAK

SEQ ID NO: 864 - protein, 9 aa, synthetic construct
  source 1..9: note = Seed: GADGVGKSA (KRAS G12D)
GIDGVGKSA

SEQ ID NO: 865 - protein, 9 aa, synthetic construct
  source 1..9: note = Seed: VVGACGVGK (KRAS G12C)
VGGACGVGR

SEQ ID NO: 866 - protein, 9 aa, synthetic construct
  source 1..9: note = Seed: GAGDVGKSA (KRAS G13D)
GLGDVGKSK

SEQ ID NO: 867 - protein, 9 aa, synthetic construct
  source 1..9: note = Seed: GAVGVGKSA (KRAS G12V)
GLVGVGKSR

SEQ ID NO: 868 - protein, 9 aa, synthetic construct
  source 1..9: note = Seed: VVGAVGVGK (KRAS G12V)
VIGAVGVGM

SEQ ID NO: 869 - protein, 9 aa, synthetic construct
  source 1..9: note = Seed: GAVGVGKSA (KRAS G12V)
GQVGVGKSL

SEQ ID NO: 870 - protein, 9 aa, synthetic construct
  source 1..9: note = Seed: AGDVGKSAL (KRAS G13D)
ATDVGKSAW

SEQ ID NO: 871 - protein, 9 aa, synthetic construct
  source 1..9: note = Seed: DVGKSALTI (KRAS G13D)
DIGKSALTV

SEQ ID NO: 872 - protein, 9 aa, synthetic construct
  source 1..9: note = Seed: VVGARGVGK (KRAS G12R)
VVGARGVGM

SEQ ID NO: 873 - protein, 9 aa, synthetic construct
  source 1..9: note = Seed: GARGVGKSA (KRAS G12R)
GDRGVGKSY

SEQ ID NO: 874 - protein, 9 aa, synthetic construct
  source 1..9: note = Seed: GAVGVGKSA (KRAS G12V)
GDVGVGKSK

SEQ ID NO: 875 - protein, 9 aa, synthetic construct
  source 1..9: note = Seed: GADGVGKSA (KRAS G12D)
GVDGVGKSW

SEQ ID NO: 876 - protein, 9 aa, synthetic construct
  source 1..9: note = Seed: VVGADGVGK (KRAS G12D)
VQGADGVGK

SEQ ID NO: 877 - protein, 9 aa, synthetic construct
  source 1..9: note = Seed: GADGVGKSA (KRAS G12D)
GPDGVGKSF

SEQ ID NO: 878 - protein, 9 aa, synthetic construct
  source 1..9: note = Seed: GAGDVGKSA (KRAS G13D)
GSGDVGKSI

SEQ ID NO: 879 - protein, 9 aa, synthetic construct
  source 1..9: note = Seed: GADGVGKSA (KRAS G12D)
GMDGVGKSR

SEQ ID NO: 880 - protein, 9 aa, synthetic construct
  source 1..9: note = Seed: VVGARGVGK (KRAS G12R)
VVGARGVGF

SEQ ID NO: 881 - protein, 9 aa, synthetic construct
  source 1..9: note = Seed: DVGKSALTI (KRAS G13D)
DMGKSALTV

SEQ ID NO: 882 - protein, 9 aa, synthetic construct
  source 1..9: note = Seed: VVGAGDVGK (KRAS G13D)
VAGAGDVGH

SEQ ID NO: 883 - protein, 9 aa, synthetic construct
  source 1..9: note = Seed: GAVGVGKSA (KRAS G12V)
GAVGVGKSC

SEQ ID NO: 884 - protein, 9 aa, synthetic construct
  source 1..9: note = Seed: VVGADGVGK (KRAS G12D)
VSGADGVGM

SEQ ID NO: 885 - protein, 9 aa, synthetic construct
  source 1..9: note = Seed: AGDVGKSAL (KRAS G13D)
AVDVGKSAR

SEQ ID NO: 886 - protein, 9 aa, synthetic construct
  source 1..9: note = Seed: VVGADGVGK (KRAS G12D)
VQGADGVGR

SEQ ID NO: 887 - protein, 9 aa, synthetic construct
  source 1..9: note = Seed: GARGVGKSA (KRAS G12R)
GSRGVGKSA

SEQ ID NO: 888 - protein, 9 aa, synthetic construct
  source 1..9: note = Seed: AGDVGKSAL (KRAS G13D)
AYDVGKSAT

SEQ ID NO: 889 - protein, 9 aa, synthetic construct
  source 1..9: note = Seed: VVGAVGVGK (KRAS G12V)
VDGAVGVGR

SEQ ID NO: 890 - protein, 9 aa, synthetic construct
  source 1..9: note = Seed: VVGAGDVGK (KRAS G13D)
VIGAGDVGY

SEQ ID NO: 891 - protein, 9 aa, synthetic construct
  source 1..9: note = Seed: GADGVGKSA (KRAS G12D)
GKDGVGKSM

SEQ ID NO: 892 - protein, 9 aa, synthetic construct
  source 1..9: note = Seed: GACGVGKSA (KRAS G12C)
GSCGVGKSK

SEQ ID NO: 893 - protein, 9 aa, synthetic construct
  source 1..9: note = Seed: GAGDVGKSA (KRAS G13D)
GNGDVGKSF

SEQ ID NO: 894 - protein, 9 aa, synthetic construct
  source 1..9: note = Seed: LVVVGACGV (KRAS G12C)
LQVVGACGA

SEQ ID NO: 895 - protein, 9 aa, synthetic construct
  source 1..9: note = Seed: GAGDVGKSA (KRAS G13D)
GVGDVGKSI

SEQ ID NO: 896 - protein, 9 aa, synthetic construct
  source 1..9: note = Seed: VVGADGVGK (KRAS G12D)
VNGADGVGR

SEQ ID NO: 897 - protein, 9 aa, synthetic construct
  source 1..9: note = Seed: AGDVGKSAL (KRAS G13D)
AKDVGKSAF

SEQ ID NO: 898 - protein, 9 aa, synthetic construct
  source 1..9: note = Seed: GAVGVGKSA (KRAS G12V)
GQVGVGKSK

SEQ ID NO: 899 - protein, 9 aa, synthetic construct
  source 1..9: note = Seed: VVGAVGVGK (KRAS G12V)
VRGAVGVGM

SEQ ID NO: 900 - protein, 9 aa, synthetic construct
  source 1..9: note = Seed: AGDVGKSAL (KRAS G13D)
AWDVGKSAA

SEQ ID NO: 901 - protein, 9 aa, synthetic construct
  source 1..9: note = Seed: VVGARGVGK (KRAS G12R)
VVGARGVGL

SEQ ID NO: 902 - protein, 9 aa, synthetic construct
  source 1..9: note = Seed: GAVGVGKSA (KRAS G12V)
GLVGVGKSL

SEQ ID NO: 903 - protein, 9 aa, synthetic construct
  source 1..9: note = Seed: GACGVGKSA (KRAS G12C)
GTCGVGKSK

SEQ ID NO: 904 - protein, 9 aa, synthetic construct
  source 1..9: note = Seed: GADGVGKSA (KRAS G12D)
GSDGVGKST

SEQ ID NO: 905 - protein, 9 aa, synthetic construct
  source 1..9: note = Seed: GARGVGKSA (KRAS G12R)
GQRGVGKSL

SEQ ID NO: 906 - protein, 9 aa, synthetic construct
  source 1..9: note = Seed: DVGKSALTI (KRAS G13D)
DTGKSALTL

SEQ ID NO: 907 - protein, 9 aa, synthetic construct
  source 1..9: note = Seed: GADGVGKSA (KRAS G12D)
GMDGVGKSC

SEQ ID NO: 908 - protein, 9 aa, synthetic construct
  source 1..9: note = Seed: GAVGVGKSA (KRAS G12V)
GAVGVGKST

SEQ ID NO: 909 - protein, 9 aa, synthetic construct
  source 1..9: note = Seed: AGDVGKSAL (KRAS G13D)
AIDVGKSAK

SEQ ID NO: 910 - protein, 9 aa, synthetic construct
  source 1..9: note = Seed: GACGVGKSA (KRAS G12C)
GPCGVGKSY

SEQ ID NO: 911 - protein, 9 aa, synthetic construct
  source 1..9: note = Seed: AGDVGKSAL (KRAS G13D)
AIDVGKSAR

SEQ ID NO: 912 - protein, 9 aa, synthetic construct
  source 1..9: note = Seed: GAGDVGKSA (KRAS G13D)
GQGDVGKSL

SEQ ID NO: 913 - protein, 9 aa, synthetic construct
  source 1..9: note = Seed: VVGARGVGK (KRAS G12R)
VQGARGVGR

SEQ ID NO: 914 - protein, 9 aa, synthetic construct
  source 1..9: note = Seed: GACGVGKSA (KRAS G12C)
GICGVGKSH

SEQ ID NO: 915 - protein, 9 aa, synthetic construct
  source 1..9: note = Seed: GACGVGKSA (KRAS G12C)
GVCGVGKSI

SEQ ID NO: 916 - protein, 9 aa, synthetic construct
  source 1..9: note = Seed: GADGVGKSA (KRAS G12D)
GMDGVGKSK

SEQ ID NO: 917 - protein, 9 aa, synthetic construct
  source 1..9: note = Seed: GAGDVGKSA (KRAS G13D)
GFGDVGKSY

SEQ ID NO: 918 - protein, 9 aa, synthetic construct
  source 1..9: note = Seed: GADGVGKSA (KRAS G12D)
GTDGVGKSW

SEQ ID NO: 919 - protein, 9 aa, synthetic construct
  source 1..9: note = Seed: LVVVGADGV (KRAS G12D)
LLVVGADGM

SEQ ID NO: 920 - protein, 9 aa, synthetic construct
  source 1..9: note = Seed: AGDVGKSAL (KRAS G13D)
AADVGKSAK

SEQ ID NO: 921 - protein, 9 aa, synthetic construct
  source 1..9: note = Seed: GAGDVGKSA (KRAS G13D)
GTGDVGKSI

SEQ ID NO: 922 - protein, 9 aa, synthetic construct
  source 1..9: note = Seed: VVGAVGVGK (KRAS G12V)
VFGAVGVGK

SEQ ID NO: 923 - protein, 9 aa, synthetic construct
  source 1..9: note = Seed: GACGVGKSA (KRAS G12C)
GSCGVGKSR

SEQ ID NO: 924 - protein, 9 aa, synthetic construct
  source 1..9: note = Seed: GACGVGKSA (KRAS G12C)
GTCGVGKSV

SEQ ID NO: 925 - protein, 9 aa, synthetic construct
  source 1..9: note = Seed: DVGKSALTI (KRAS G13D)
DAGKSALTA

SEQ ID NO: 926 - protein, 9 aa, synthetic construct
  source 1..9: note = Seed: GACGVGKSA (KRAS G12C)
GTCGVGKSR

SEQ ID NO: 927 - protein, 9 aa, synthetic construct
  source 1..9: note = Seed: VVGAVGVGK (KRAS G12V)
VYGAVGVGK

SEQ ID NO: 928 - protein, 9 aa, synthetic construct
  source 1..9: note = Seed: GARGVGKSA (KRAS G12R)
GIRGVGKSV

SEQ ID NO: 929 - protein, 9 aa, synthetic construct
  source 1..9: note = Seed: VVGARGVGK (KRAS G12R)
VSGARGVGV

SEQ ID NO: 930 - protein, 9 aa, synthetic construct
  source 1..9: note = Seed: VVGAGDVGK (KRAS G13D)
VNGAGDVGK

SEQ ID NO: 931 - protein, 9 aa, synthetic construct
  source 1..9: note = Seed: VVGAGDVGK (KRAS G13D)
VQGAGDVGK

SEQ ID NO: 932 - protein, 9 aa, synthetic construct
  source 1..9: note = Seed: GADGVGKSA (KRAS G12D)
GQDGVGKSK

SEQ ID NO: 933 - protein, 9 aa, synthetic construct
  source 1..9: note = Seed: GAVGVGKSA (KRAS G12V)
GNVGVGKSR

SEQ ID NO: 934 - protein, 9 aa, synthetic construct
  source 1..9: note = Seed: GADGVGKSA (KRAS G12D)
GNDGVGKSR

SEQ ID NO: 935 - protein, 9 aa, synthetic construct
  source 1..9: note = Seed: VVGAVGVGK (KRAS G12V)
VAGAVGVGA

SEQ ID NO: 936 - protein, 9 aa, synthetic construct
  source 1..9: note = Seed: VVGADGVGK (KRAS G12D)
VNGADGVGK

SEQ ID NO: 937 - protein, 9 aa, synthetic construct
  source 1..9: note = Seed: GAGDVGKSA (KRAS G13D)
GPGDVGKSY

SEQ ID NO: 938 - protein, 9 aa, synthetic construct
  source 1..9: note = Seed: GACGVGKSA (KRAS G12C)
GGCGVGKSL

SEQ ID NO: 939 - protein, 9 aa, synthetic construct
  source 1..9: note = Seed: VVGAGDVGK (KRAS G13D)
VQGAGDVGR

SEQ ID NO: 940 - protein, 9 aa, synthetic construct
  source 1..9: note = Seed: GACGVGKSA (KRAS G12C)
GGCGVGKSR

SEQ ID NO: 941 - protein, 9 aa, synthetic construct
  source 1..9: note = Seed: GADGVGKSA (KRAS G12D)
GQDGVGKSR

SEQ ID NO: 942 - protein, 9 aa, synthetic construct
  source 1..9: note = Seed: GAGDVGKSA (KRAS G13D)
GGGDVGKSL

SEQ ID NO: 943 - protein, 9 aa, synthetic construct
  source 1..9: note = Seed: VVGAGDVGK (KRAS G13D)
VTGAGDVGF

SEQ ID NO: 944 - protein, 9 aa, synthetic construct
  source 1..9: note = Seed: VVGARGVGK (KRAS G12R)
VSGARGVGI

SEQ ID NO: 945 - protein, 9 aa, synthetic construct
  source 1..9: note = Seed: GARGVGKSA (KRAS G12R)
GARGVGKSC

SEQ ID NO: 946 - protein, 9 aa, synthetic construct
  source 1..9: note = Seed: AGDVGKSAL (KRAS G13D)
ASDVGKSAT

SEQ ID NO: 947 - protein, 9 aa, synthetic construct
  source 1..9: note = Seed: VVGAVGVGK (KRAS G12V)
VVGAVGVGH

SEQ ID NO: 948 - protein, 9 aa, synthetic construct
  source 1..9: note = Seed: GAVGVGKSA (KRAS G12V)
GDVGVGKSR

SEQ ID NO: 949 - protein, 9 aa, synthetic construct
  source 1..9: note = Seed: AGDVGKSAL (KRAS G13D)
AFDVGKSAT

SEQ ID NO: 950 - protein, 9 aa, synthetic construct
  source 1..9: note = Seed: VVGACGVGK (KRAS G12C)
VVGACGVGM

SEQ ID NO: 951 - protein, 9 aa, synthetic construct
  source 1..9: note = Seed: GARGVGKSA (KRAS G12R)
GIRGVGKSI

SEQ ID NO: 952 - protein, 9 aa, synthetic construct
  source 1..9: note = Seed: VVGAGDVGK (KRAS G13D)
VNGAGDVGR

SEQ ID NO: 953 - protein, 9 aa, synthetic construct
  source 1..9: note = Seed: GACGVGKSA (KRAS G12C)
GVCGVGKSA

SEQ ID NO: 954 - protein, 9 aa, synthetic construct
  source 1..9: note = Seed: VVGACGVGK (KRAS G12C)
VIGACGVGF

SEQ ID NO: 955 - protein, 9 aa, synthetic construct
  source 1..9: note = Seed: GACGVGKSA (KRAS G12C)
GGCGVGKSK

SEQ ID NO: 956 - protein, 9 aa, synthetic construct
  source 1..9: note = Seed: GARGVGKSA (KRAS G12R)
GFRGVGKSY

SEQ ID NO: 957 - protein, 9 aa, synthetic construct
  source 1..9: note = Seed: VVGAVGVGK (KRAS G12V)
VMGAVGVGF

SEQ ID NO: 958 - protein, 9 aa, synthetic construct
  source 1..9: note = Seed: VVGAGDVGK (KRAS G13D)
VPGAGDVGR

SEQ ID NO: 959 - protein, 9 aa, synthetic construct
  source 1..9: note = Seed: GARGVGKSA (KRAS G12R)
GPRGVGKSQ

SEQ ID NO: 960 - protein, 9 aa, synthetic construct
  source 1..9: note = Seed: GACGVGKSA (KRAS G12C)
GTCGVGKSI

SEQ ID NO: 961 - protein, 9 aa, synthetic construct
  source 1..9: note = Seed: GADGVGKSA (KRAS G12D)
GNDGVGKSK

SEQ ID NO: 962 - protein, 9 aa, synthetic construct
  source 1..9: note = Seed: GAGDVGKSA (KRAS G13D)
GMGDVGKSL

SEQ ID NO: 963 - protein, 9 aa, synthetic construct
  source 1..9: note = Seed: GAGDVGKSA (KRAS G13D)
GLGDVGKSL

SEQ ID NO: 964 - protein, 9 aa, synthetic construct
  source 1..9: note = Seed: GAVGVGKSA (KRAS G12V)
GCVGVGKSM

SEQ ID NO: 965 - protein, 9 aa, synthetic construct
  source 1..9: note = Seed: VVGAVGVGK (KRAS G12V)
VEGAVGVGR

SEQ ID NO: 966 - protein, 9 aa, synthetic construct
  source 1..9: note = Seed: GAVGVGKSA (KRAS G12V)
GDVGVGKSY

SEQ ID NO: 967 - protein, 9 aa, synthetic construct
  source 1..9: note = Seed: GACGVGKSA (KRAS G12C)
GNCGVGKSF

SEQ ID NO: 968 - protein, 9 aa, synthetic construct
  source 1..9: note = Seed: AGDVGKSAL (KRAS G13D)
AADVGKSAR

SEQ ID NO: 969 - protein, 9 aa, synthetic construct
  source 1..9: note = Seed: GADGVGKSA (KRAS G12D)
GNDGVGKSY

SEQ ID NO: 970 - protein, 9 aa, synthetic construct
  source 1..9: note = Seed: LVVVGAVGV (KRAS G12V)
LVVVGAVGA

SEQ ID NO: 971 - protein, 9 aa, synthetic construct
  source 1..9: note = Seed: DVGKSALTI (KRAS G13D)
DSGKSALTL

SEQ ID NO: 972 - protein, 9 aa, synthetic construct
  source 1..9: note = Seed: VVGARGVGK (KRAS G12R)
VPGARGVGR

SEQ ID NO: 973 - protein, 9 aa, synthetic construct
  source 1..9: note = Seed: VVGACGVGK (KRAS G12C)
VMGACGVGY

SEQ ID NO: 974 - protein, 9 aa, synthetic construct
  source 1..9: note = Seed: AGDVGKSAL (KRAS G13D)
ADDVGKSAY

SEQ ID NO: 975 - protein, 9 aa, synthetic construct
  source 1..9: note = Seed: VVGAGDVGK (KRAS G13D)
VFGAGDVGK

SEQ ID NO: 976 - protein, 9 aa, synthetic construct
  source 1..9: note = Seed: GAVGVGKSA (KRAS G12V)
GMVGVGKSK

SEQ ID NO: 977 - protein, 9 aa, synthetic construct
  source 1..9: note = Seed: GAVGVGKSA (KRAS G12V)
GVVGVGKSA

SEQ ID NO: 978 - protein, 9 aa, synthetic construct
  source 1..9: note = Seed: GADGVGKSA (KRAS G12D)
GFDGVGKSC

SEQ ID NO: 979 - protein, 9 aa, synthetic construct
  source 1..9: note = Seed: GAGDVGKSA (KRAS G13D)
GPGDVGKSS

SEQ ID NO: 980 - protein, 9 aa, synthetic construct
  source 1..9: note = Seed: GAVGVGKSA (KRAS G12V)
GSVGVGKSW

SEQ ID NO: 981 - protein, 9 aa, synthetic construct
  source 1..9: note = Seed: GAGDVGKSA (KRAS G13D)
GDGDVGKSK

SEQ ID NO: 982 - protein, 9 aa, synthetic construct
  source 1..9: note = Seed: LVVVGADGV (KRAS G12D)
LTVVGADGL

SEQ ID NO: 983 - protein, 9 aa, synthetic construct
  source 1..9: note = Seed: DVGKSALTI (KRAS G13D)
DIGKSALTI

SEQ ID NO: 984 - protein, 9 aa, synthetic construct
  source 1..9: note = Seed: GADGVGKSA (KRAS G12D)
GFDGVGKSY

SEQ ID NO: 985 - protein, 9 aa, synthetic construct
  source 1..9: note = Seed: AGDVGKSAL (KRAS G13D)
ADDVGKSAW

SEQ ID NO: 986 - protein, 9 aa, synthetic construct
  source 1..9: note = Seed: LVVVGAVGV (KRAS G12V)
LIVVGAVGA

SEQ ID NO: 987 - protein, 9 aa, synthetic construct
  source 1..9: note = Seed: LVVVGAVGV (KRAS G12V)
LTVVGAVGA

SEQ ID NO: 988 - protein, 9 aa, synthetic construct
  source 1..9: note = Seed: GARGVGKSA (KRAS G12R)
GERGVGKSF

SEQ ID NO: 989 - protein, 9 aa, synthetic construct
  source 1..9: note = Seed: DVGKSALTI (KRAS G13D)
DTGKSALTI

SEQ ID NO: 990 - protein, 9 aa, synthetic construct
  source 1..9: note = Seed: GAVGVGKSA (KRAS G12V)
GQVGVGKSR

SEQ ID NO: 991 - protein, 9 aa, synthetic construct
  source 1..9: note = Seed: GAGDVGKSA (KRAS G13D)
GNGDVGKSK

SEQ ID NO: 992 - protein, 9 aa, synthetic construct
  source 1..9: note = Seed: GAGDVGKSA (KRAS G13D)
GNGDVGKSR

SEQ ID NO: 993 - protein, 9 aa, synthetic construct
  source 1..9: note = Seed: VVGADGVGK (KRAS G12D)
VMGADGVGY

SEQ ID NO: 994 - protein, 9 aa, synthetic construct
  source 1..9: note = Seed: GAGDVGKSA (KRAS G13D)
GMGDVGKSR

SEQ ID NO: 995 - protein, 9 aa, synthetic construct
  source 1..9: note = Seed: VVGADGVGK (KRAS G12D)
VDGADGVGK

SEQ ID NO: 996 - protein, 9 aa, synthetic construct
  source 1..9: note = Seed: GACGVGKSA (KRAS G12C)
GMCGVGKSL

SEQ ID NO: 997 - protein, 9 aa, synthetic construct
  source 1..9: note = Seed: VVGADGVGK (KRAS G12D)
VPGADGVGR

SEQ ID NO: 998 - protein, 9 aa, synthetic construct
  source 1..9: note = Seed: AGDVGKSAL (KRAS G13D)
ANDVGKSAY

SEQ ID NO: 999 - protein, 9 aa, synthetic construct
  source 1..9: note = Seed: AGDVGKSAL (KRAS G13D)
AGDVGKSAK

SEQ ID NO: 1000 - protein, 9 aa, synthetic construct
  source 1..9: note = Seed: VVGADGVGK (KRAS G12D)
VPGADGVGK

SEQ ID NO: 1001 - protein, 9 aa, synthetic construct
  source 1..9: note = Seed: GAVGVGKSA (KRAS G12V)
GMVGVGKSH

SEQ ID NO: 1002 - protein, 9 aa, synthetic construct
  source 1..9: note = Seed: GADGVGKSA (KRAS G12D)
GPDGVGKSC

SEQ ID NO: 1003 - protein, 9 aa, synthetic construct
  source 1..9: note = Seed: VVGACGVGK (KRAS G12C)
VSGACGVGL

SEQ ID NO: 1004 - protein, 9 aa, synthetic construct
  source 1..9: note = Seed: GAGDVGKSA (KRAS G13D)
GEGDVGKSF

SEQ ID NO: 1005 - protein, 9 aa, synthetic construct
  source 1..9: note = Seed: VVGACGVGK (KRAS G12C)
VLGACGVGY

SEQ ID NO: 1006 - protein, 9 aa, synthetic construct
  source 1..9: note = Seed: VVGADGVGK (KRAS G12D)
VGGADGVGY

SEQ ID NO: 1007 - protein, 9 aa, synthetic construct
  source 1..9: note = Seed: GAVGVGKSA (KRAS G12V)
GMVGVGKSR

SEQ ID NO: 1008 - protein, 9 aa, synthetic construct
  source 1..9: note = Seed: VVGAVGVGK (KRAS G12V)
VWGAVGVGR

SEQ ID NO: 1009 - protein, 9 aa, synthetic construct
  source 1..9: note = Seed: GADGVGKSA (KRAS G12D)
GTDGVGKST

SEQ ID NO: 1010 - protein, 9 aa, synthetic construct
  source 1..9: note = Seed: VVGADGVGK (KRAS G12D)
VLGADGVGY

SEQ ID NO: 1011 - protein, 9 aa, synthetic construct
  source 1..9: note = Seed: VVGAGDVGK (KRAS G13D)
VSGAGDVGM

SEQ ID NO: 1012 - protein, 9 aa, synthetic construct
  source 1..9: note = Seed: GAGDVGKSA (KRAS G13D)
GMGDVGKSK

SEQ ID NO: 1013 - protein, 9 aa, synthetic construct
  source 1..9: note = Seed: GAGDVGKSA (KRAS G13D)
GDGDVGKSR

SEQ ID NO: 1014 - protein, 9 aa, synthetic construct
  source 1..9: note = Seed: VVGACGVGK (KRAS G12C)
VPGACGVGK

SEQ ID NO: 1015 - protein, 9 aa, synthetic construct
  source 1..9: note = Seed: GADGVGKSA (KRAS G12D)
GYDGVGKSA

SEQ ID NO: 1016 - protein, 9 aa, synthetic construct
  source 1..9: note = Seed: VVGADGVGK (KRAS G12D)
VFGADGVGK

SEQ ID NO: 1017 - protein, 9 aa, synthetic construct
  source 1..9: note = Seed: VVGAVGVGK (KRAS G12V)
VQGAVGVGF

SEQ ID NO: 1018 - protein, 9 aa, synthetic construct
  source 1..9: note = Seed: GAGDVGKSA (KRAS G13D)
GCGDVGKSF

SEQ ID NO: 1019 - protein, 9 aa, synthetic construct
  source 1..9: note = Seed: AGDVGKSAL (KRAS G13D)
ARDVGKSAW

SEQ ID NO: 1020 - protein, 9 aa, synthetic construct
  source 1..9: note = Seed: GARGVGKSA (KRAS G12R)
GGRGVGKSA

SEQ ID NO: 1021 - protein, 9 aa, synthetic construct
  source 1..9: note = Seed: AGDVGKSAL (KRAS G13D)
AKDVGKSAY

SEQ ID NO: 1022 - protein, 9 aa, synthetic construct
  source 1..9: note = Seed: VVGAVGVGK (KRAS G12V)
VRGAVGVGI

SEQ ID NO: 1023 - protein, 9 aa, synthetic construct
  source 1..9: note = Seed: VVGADGVGK (KRAS G12D)
VEGADGVGK

SEQ ID NO: 1024 - protein, 9 aa, synthetic construct
  source 1..9: note = Seed: GACGVGKSA (KRAS G12C)
GLCGVGKSR

SEQ ID NO: 1025 - protein, 9 aa, synthetic construct
  source 1..9: note = Seed: GADGVGKSA (KRAS G12D)
GDDGVGKSI

SEQ ID NO: 1026 - protein, 9 aa, synthetic construct
  source 1..9: note = Seed: GAVGVGKSA (KRAS G12V)
GFVGVGKSY

SEQ ID NO: 1027 - protein, 9 aa, synthetic construct
  source 1..9: note = Seed: DVGKSALTI (KRAS G13D)
DTGKSALTM

SEQ ID NO: 1028 - protein, 9 aa, synthetic construct
  source 1..9: note = Seed: VVGARGVGK (KRAS G12R)
VIGARGVGY

SEQ ID NO: 1029 - protein, 9 aa, synthetic construct
  source 1..9: note = Seed: VVGAVGVGK (KRAS G12V)
VTGAVGVGH

SEQ ID NO: 1030 - protein, 9 aa, synthetic construct
  source 1..9: note = Seed: AGDVGKSAL (KRAS G13D)
AGDVGKSAR

SEQ ID NO: 1031 - protein, 9 aa, synthetic construct
  source 1..9: note = Seed: GACGVGKSA (KRAS G12C)
GQCGVGKSL

SEQ ID NO: 1032 - protein, 9 aa, synthetic construct
  source 1..9: note = Seed: GAVGVGKSA (KRAS G12V)
GNVGVGKSM

SEQ ID NO: 1033 - protein, 9 aa, synthetic construct
  source 1..9: note = Seed: VVGAGDVGK (KRAS G13D)
VMGAGDVGY

SEQ ID NO: 1034 - protein, 9 aa, synthetic construct
  source 1..9: note = Seed: GADGVGKSA (KRAS G12D)
GFDGVGKSK

SEQ ID NO: 1035 - protein, 9 aa, synthetic construct
  source 1..9: note = Seed: VVGAGDVGK (KRAS G13D)
VDGAGDVGK

SEQ ID NO: 1036 - protein, 9 aa, synthetic construct
  source 1..9: note = Seed: AGDVGKSAL (KRAS G13D)
AQDVGKSAW

SEQ ID NO: 1037 - protein, 9 aa, synthetic construct
  source 1..9: note = Seed: GADGVGKSA (KRAS G12D)
GDDGVGKSK

SEQ ID NO: 1038 - protein, 9 aa, synthetic construct
  source 1..9: note = Seed: VVGARGVGK (KRAS G12R)
VTGARGVGI

SEQ ID NO: 1039 - protein, 9 aa, synthetic construct
  source 1..9: note = Seed: LVVVGADGV (KRAS G12D)
LSVVGADGV

SEQ ID NO: 1040 - protein, 9 aa, synthetic construct
  source 1..9: note = Seed: VVGADGVGK (KRAS G12D)
VVGADGVGM

SEQ ID NO: 1041 - protein, 9 aa, synthetic construct
  source 1..9: note = Seed: GARGVGKSA (KRAS G12R)
GNRGVGKSM

SEQ ID NO: 1042 - protein, 9 aa, synthetic construct
  source 1..9: note = Seed: VVGAVGVGK (KRAS G12V)
VGGAVGVGM

SEQ ID NO: 1043 - protein, 9 aa, synthetic construct
  source 1..9: note = Seed: GAVGVGKSA (KRAS G12V)
GEVGVGKSK

SEQ ID NO: 1044 - protein, 9 aa, synthetic construct
  source 1..9: note = Seed: LVVVGAVGV (KRAS G12V)
LQVVGAVGA

SEQ ID NO: 1045 - protein, 9 aa, synthetic construct
  source 1..9: note = Seed: GARGVGKSA (KRAS G12R)
GPRGVGKSH

SEQ ID NO: 1046 - protein, 9 aa, synthetic construct
  source 1..9: note = Seed: GADGVGKSA (KRAS G12D)
GLDGVGKSW

SEQ ID NO: 1047 - protein, 9 aa, synthetic construct
  source 1..9: note = Seed: VVGARGVGK (KRAS G12R)
VTGARGVGV

SEQ ID NO: 1048 - protein, 9 aa, synthetic construct
  source 1..9: note = Seed: GAGDVGKSA (KRAS G13D)
GQGDVGKSR

SEQ ID NO: 1049 - protein, 9 aa, synthetic construct
  source 1..9: note = Seed: GADGVGKSA (KRAS G12D)
GEDGVGKSF

SEQ ID NO: 1050 - protein, 9 aa, synthetic construct
  source 1..9: note = Seed: VVGADGVGK (KRAS G12D)
VIGADGVGF

SEQ ID NO: 1051 - protein, 9 aa, synthetic construct
  source 1..9: note = Seed: GAGDVGKSA (KRAS G13D)
GQGDVGKSK

SEQ ID NO: 1052 - protein, 9 aa, synthetic construct
  source 1..9: note = Seed: VVGARGVGK (KRAS G12R)
VGGARGVGY

SEQ ID NO: 1053 - protein, 9 aa, synthetic construct
  source 1..9: note = Seed: VVGARGVGK (KRAS G12R)
VQGARGVGK

SEQ ID NO: 1054 - protein, 9 aa, synthetic construct
  source 1..9: note = Seed: GADGVGKSA (KRAS G12D)
GKDGVGKSF

SEQ ID NO: 1055 - protein, 9 aa, synthetic construct
  source 1..9: note = Seed: VVGAVGVGK (KRAS G12V)
VRGAVGVGV

SEQ ID NO: 1056 - protein, 9 aa, synthetic construct
  source 1..9: note = Seed: VVGACGVGK (KRAS G12C)
VDGACGVGK

SEQ ID NO: 1057 - protein, 9 aa, synthetic construct
  source 1..9: note = Seed: AGDVGKSAL (KRAS G13D)
ASDVGKSAK

SEQ ID NO: 1058 - protein, 9 aa, synthetic construct
  source 1..9: note = Seed: GAVGVGKSA (KRAS G12V)
GQVGVGKSH

SEQ ID NO: 1059 - protein, 9 aa, synthetic construct
  source 1..9: note = Seed: AGDVGKSAL (KRAS G13D)
ASDVGKSAR

SEQ ID NO: 1060 - protein, 9 aa, synthetic construct
  source 1..9: note = Seed: AGDVGKSAL (KRAS G13D)
ASDVGKSAH

SEQ ID NO: 1061 - protein, 9 aa, synthetic construct
  source 1..9: note = Seed: GAVGVGKSA (KRAS G12V)
GEVGVGKSF

SEQ ID NO: 1062 - protein, 9 aa, synthetic construct
  source 1..9: note = Seed: VVGAGDVGK (KRAS G13D)
VLGAGDVGY

SEQ ID NO: 1063 - protein, 9 aa, synthetic construct
  source 1..9: note = Seed: GARGVGKSA (KRAS G12R)
GARGVGKST

SEQ ID NO: 1064 - protein, 9 aa, synthetic construct
  source 1..9: note = Seed: DVGKSALTI (KRAS G13D)
DMGKSALTI

SEQ ID NO: 1065 - protein, 9 aa, synthetic construct
  source 1..9: note = Seed: LVVVGADGV (KRAS G12D)
LTVVGADGI

SEQ ID NO: 1066 - protein, 9 aa, synthetic construct
  source 1..9: note = Seed: GAVGVGKSA (KRAS G12V)
GLVGVGKSH

SEQ ID NO: 1067 - protein, 9 aa, synthetic construct
  source 1..9: note = Seed: GACGVGKSA (KRAS G12C)
GLCGVGKSL

SEQ ID NO: 1068 - protein, 9 aa, synthetic construct
  source 1..9: note = Seed: VVGARGVGK (KRAS G12R)
VIGARGVGF

SEQ ID NO: 1069 - protein, 9 aa, synthetic construct
  source 1..9: note = Seed: GADGVGKSA (KRAS G12D)
GDDGVGKSR

SEQ ID NO: 1070 - protein, 9 aa, synthetic construct
  source 1..9: note = Seed: AGDVGKSAL (KRAS G13D)
AGDVGKSAA

SEQ ID NO: 1071 - protein, 9 aa, synthetic construct
  source 1..9: note = Seed: VVGARGVGK (KRAS G12R)
VAGARGVGW

SEQ ID NO: 1072 - protein, 9 aa, synthetic construct
  source 1..9: note = Seed: VVGADGVGK (KRAS G12D)
VTGADGVGM

SEQ ID NO: 1073 - protein, 9 aa, synthetic construct
  source 1..9: note = Seed: LVVVGACGV (KRAS G12C)
LMVVGACGT

SEQ ID NO: 1074 - protein, 9 aa, synthetic construct
  source 1..9: note = Seed: GADGVGKSA (KRAS G12D)
GPDGVGKSK

SEQ ID NO: 1075 - protein, 9 aa, synthetic construct
  source 1..9: note = Seed: GACGVGKSA (KRAS G12C)
GECGVGKSF

SEQ ID NO: 1076 - protein, 9 aa, synthetic construct
  source 1..9: note = Seed: GADGVGKSA (KRAS G12D)
GVDGVGKSH

SEQ ID NO: 1077 - protein, 9 aa, synthetic construct
  source 1..9: note = Seed: AGDVGKSAL (KRAS G13D)
ALDVGKSAR

SEQ ID NO: 1078 - protein, 9 aa, synthetic construct
  source 1..9: note = Seed: AGDVGKSAL (KRAS G13D)
ATDVGKSAK

SEQ ID NO: 1079 - protein, 9 aa, synthetic construct
  source 1..9: note = Seed: GAGDVGKSA (KRAS G13D)
GVGDVGKSA

SEQ ID NO: 1080 - protein, 9 aa, synthetic construct
  source 1..9: note = Seed: GADGVGKSA (KRAS G12D)
GRDGVGKSA

SEQ ID NO: 1081 - protein, 9 aa, synthetic construct
  source 1..9: note = Seed: GACGVGKSA (KRAS G12C)
GLCGVGKSK

SEQ ID NO: 1082 - protein, 9 aa, synthetic construct
  source 1..9: note = Seed: AGDVGKSAL (KRAS G13D)
AKDVGKSAI

SEQ ID NO: 1083 - protein, 9 aa, synthetic construct
  source 1..9: note = Seed: AGDVGKSAL (KRAS G13D)
ARDVGKSAA

SEQ ID NO: 1084 - protein, 9 aa, synthetic construct
  source 1..9: note = Seed: GAVGVGKSA (KRAS G12V)
GIVGVGKSV

SEQ ID NO: 1085 - protein, 9 aa, synthetic construct
  source 1..9: note = Seed: GADGVGKSA (KRAS G12D)
GDDGVGKSV

SEQ ID NO: 1086 - protein, 9 aa, synthetic construct
  source 1..9: note = Seed: GADGVGKSA (KRAS G12D)
GEDGVGKSY

SEQ ID NO: 1087 - protein, 9 aa, synthetic construct
  source 1..9: note = Seed: DVGKSALTI (KRAS G13D)
DGGKSALTL

SEQ ID NO: 1088 - protein, 9 aa, synthetic construct
  source 1..9: note = Seed: VVGACGVGK (KRAS G12C)
VYGACGVGK

SEQ ID NO: 1089 - protein, 9 aa, synthetic construct
  source 1..9: note = Seed: GAVGVGKSA (KRAS G12V)
GIVGVGKSI

SEQ ID NO: 1090 - protein, 9 aa, synthetic construct
  source 1..9: note = Seed: GAVGVGKSA (KRAS G12V)
GFVGVGKSK

SEQ ID NO: 1091 - protein, 9 aa, synthetic construct
  source 1..9: note = Seed: VVGACGVGK (KRAS G12C)
VNGACGVGK

SEQ ID NO: 1092 - protein, 9 aa, synthetic construct
  source 1..9: note = Seed: GAGDVGKSA (KRAS G13D)
GFGDVGKSK

SEQ ID NO: 1093 - protein, 9 aa, synthetic construct
  source 1..9: note = Seed: AGDVGKSAL (KRAS G13D)
ATDVGKSAR

SEQ ID NO: 1094 - protein, 9 aa, synthetic construct
  source 1..9: note = Seed: VVGAVGVGK (KRAS G12V)
VCGAVGVGK

SEQ ID NO: 1095 - protein, 9 aa, synthetic construct
  source 1..9: note = Seed: GADGVGKSA (KRAS G12D)
GRDGVGKSC

SEQ ID NO: 1096 - protein, 9 aa, synthetic construct
  source 1..9: note = Seed: GAVGVGKSA (KRAS G12V)
GAVGVGKSQ

SEQ ID NO: 1097 - protein, 9 aa, synthetic construct
  source 1..9: note = Seed: VVGAVGVGK (KRAS G12V)
VYGAVGVGF

SEQ ID NO: 1098 - protein, 9 aa, synthetic construct
  source 1..9: note = Seed: GAVGVGKSA (KRAS G12V)
GCVGVGKSK

SEQ ID NO: 1099 - protein, 9 aa, synthetic construct
  source 1..9: note = Seed: VVGARGVGK (KRAS G12R)
VGGARGVGK

SEQ ID NO: 1100 - protein, 9 aa, synthetic construct
  source 1..9: note = Seed: VVGADGVGK (KRAS G12D)
VDGADGVGR

SEQ ID NO: 1101 - protein, 9 aa, synthetic construct
  source 1..9: note = Seed: VVGACGVGK (KRAS G12C)
VDGACGVGR

SEQ ID NO: 1102 - protein, 9 aa, synthetic construct
  source 1..9: note = Seed: GADGVGKSA (KRAS G12D)
GYDGVGKSK

SEQ ID NO: 1103 - protein, 9 aa, synthetic construct
  source 1..9: note = Seed: AGDVGKSAL (KRAS G13D)
ALDVGKSAK

SEQ ID NO: 1104 - protein, 9 aa, synthetic construct
  source 1..9: note = Seed: VVGAGDVGK (KRAS G13D)
VFGAGDVGR

SEQ ID NO: 1105 - protein, 9 aa, synthetic construct
  source 1..9: note = Seed: VVGAGDVGK (KRAS G13D)
VEGAGDVGK

SEQ ID NO: 1106 - protein, 9 aa, synthetic construct
  source 1..9: note = Seed: VVGADGVGK (KRAS G12D)
VFGADGVGR

SEQ ID NO: 1107 - protein, 9 aa, synthetic construct
  source 1..9: note = Seed: AGDVGKSAL (KRAS G13D)
ALDVGKSAA

SEQ ID NO: 1108 - protein, 9 aa, synthetic construct
  source 1..9: note = Seed: GADGVGKSA (KRAS G12D)
GSDGVGKSH

SEQ ID NO: 1109 - protein, 9 aa, synthetic construct
  source 1..9: note = Seed: VVGAVGVGK (KRAS G12V)
VRGAVGVGF

SEQ ID NO: 1110 - protein, 9 aa, synthetic construct
  source 1..9: note = Seed: VVGAVGVGK (KRAS G12V)
VNGAVGVGY

SEQ ID NO: 1111 - protein, 9 aa, synthetic construct
  source 1..9: note = Seed: GAGDVGKSA (KRAS G13D)
GIGDVGKSV

SEQ ID NO: 1112 - protein, 9 aa, synthetic construct
  source 1..9: note = Seed: AGDVGKSAL (KRAS G13D)
ARDVGKSAC

SEQ ID NO: 1113 - protein, 9 aa, synthetic construct
  source 1..9: note = Seed: GAVGVGKSA (KRAS G12V)
GCVGVGKSL

SEQ ID NO: 1114 - protein, 9 aa, synthetic construct
  source 1..9: note = Seed: AGDVGKSAL (KRAS G13D)
AEDVGKSAT

SEQ ID NO: 1115 - protein, 9 aa, synthetic construct
  source 1..9: note = Seed: GAVGVGKSA (KRAS G12V)
GPVGVGKSK

SEQ ID NO: 1116 - protein, 9 aa, synthetic construct
  source 1..9: note = Seed: GAVGVGKSA (KRAS G12V)
GTVGVGKSW

SEQ ID NO: 1117 - protein, 9 aa, synthetic construct
  source 1..9: note = Seed: VVGACGVGK (KRAS G12C)
VEGACGVGK

SEQ ID NO: 1118 - protein, 9 aa, synthetic construct
  source 1..9: note = Seed: GACGVGKSA (KRAS G12C)
GDCGVGKSY

SEQ ID NO: 1119 - protein, 9 aa, synthetic construct
  source 1..9: note = Seed: DVGKSALTI (KRAS G13D)
DEGKSALTV

SEQ ID NO: 1120 - protein, 9 aa, synthetic construct
  source 1..9: note = Seed: DVGKSALTI (KRAS G13D)
DQGKSALTM

SEQ ID NO: 1121 - protein, 9 aa, synthetic construct
  source 1..9: note = Seed: GADGVGKSA (KRAS G12D)
GEDGVGKSK

SEQ ID NO: 1122 - protein, 9 aa, synthetic construct
  source 1..9: note = Seed: VVGACGVGK (KRAS G12C)
VFGACGVGK

SEQ ID NO: 1123 - protein, 9 aa, synthetic construct
  source 1..9: note = Seed: VVGAGDVGK (KRAS G13D)
VGGAGDVGY

SEQ ID NO: 1124 - protein, 9 aa, synthetic construct
  source 1..9: note = Seed: GAVGVGKSA (KRAS G12V)
GCVGVGKSR

SEQ ID NO: 1125 - protein, 9 aa, synthetic construct
  source 1..9: note = Seed: GADGVGKSA (KRAS G12D)
GFDGVGKSR

SEQ ID NO: 1126 - protein, 9 aa, synthetic construct
  source 1..9: note = Seed: VVGAGDVGK (KRAS G13D)
VDGAGDVGR

SEQ ID NO: 1127 - protein, 9 aa, synthetic construct
  source 1..9: note = Seed: VVGAVGVGK (KRAS G12V)
VAGAVGVGW

SEQ ID NO: 1128 - protein, 9 aa, synthetic construct
  source 1..9: note = Seed: GADGVGKSA (KRAS G12D)
GFDGVGKSW

SEQ ID NO: 1129 - protein, 9 aa, synthetic construct
  source 1..9: note = Seed: VVGAVGVGK (KRAS G12V)
VRGAVGVGY

SEQ ID NO: 1130 - protein, 9 aa, synthetic construct
  source 1..9: note = Seed: VVGAVGVGK (KRAS G12V)
VIGAVGVGL

SEQ ID NO: 1131 - protein, 9 aa, synthetic construct
  source 1..9: note = Seed: GACGVGKSA (KRAS G12C)
GICGVGKSV

SEQ ID NO: 1132 - protein, 9 aa, synthetic construct
  source 1..9: note = Seed: VVGACGVGK (KRAS G12C)
VTGACGVGM

SEQ ID NO: 1133 - protein, 9 aa, synthetic construct
  source 1..9: note = Seed: AGDVGKSAL (KRAS G13D)
ACDVGKSAY

SEQ ID NO: 1134 - protein, 9 aa, synthetic construct
  source 1..9: note = Seed: VVGAVGVGK (KRAS G12V)
VCGAVGVGR

SEQ ID NO: 1135 - protein, 9 aa, synthetic construct
  source 1..9: note = Seed: VVGADGVGK (KRAS G12D)
VEGADGVGR

SEQ ID NO: 1136 - protein, 9 aa, synthetic construct
  source 1..9: note = Seed: AGDVGKSAL (KRAS G13D)
AVDVGKSAH

SEQ ID NO: 1137 - protein, 9 aa, synthetic construct
  source 1..9: note = Seed: GAGDVGKSA (KRAS G13D)
GDGDVGKSY

SEQ ID NO: 1138 - protein, 9 aa, synthetic construct
  source 1..9: note = Seed: DVGKSALTI (KRAS G13D)
DPGKSALTM

SEQ ID NO: 1139 - protein, 9 aa, synthetic construct
  source 1..9: note = Seed: GACGVGKSA (KRAS G12C)
GSCGVGKSA

SEQ ID NO: 1140 - protein, 9 aa, synthetic construct
  source 1..9: note = Seed: GAGDVGKSA (KRAS G13D)
GSGDVGKSW

SEQ ID NO: 1141 - protein, 9 aa, synthetic construct
  source 1..9: note = Seed: GACGVGKSA (KRAS G12C)
GDCGVGKSK

SEQ ID NO: 1142 - protein, 9 aa, synthetic construct
  source 1..9: note = Seed: VVGAVGVGK (KRAS G12V)
VYGAVGVGM

SEQ ID NO: 1143 - protein, 9 aa, synthetic construct
  source 1..9: note = Seed: VVGACGVGK (KRAS G12C)
VWGACGVGR

SEQ ID NO: 1144 - protein, 9 aa, synthetic construct
  source 1..9: note = Seed: GAVGVGKSA (KRAS G12V)
GTVGVGKSA

SEQ ID NO: 1145 - protein, 9 aa, synthetic construct
  source 1..9: note = Seed: GAGDVGKSA (KRAS G13D)
GSGDVGKSA

SEQ ID NO: 1146 - protein, 9 aa, synthetic construct
  source 1..9: note = Seed: DVGKSALTI (KRAS G13D)
DLGKSALTV

SEQ ID NO: 1147 - protein, 9 aa, synthetic construct
  source 1..9: note = Seed: AGDVGKSAL (KRAS G13D)
AQDVGKSAK

SEQ ID NO: 1148 - protein, 9 aa, synthetic construct
  source 1..9: note = Seed: AGDVGKSAL (KRAS G13D)
ATDVGKSAT

SEQ ID NO: 1149 - protein, 9 aa, synthetic construct
  source 1..9: note = Seed: VVGACGVGK (KRAS G12C)
VNGACGVGR

SEQ ID NO: 1150 - protein, 9 aa, synthetic construct
  source 1..9: note = Seed: GACGVGKSA (KRAS G12C)
GQCGVGKSK

SEQ ID NO: 1151 - protein, 9 aa, synthetic construct
  source 1..9: note = Seed: LVVVGAVGV (KRAS G12V)
LMVVGAVGT

SEQ ID NO: 1152 - protein, 9 aa, synthetic construct
  source 1..9: note = Seed: VVGARGVGK (KRAS G12R)
VIGARGVGL

SEQ ID NO: 1153 - protein, 9 aa, synthetic construct
  source 1..9: note = Seed: VVGAVGVGK (KRAS G12V)
VYGAVGVGY

SEQ ID NO: 1154 - protein, 9 aa, synthetic construct
  source 1..9: note = Seed: GACGVGKSA (KRAS G12C)
GNCGVGKSK

SEQ ID NO: 1155 - protein, 9 aa, synthetic construct
  source 1..9: note = Seed: VVGACGVGK (KRAS G12C)
VTGACGVGL

SEQ ID NO: 1156 - protein, 9 aa, synthetic construct
  source 1..9: note = Seed: VVGAVGVGK (KRAS G12V)
VHGAVGVGK

SEQ ID NO: 1157 - protein, 9 aa, synthetic construct
  source 1..9: note = Seed: VVGARGVGK (KRAS G12R)
VGGARGVGM

SEQ ID NO: 1158 - protein, 9 aa, synthetic construct
  source 1..9: note = Seed: VVGAGDVGK (KRAS G13D)
VPGAGDVGK

SEQ ID NO: 1159 - protein, 9 aa, synthetic construct
  source 1..9: note = Seed: GAGDVGKSA (KRAS G13D)
GPGDVGKSF

SEQ ID NO: 1160 - protein, 9 aa, synthetic construct
  source 1..9: note = Seed: VVGARGVGK (KRAS G12R)
VIGARGVGM

SEQ ID NO: 1161 - protein, 9 aa, synthetic construct
  source 1..9: note = Seed: GARGVGKSA (KRAS G12R)
GPRGVGKSR

SEQ ID NO: 1162 - protein, 9 aa, synthetic construct
  source 1..9: note = Seed: VVGARGVGK (KRAS G12R)
VFGARGVGY

SEQ ID NO: 1163 - protein, 9 aa, synthetic construct
  source 1..9: note = Seed: VVGACGVGK (KRAS G12C)
VQGACGVGY

SEQ ID NO: 1164 - protein, 9 aa, synthetic construct
  source 1..9: note = Seed: AGDVGKSAL (KRAS G13D)
ADDVGKSAK

SEQ ID NO: 1165 - protein, 9 aa, synthetic construct
  source 1..9: note = Seed: VVGADGVGK (KRAS G12D)
VSGADGVGL

SEQ ID NO: 1166 - protein, 9 aa, synthetic construct
  source 1..9: note = Seed: GADGVGKSA (KRAS G12D)
GLDGVGKSA

SEQ ID NO: 1167 - protein, 9 aa, synthetic construct
  source 1..9: note = Seed: VVGAGDVGK (KRAS G13D)
VYGAGDVGK

SEQ ID NO: 1168 - protein, 9 aa, synthetic construct
  source 1..9: note = Seed: VVGARGVGK (KRAS G12R)
VGGARGVGF

SEQ ID NO: 1169 - protein, 9 aa, synthetic construct
  source 1..9: note = Seed: GAGDVGKSA (KRAS G13D)
GVGDVGKSW

SEQ ID NO: 1170 - protein, 9 aa, synthetic construct
  source 1..9: note = Seed: VVGAVGVGK (KRAS G12V)
VCGAVGVGY

SEQ ID NO: 1171 - protein, 9 aa, synthetic construct
  source 1..9: note = Seed: VVGAGDVGK (KRAS G13D)
VVGAGDVGM

SEQ ID NO: 1172 - protein, 9 aa, synthetic construct
  source 1..9: note = Seed: VVGARGVGK (KRAS G12R)
VGGARGVGR

SEQ ID NO: 1173 - protein, 9 aa, synthetic construct
  source 1..9: note = Seed: GAGDVGKSA (KRAS G13D)
GNGDVGKSM

SEQ ID NO: 1174 - protein, 9 aa, synthetic construct
  source 1..9: note = Seed: GADGVGKSA (KRAS G12D)
GKDGVGKSI

SEQ ID NO: 1175 - protein, 9 aa, synthetic construct
  source 1..9: note = Seed: VVGARGVGK (KRAS G12R)
VMGARGVGY

SEQ ID NO: 1176 - protein, 9 aa, synthetic construct
  source 1..9: note = Seed: VVGARGVGK (KRAS G12R)
VPGARGVGK

SEQ ID NO: 1177 - protein, 9 aa, synthetic construct
  source 1..9: note = Seed: GAVGVGKSA (KRAS G12V)
GVVGVGKSW

SEQ ID NO: 1178 - protein, 9 aa, synthetic construct
  source 1..9: note = Seed: VVGACGVGK (KRAS G12C)
VAGACGVGI

SEQ ID NO: 1179 - protein, 9 aa, synthetic construct
  source 1..9: note = Seed: GARGVGKSA (KRAS G12R)
GCRGVGKSM

SEQ ID NO: 1180 - protein, 9 aa, synthetic construct
  source 1..9: note = Seed: AGDVGKSAL (KRAS G13D)
AMDVGKSAW

SEQ ID NO: 1181 - protein, 9 aa, synthetic construct
  source 1..9: note = Seed: AGDVGKSAL (KRAS G13D)
ANDVGKSAK

SEQ ID NO: 1182 - protein, 9 aa, synthetic construct
  source 1..9: note = Seed: GAVGVGKSA (KRAS G12V)
GAVGVGKSS

SEQ ID NO: 1183 - protein, 9 aa, synthetic construct
  source 1..9: note = Seed: GADGVGKSA (KRAS G12D)
GEDGVGKSI

SEQ ID NO: 1184 - protein, 9 aa, synthetic construct
  source 1..9: note = Seed: AGDVGKSAL (KRAS G13D)
ANDVGKSAR

SEQ ID NO: 1185 - protein, 9 aa, synthetic construct
  source 1..9: note = Seed: GADGVGKSA (KRAS G12D)
GCDGVGKSY

SEQ ID NO: 1186 - protein, 9 aa, synthetic construct
  source 1..9: note = Seed: GAGDVGKSA (KRAS G13D)
GEGDVGKSK

SEQ ID NO: 1187 - protein, 9 aa, synthetic construct
  source 1..9: note = Seed: AGDVGKSAL (KRAS G13D)
AQDVGKSAR

SEQ ID NO: 1188 - protein, 9 aa, synthetic construct
  source 1..9: note = Seed: AGDVGKSAL (KRAS G13D)
AGDVGKSAW

SEQ ID NO: 1189 - protein, 9 aa, synthetic construct
  source 1..9: note = Seed: AGDVGKSAL (KRAS G13D)
AGDVGKSAC

SEQ ID NO: 1190 - protein, 9 aa, synthetic construct
  source 1..9: note = Seed: GAGDVGKSA (KRAS G13D)
GPGDVGKSG

SEQ ID NO: 1191 - protein, 9 aa, synthetic construct
  source 1..9: note = Seed: GAGDVGKSA (KRAS G13D)
GAGDVGKSC

SEQ ID NO: 1192 - protein, 9 aa, synthetic construct
  source 1..9: note = Seed: GARGVGKSA (KRAS G12R)
GGRGVGKSH

SEQ ID NO: 1193 - protein, 9 aa, synthetic construct
  source 1..9: note = Seed: GARGVGKSA (KRAS G12R)
GSRGVGKSW

SEQ ID NO: 1194 - protein, 9 aa, synthetic construct
  source 1..9: note = Seed: GARGVGKSA (KRAS G12R)
GDRGVGKSF

SEQ ID NO: 1195 - protein, 9 aa, synthetic construct
  source 1..9: note = Seed: GACGVGKSA (KRAS G12C)
GNCGVGKSR

SEQ ID NO: 1196 - protein, 9 aa, synthetic construct
  source 1..9: note = Seed: VVGAGDVGK (KRAS G13D)
VEGAGDVGR

SEQ ID NO: 1197 - protein, 9 aa, synthetic construct
  source 1..9: note = Seed: VVGARGVGK (KRAS G12R)
VGGARGVGL

SEQ ID NO: 1198 - protein, 9 aa, synthetic construct
  source 1..9: note = Seed: GAGDVGKSA (KRAS G13D)
GFGDVGKSR

SEQ ID NO: 1199 - protein, 9 aa, synthetic construct
  source 1..9: note = Seed: GAGDVGKSA (KRAS G13D)
GPGDVGKSK

SEQ ID NO: 1200 - protein, 9 aa, synthetic construct
  source 1..9: note = Seed: GACGVGKSA (KRAS G12C)
GFCGVGKSY

SEQ ID NO: 1201 - protein, 9 aa, synthetic construct
  source 1..9: note = Seed: VVGADGVGK (KRAS G12D)
VYGADGVGK

SEQ ID NO: 1202 - protein, 9 aa, synthetic construct
  source 1..9: note = Seed: GADGVGKSA (KRAS G12D)
GIDGVGKST

SEQ ID NO: 1203 - protein, 9 aa, synthetic construct
  source 1..9: note = Seed: VVGAGDVGK (KRAS G13D)
VSGAGDVGL

SEQ ID NO: 1204 - protein, 9 aa, synthetic construct
  source 1..9: note = Seed: VVGARGVGK (KRAS G12R)
VWGARGVGR

SEQ ID NO: 1205 - protein, 9 aa, synthetic construct
  source 1..9: note = Seed: GADGVGKSA (KRAS G12D)
GVDGVGKST

SEQ ID NO: 1206 - protein, 9 aa, synthetic construct
  source 1..9: note = Seed: GADGVGKSA (KRAS G12D)
GPDGVGKSS

SEQ ID NO: 1207 - protein, 9 aa, synthetic construct
  source 1..9: note = Seed: GACGVGKSA (KRAS G12C)
GNCGVGKSM

SEQ ID NO: 1208 - protein, 9 aa, synthetic construct
  source 1..9: note = Seed: GADGVGKSA (KRAS G12D)
GEDGVGKSR

SEQ ID NO: 1209 - protein, 9 aa, synthetic construct
  source 1..9: note = Seed: GACGVGKSA (KRAS G12C)
GQCGVGKSR

SEQ ID NO: 1210 - protein, 9 aa, synthetic construct
  source 1..9: note = Seed: GADGVGKSA (KRAS G12D)
GPDGVGKSY

SEQ ID NO: 1211 - protein, 9 aa, synthetic construct
  source 1..9: note = Seed: VVGACGVGK (KRAS G12C)
VEGACGVGR

SEQ ID NO: 1212 - protein, 9 aa, synthetic construct
  source 1..9: note = Seed: VVGAVGVGK (KRAS G12V)
VLGAVGVGF

SEQ ID NO: 1213 - protein, 9 aa, synthetic construct
  source 1..9: note = Seed: GAVGVGKSA (KRAS G12V)
GPVGVGKSW

SEQ ID NO: 1214 - protein, 9 aa, synthetic construct
  source 1..9: note = Seed: GADGVGKSA (KRAS G12D)
GYDGVGKSW

SEQ ID NO: 1215 - protein, 9 aa, synthetic construct
  source 1..9: note = Seed: GACGVGKSA (KRAS G12C)
GICGVGKSI

SEQ ID NO: 1216 - protein, 9 aa, synthetic construct
  source 1..9: note = Seed: GARGVGKSA (KRAS G12R)
GTRGVGKSA

SEQ ID NO: 1217 - protein, 9 aa, synthetic construct
  source 1..9: note = Seed: VVGAVGVGK (KRAS G12V)
VWGAVGVGK

SEQ ID NO: 1218 - protein, 9 aa, synthetic construct
  source 1..9: note = Seed: VVGARGVGK (KRAS G12R)
VFGARGVGF

SEQ ID NO: 1219 - protein, 9 aa, synthetic construct
  source 1..9: note = Seed: GAVGVGKSA (KRAS G12V)
GPVGVGKSR

SEQ ID NO: 1220 - protein, 9 aa, synthetic construct
  source 1..9: note = Seed: VVGAVGVGK (KRAS G12V)
VDGAVGVGY

SEQ ID NO: 1221 - protein, 9 aa, synthetic construct
  source 1..9: note = Seed: GAGDVGKSA (KRAS G13D)
GGGDVGKSH

SEQ ID NO: 1222 - protein, 9 aa, synthetic construct
  source 1..9: note = Seed: GACGVGKSA (KRAS G12C)
GGCGVGKSH

SEQ ID NO: 1223 - protein, 9 aa, synthetic construct
  source 1..9: note = Seed: VVGAVGVGK (KRAS G12V)
VFGAVGVGF

SEQ ID NO: 1224 - protein, 9 aa, synthetic construct
  source 1..9: note = Seed: VVGAGDVGK (KRAS G13D)
VIGAGDVGF

SEQ ID NO: 1225 - protein, 9 aa, synthetic construct
  source 1..9: note = Seed: AGDVGKSAL (KRAS G13D)
AKDVGKSAV

SEQ ID NO: 1226 - protein, 9 aa, synthetic construct
  source 1..9: note = Seed: GACGVGKSA (KRAS G12C)
GDCGVGKSR

SEQ ID NO: 1227 - protein, 9 aa, synthetic construct
  source 1..9: note = Seed: VVGARGVGK (KRAS G12R)
VDGARGVGK

SEQ ID NO: 1228 - protein, 9 aa, synthetic construct
  source 1..9: note = Seed: GARGVGKSA (KRAS G12R)
GPRGVGKSE

SEQ ID NO: 1229 - protein, 9 aa, synthetic construct
  source 1..9: note = Seed: AGDVGKSAL (KRAS G13D)
APDVGKSAY

SEQ ID NO: 1230 - protein, 9 aa, synthetic construct
  source 1..9: note = Seed: VVGAVGVGK (KRAS G12V)
VYGAVGVGL

SEQ ID NO: 1231 - protein, 9 aa, synthetic construct
  source 1..9: note = Seed: GAGDVGKSA (KRAS G13D)
GMGDVGKSH

SEQ ID NO: 1232 - protein, 9 aa, synthetic construct
  source 1..9: note = Seed: VVGADGVGK (KRAS G12D)
VTGADGVGL

SEQ ID NO: 1233 - protein, 9 aa, synthetic construct
  source 1..9: note = Seed: GAGDVGKSA (KRAS G13D)
GAGDVGKST

SEQ ID NO: 1234 - protein, 9 aa, synthetic construct
  source 1..9: note = Seed: GARGVGKSA (KRAS G12R)
GPRGVGKSK

SEQ ID NO: 1235 - protein, 9 aa, synthetic construct
  source 1..9: note = Seed: GADGVGKSA (KRAS G12D)
GCDGVGKSK

SEQ ID NO: 1236 - protein, 9 aa, synthetic construct
  source 1..9: note = Seed: GADGVGKSA (KRAS G12D)
GGDGVGKSC

SEQ ID NO: 1237 - protein, 9 aa, synthetic construct
  source 1..9: note = Seed: AGDVGKSAL (KRAS G13D)
ATDVGKSAH

SEQ ID NO: 1238 - protein, 9 aa, synthetic construct
  source 1..9: note = Seed: VVGADGVGK (KRAS G12D)
VQGADGVGY

SEQ ID NO: 1239 - protein, 9 aa, synthetic construct
  source 1..9: note = Seed: VVGAGDVGK (KRAS G13D)
VTGAGDVGM

SEQ ID NO: 1240 - protein, 9 aa, synthetic construct
  source 1..9: note = Seed: GAGDVGKSA (KRAS G13D)
GIGDVGKSI

SEQ ID NO: 1241 - protein, 9 aa, synthetic construct
  source 1..9: note = Seed: GACGVGKSA (KRAS G12C)
GMCGVGKSR

SEQ ID NO: 1242 - protein, 9 aa, synthetic construct
  source 1..9: note = Seed: AGDVGKSAL (KRAS G13D)
AMDVGKSAR

SEQ ID NO: 1243 - protein, 9 aa, synthetic construct
  source 1..9: note = Seed: GACGVGKSA (KRAS G12C)
GCCGVGKSF

SEQ ID NO: 1244 - protein, 9 aa, synthetic construct
  source 1..9: note = Seed: AGDVGKSAL (KRAS G13D)
AEDVGKSAK

SEQ ID NO: 1245 - protein, 9 aa, synthetic construct
  source 1..9: note = Seed: GAVGVGKSA (KRAS G12V)
GEVGVGKSR

SEQ ID NO: 1246 - protein, 9 aa, synthetic construct
  source 1..9: note = Seed: GADGVGKSA (KRAS G12D)
GWDGVGKSK

SEQ ID NO: 1247 - protein, 9 aa, synthetic construct
  source 1..9: note = Seed: GACGVGKSA (KRAS G12C)
GSCGVGKSW

SEQ ID NO: 1248 - protein, 9 aa, synthetic construct
  source 1..9: note = Seed: VVGACGVGK (KRAS G12C)
VVGACGVGL

SEQ ID NO: 1249 - protein, 9 aa, synthetic construct
  source 1..9: note = Seed: VVGAVGVGK (KRAS G12V)
VSGAVGVGI

SEQ ID NO: 1250 - protein, 9 aa, synthetic construct
  source 1..9: note = Seed: AGDVGKSAL (KRAS G13D)
AIDVGKSAT

SEQ ID NO: 1251 - protein, 9 aa, synthetic construct
  source 1..9: note = Seed: GADGVGKSA (KRAS G12D)
GTDGVGKSH

SEQ ID NO: 1252 - protein, 9 aa, synthetic construct
  source 1..9: note = Seed: GARGVGKSA (KRAS G12R)
GARGVGKSR

SEQ ID NO: 1253 - protein, 9 aa, synthetic construct
  source 1..9: note = Seed: VVGAVGVGK (KRAS G12V)
VSGAVGVGV

SEQ ID NO: 1254 - protein, 9 aa, synthetic construct
  source 1..9: note = Seed: VVGARGVGK (KRAS G12R)
VYGARGVGK

SEQ ID NO: 1255 - protein, 9 aa, synthetic construct
  source 1..9: note = Seed: AGDVGKSAL (KRAS G13D)
AMDVGKSAA

SEQ ID NO: 1256 - protein, 9 aa, synthetic construct
  source 1..9: note = Seed: VVGARGVGK (KRAS G12R)
VLGARGVGY

SEQ ID NO: 1257 - protein, 9 aa, synthetic construct
  source 1..9: note = Seed: GARGVGKSA (KRAS G12R)
GVRGVGKSW

SEQ ID NO: 1258 - protein, 9 aa, synthetic construct
  source 1..9: note = Seed: GACGVGKSA (KRAS G12C)
GACGVGKST

SEQ ID NO: 1259 - protein, 9 aa, synthetic construct
  source 1..9: note = Seed: GADGVGKSA (KRAS G12D)
GYDGVGKSR

SEQ ID NO: 1260 - protein, 9 aa, synthetic construct
  source 1..9: note = Seed: GADGVGKSA (KRAS G12D)
GCDGVGKSR

SEQ ID NO: 1261 - protein, 9 aa, synthetic construct
  source 1..9: note = Seed: GAVGVGKSA (KRAS G12V)
GGVGVGKSV

SEQ ID NO: 1262 - protein, 9 aa, synthetic construct
  source 1..9: note = Seed: VVGAVGVGK (KRAS G12V)
VFGAVGVGY

SEQ ID NO: 1263 - protein, 9 aa, synthetic construct
  source 1..9: note = Seed: VVGAVGVGK (KRAS G12V)
VIGAVGVGH

SEQ ID NO: 1264 - protein, 9 aa, synthetic construct
  source 1..9: note = Seed: VVGACGVGK (KRAS G12C)
VAGACGVGV

SEQ ID NO: 1265 - protein, 9 aa, synthetic construct
  source 1..9: note = Seed: AGDVGKSAL (KRAS G13D)
ADDVGKSAR

SEQ ID NO: 1266 - protein, 9 aa, synthetic construct
  source 1..9: note = Seed: DVGKSALTI (KRAS G13D)
DLGKSALTI

SEQ ID NO: 1267 - protein, 9 aa, synthetic construct
  source 1..9: note = Seed: AGDVGKSAL (KRAS G13D)
AQDVGKSAC

SEQ ID NO: 1268 - protein, 9 aa, synthetic construct
  source 1..9: note = Seed: GADGVGKSA (KRAS G12D)
GKDGVGKSV

SEQ ID NO: 1269 - protein, 9 aa, synthetic construct
  source 1..9: note = Seed: GAGDVGKSA (KRAS G13D)
GCGDVGKSR

SEQ ID NO: 1270 - protein, 9 aa, synthetic construct
  source 1..9: note = Seed: GADGVGKSA (KRAS G12D)
GPDGVGKSR

SEQ ID NO: 1271 - protein, 9 aa, synthetic construct
  source 1..9: note = Seed: LVVVGAVGV (KRAS G12V)
LAVVGAVGL

SEQ ID NO: 1272 - protein, 9 aa, synthetic construct
  source 1..9: note = Seed: GAGDVGKSA (KRAS G13D)
GCGDVGKSK

SEQ ID NO: 1273 - protein, 9 aa, synthetic construct
  source 1..9: note = Seed: GAGDVGKSA (KRAS G13D)
GLGDVGKSH

SEQ ID NO: 1274 - protein, 9 aa, synthetic construct
  source 1..9: note = Seed: LVVVGARGV (KRAS G12R)
LMVVGARGM

SEQ ID NO: 1275 - protein, 9 aa, synthetic construct
  source 1..9: note = Seed: LVVVGARGV (KRAS G12R)
LLVVGARGM

SEQ ID NO: 1276 - protein, 9 aa, synthetic construct
  source 1..9: note = Seed: AGDVGKSAL (KRAS G13D)
AMDVGKSAK

SEQ ID NO: 1277 - protein, 9 aa, synthetic construct
  source 1..9: note = Seed: LVVVGACGV (KRAS G12C)
LLVVGACGT

SEQ ID NO: 1278 - protein, 9 aa, synthetic construct
  source 1..9: note = Seed: GARGVGKSA (KRAS G12R)
GARGVGKSK

SEQ ID NO: 1279 - protein, 9 aa, synthetic construct
  source 1..9: note = Seed: VVGAGDVGK (KRAS G13D)
VTGAGDVGL

SEQ ID NO: 1280 - protein, 9 aa, synthetic construct
  source 1..9: note = Seed: GADGVGKSA (KRAS G12D)
GMDGVGKSW

SEQ ID NO: 1281 - protein, 9 aa, synthetic construct
  source 1..9: note = Seed: VVGARGVGK (KRAS G12R)
VDGARGVGR

SEQ ID NO: 1282 - protein, 9 aa, synthetic construct
  source 1..9: note = Seed: GAVGVGKSA (KRAS G12V)
GFVGVGKSR

SEQ ID NO: 1283 - protein, 9 aa, synthetic construct
  source 1..9: note = Seed: GADGVGKSA (KRAS G12D)
GRDGVGKSY

SEQ ID NO: 1284 - protein, 9 aa, synthetic construct
  source 1..9: note = Seed: DVGKSALTI (KRAS G13D)
DIGKSALTL

SEQ ID NO: 1285 - protein, 9 aa, synthetic construct
  source 1..9: note = Seed: GAVGVGKSA (KRAS G12V)
GFVGVGKSF

SEQ ID NO: 1286 - protein, 9 aa, synthetic construct
  source 1..9: note = Seed: GADGVGKSA (KRAS G12D)
GADGVGKSS

SEQ ID NO: 1287 - protein, 9 aa, synthetic construct
  source 1..9: note = Seed: GAGDVGKSA (KRAS G13D)
GCGDVGKSM

SEQ ID NO: 1288 - protein, 9 aa, synthetic construct
  source 1..9: note = Seed: VVGADGVGK (KRAS G12D)
VSGADGVGH

SEQ ID NO: 1289 - protein, 9 aa, synthetic construct
  source 1..9: note = Seed: GAGDVGKSA (KRAS G13D)
GYGDVGKSY

SEQ ID NO: 1290 - protein, 9 aa, synthetic construct
  source 1..9: note = Seed: GAVGVGKSA (KRAS G12V)
GYVGVGKSK

SEQ ID NO: 1291 - protein, 9 aa, synthetic construct
  source 1..9: note = Seed: GACGVGKSA (KRAS G12C)
GFCGVGKSK

SEQ ID NO: 1292 - protein, 9 aa, synthetic construct
  source 1..9: note = Seed: GACGVGKSA (KRAS G12C)
GACGVGKSC

SEQ ID NO: 1293 - protein, 9 aa, synthetic construct
  source 1..9: note = Seed: VVGACGVGK (KRAS G12C)
VGGACGVGF

SEQ ID NO: 1294 - protein, 9 aa, synthetic construct
  source 1..9: note = Seed: GAGDVGKSA (KRAS G13D)
GFGDVGKSF

SEQ ID NO: 1295 - protein, 9 aa, synthetic construct
  source 1..9: note = Seed: GADGVGKSA (KRAS G12D)
GGDGVGKSA

SEQ ID NO: 1296 - protein, 9 aa, synthetic construct
  source 1..9: note = Seed: GADGVGKSA (KRAS G12D)
GWDGVGKSR

SEQ ID NO: 1297 - protein, 9 aa, synthetic construct
  source 1..9: note = Seed: GACGVGKSA (KRAS G12C)
GMCGVGKSK

SEQ ID NO: 1298 - protein, 9 aa, synthetic construct
  source 1..9: note = Seed: GAGDVGKSA (KRAS G13D)
GYGDVGKSK

SEQ ID NO: 1299 - protein, 9 aa, synthetic construct
  source 1..9: note = Seed: GACGVGKSA (KRAS G12C)
GECGVGKSK

SEQ ID NO: 1300 - protein, 9 aa, synthetic construct
  source 1..9: note = Seed: VVGAVGVGK (KRAS G12V)
VGGAVGVGL

SEQ ID NO: 1301 - protein, 9 aa, synthetic construct
  source 1..9: note = Seed: AGDVGKSAL (KRAS G13D)
AFDVGKSAK

SEQ ID NO: 1302 - protein, 9 aa, synthetic construct
  source 1..9: note = Seed: VVGADGVGK (KRAS G12D)
VVGADGVGL

SEQ ID NO: 1303 - protein, 9 aa, synthetic construct
  source 1..9: note = Seed: GARGVGKSA (KRAS G12R)
GERGVGKSM

SEQ ID NO: 1304 - protein, 9 aa, synthetic construct
  source 1..9: note = Seed: VVGAGDVGK (KRAS G13D)
VYGAGDVGR

SEQ ID NO: 1305 - protein, 9 aa, synthetic construct
  source 1..9: note = Seed: LVVVGACGV (KRAS G12C)
LAVVGACGL

SEQ ID NO: 1306 - protein, 9 aa, synthetic construct
  source 1..9: note = Seed: DVGKSALTI (KRAS G13D)
DMGKSALTL

SEQ ID NO: 1307 - protein, 9 aa, synthetic construct
  source 1..9: note = Seed: VVGADGVGK (KRAS G12D)
VYGADGVGR

SEQ ID NO: 1308 - protein, 9 aa, synthetic construct
  source 1..9: note = Seed: GARGVGKSA (KRAS G12R)
GARGVGKSP

SEQ ID NO: 1309 - protein, 9 aa, synthetic construct
  source 1..9: note = Seed: GADGVGKSA (KRAS G12D)
GHDGVGKSY

SEQ ID NO: 1310 - protein, 9 aa, synthetic construct
  source 1..9: note = Seed: VVGADGVGK (KRAS G12D)
VCGADGVGK

SEQ ID NO: 1311 - protein, 9 aa, synthetic construct
  source 1..9: note = Seed: GADGVGKSA (KRAS G12D)
GEDGVGKSV

SEQ ID NO: 1312 - protein, 9 aa, synthetic construct
  source 1..9: note = Seed: GAGDVGKSA (KRAS G13D)
GEGDVGKSR

SEQ ID NO: 1313 - protein, 9 aa, synthetic construct
  source 1..9: note = Seed: VVGAVGVGK (KRAS G12V)
VPGAVGVGY

SEQ ID NO: 1314 - protein, 9 aa, synthetic construct
  source 1..9: note = Seed: VVGADGVGK (KRAS G12D)
VCGADGVGR

SEQ ID NO: 1315 - protein, 9 aa, synthetic construct
  source 1..9: note = Seed: GAGDVGKSA (KRAS G13D)
GQGDVGKSH

SEQ ID NO: 1316 - protein, 9 aa, synthetic construct
  source 1..9: note = Seed: VVGAGDVGK (KRAS G13D)
VCGAGDVGK

SEQ ID NO: 1317 - protein, 9 aa, synthetic construct
  source 1..9: note = Seed: GARGVGKSA (KRAS G12R)
GLRGVGKSH

SEQ ID NO: 1318 - protein, 9 aa, synthetic construct
  source 1..9: note = Seed: GARGVGKSA (KRAS G12R)
GMRGVGKSH

SEQ ID NO: 1319 - protein, 9 aa, synthetic construct
  source 1..9: note = Seed: GADGVGKSA (KRAS G12D)
GMDGVGKSA

SEQ ID NO: 1320 - protein, 9 aa, synthetic construct
  source 1..9: note = Seed: VVGADGVGK (KRAS G12D)
VAGADGVGI

SEQ ID NO: 1321 - protein, 9 aa, synthetic construct
  source 1..9: note = Seed: DVGKSALTI (KRAS G13D)
DEGKSALTI

SEQ ID NO: 1322 - protein, 9 aa, synthetic construct
  source 1..9: note = Seed: AGDVGKSAL (KRAS G13D)
AVDVGKSAT

SEQ ID NO: 1323 - protein, 9 aa, synthetic construct
  source 1..9: note = Seed: AGDVGKSAL (KRAS G13D)
AADVGKSAS

SEQ ID NO: 1324 - protein, 9 aa, synthetic construct
  source 1..9: note = Seed: GARGVGKSA (KRAS G12R)
GARGVGKSQ

SEQ ID NO: 1325 - protein, 9 aa, synthetic construct
  source 1..9: note = Seed: LVVVGACGV (KRAS G12C)
LAVVGACGI

SEQ ID NO: 1326 - protein, 9 aa, synthetic construct
  source 1..9: note = Seed: VVGACGVGK (KRAS G12C)
VIGACGVGM

SEQ ID NO: 1327 - protein, 9 aa, synthetic construct
  source 1..9: note = Seed: GADGVGKSA (KRAS G12D)
GWDGVGKSY

SEQ ID NO: 1328 - protein, 9 aa, synthetic construct
  source 1..9: note = Seed: VVGADGVGK (KRAS G12D)
VHGADGVGK

SEQ ID NO: 1329 - protein, 9 aa, synthetic construct
  source 1..9: note = Seed: GARGVGKSA (KRAS G12R)
GQRGVGKSH

SEQ ID NO: 1330 - protein, 9 aa, synthetic construct
  source 1..9: note = Seed: GAVGVGKSA (KRAS G12V)
GAVGVGKSP

SEQ ID NO: 1331 - protein, 9 aa, synthetic construct
  source 1..9: note = Seed: VVGADGVGK (KRAS G12D)
VAGADGVGV

SEQ ID NO: 1332 - protein, 9 aa, synthetic construct
  source 1..9: note = Seed: VVGAGDVGK (KRAS G13D)
VQGAGDVGY

SEQ ID NO: 1333 - protein, 9 aa, synthetic construct
  source 1..9: note = Seed: VVGAVGVGK (KRAS G12V)
VEGAVGVGY

SEQ ID NO: 1334 - protein, 9 aa, synthetic construct
  source 1..9: note = Seed: VVGACGVGK (KRAS G12C)
VDGACGVGY

SEQ ID NO: 1335 - protein, 9 aa, synthetic construct
  source 1..9: note = Seed: VVGAVGVGK (KRAS G12V)
VFGAVGVGM

SEQ ID NO: 1336 - protein, 9 aa, synthetic construct
  source 1..9: note = Seed: LVVVGAVGV (KRAS G12V)
LAVVGAVGI

SEQ ID NO: 1337 - protein, 9 aa, synthetic construct
  source 1..9: note = Seed: GADGVGKSA (KRAS G12D)
GFDGVGKSA

SEQ ID NO: 1338 - protein, 9 aa, synthetic construct
  source 1..9: note = Seed: GAGDVGKSA (KRAS G13D)
GTGDVGKSW

SEQ ID NO: 1339 - protein, 9 aa, synthetic construct
  source 1..9: note = Seed: VVGADGVGK (KRAS G12D)
VWGADGVGK

SEQ ID NO: 1340 - protein, 9 aa, synthetic construct
  source 1..9: note = Seed: GADGVGKSA (KRAS G12D)
GGDGVGKSW

SEQ ID NO: 1341 - protein, 9 aa, synthetic construct
  source 1..9: note = Seed: VVGARGVGK (KRAS G12R)
VEGARGVGK

SEQ ID NO: 1342 - protein, 9 aa, synthetic construct
  source 1..9: note = Seed: VVGAVGVGK (KRAS G12V)
VTGAVGVGV

SEQ ID NO: 1343 - protein, 9 aa, synthetic construct
  source 1..9: note = Seed: GAGDVGKSA (KRAS G13D)
GEGDVGKSM

SEQ ID NO: 1344 - protein, 9 aa, synthetic construct
  source 1..9: note = Seed: DVGKSALTI (KRAS G13D)
DLGKSALTF

SEQ ID NO: 1345 - protein, 9 aa, synthetic construct
  source 1..9: note = Seed: GADGVGKSA (KRAS G12D)
GPDGVGKSG

SEQ ID NO: 1346 - protein, 9 aa, synthetic construct
  source 1..9: note = Seed: GAVGVGKSA (KRAS G12V)
GWVGVGKSK

SEQ ID NO: 1347 - protein, 9 aa, synthetic construct
  source 1..9: note = Seed: GAGDVGKSA (KRAS G13D)
GWGDVGKSK

SEQ ID NO: 1348 - protein, 9 aa, synthetic construct
  source 1..9: note = Seed: VVGAGDVGK (KRAS G13D)
VHGAGDVGK

SEQ ID NO: 1349 - protein, 9 aa, synthetic construct
  source 1..9: note = Seed: VVGAGDVGK (KRAS G13D)
VWGAGDVGK

SEQ ID NO: 1350 - protein, 9 aa, synthetic construct
  source 1..9: note = Seed: VVGAVGVGK (KRAS G12V)
VTGAVGVGI

SEQ ID NO: 1351 - protein, 9 aa, synthetic construct
  source 1..9: note = Seed: VVGARGVGK (KRAS G12R)
VFGARGVGK

SEQ ID NO: 1352 - protein, 9 aa, synthetic construct
  source 1..9: note = Seed: DVGKSALTI (KRAS G13D)
DMGKSALTF

SEQ ID NO: 1353 - protein, 9 aa, synthetic construct
  source 1..9: note = Seed: GACGVGKSA (KRAS G12C)
GMCGVGKSH

SEQ ID NO: 1354 - protein, 9 aa, synthetic construct
  source 1..9: note = Seed: VVGADGVGK (KRAS G12D)
VVGADGVGH

SEQ ID NO: 1355 - protein, 9 aa, synthetic construct
  source 1..9: note = Seed: GAVGVGKSA (KRAS G12V)
GYVGVGKSY

SEQ ID NO: 1356 - protein, 9 aa, synthetic construct
  source 1..9: note = Seed: LVVVGARGV (KRAS G12R)
LIVVGARGL

SEQ ID NO: 1357 - protein, 9 aa, synthetic construct
  source 1..9: note = Seed: GARGVGKSA (KRAS G12R)
GYRGVGKSY

SEQ ID NO: 1358 - protein, 9 aa, synthetic construct
  source 1..9: note = Seed: GAVGVGKSA (KRAS G12V)
GDVGVGKSF

SEQ ID NO: 1359 - protein, 9 aa, synthetic construct
  source 1..9: note = Seed: GACGVGKSA (KRAS G12C)
GVCGVGKSW

SEQ ID NO: 1360 - protein, 9 aa, synthetic construct
  source 1..9: note = Seed: AGDVGKSAL (KRAS G13D)
AEDVGKSAR

SEQ ID NO: 1361 - protein, 9 aa, synthetic construct
  source 1..9: note = Seed: GADGVGKSA (KRAS G12D)
GIDGVGKSH

SEQ ID NO: 1362 - protein, 9 aa, synthetic construct
  source 1..9: note = Seed: VVGACGVGK (KRAS G12C)
VSGACGVGH

SEQ ID NO: 1363 - protein, 9 aa, synthetic construct
  source 1..9: note = Seed: GAVGVGKSA (KRAS G12V)
GEVGVGKSM

SEQ ID NO: 1364 - protein, 9 aa, synthetic construct
  source 1..9: note = Seed: VVGAGDVGK (KRAS G13D)
VCGAGDVGR

SEQ ID NO: 1365 - protein, 9 aa, synthetic construct
  source 1..9: note = Seed: GACGVGKSA (KRAS G12C)
GQCGVGKSH

SEQ ID NO: 1366 - protein, 9 aa, synthetic construct
  source 1..9: note = Seed: AGDVGKSAL (KRAS G13D)
AFDVGKSAR

SEQ ID NO: 1367 - protein, 9 aa, synthetic construct
  source 1..9: note = Seed: VVGARGVGK (KRAS G12R)
VDGARGVGY

SEQ ID NO: 1368 - protein, 9 aa, synthetic construct
  source 1..9: note = Seed: GARGVGKSA (KRAS G12R)
GMRGVGKSA

SEQ ID NO: 1369 - protein, 9 aa, synthetic construct
  source 1..9: note = Seed: AGDVGKSAL (KRAS G13D)
ADDVGKSAC

SEQ ID NO: 1370 - protein, 9 aa, synthetic construct
  source 1..9: note = Seed: VVGAVGVGK (KRAS G12V)
VQGAVGVGM

SEQ ID NO: 1371 - protein, 9 aa, synthetic construct
  source 1..9: note = Seed: LVVVGAVGV (KRAS G12V)
LIVVGAVGM

SEQ ID NO: 1372 - protein, 9 aa, synthetic construct
  source 1..9: note = Seed: GAGDVGKSA (KRAS G13D)
GPGDVGKSR

SEQ ID NO: 1373 - protein, 9 aa, synthetic construct
  source 1..9: note = Seed: VVGACGVGK (KRAS G12C)
VTGACGVGH

SEQ ID NO: 1374 - protein, 9 aa, synthetic construct
  source 1..9: note = Seed: VVGARGVGK (KRAS G12R)
VMGARGVGF

SEQ ID NO: 1375 - protein, 9 aa, synthetic construct
  source 1..9: note = Seed: DVGKSALTI (KRAS G13D)
DIGKSALTM

SEQ ID NO: 1376 - protein, 9 aa, synthetic construct
  source 1..9: note = Seed: VVGARGVGK (KRAS G12R)
VNGARGVGK

SEQ ID NO: 1377 - protein, 9 aa, synthetic construct
  source 1..9: note = Seed: GADGVGKSA (KRAS G12D)
GWDGVGKSC

SEQ ID NO: 1378 - protein, 9 aa, synthetic construct
  source 1..9: note = Seed: GADGVGKSA (KRAS G12D)
GQDGVGKSW

SEQ ID NO: 1379 - protein, 9 aa, synthetic construct
  source 1..9: note = Seed: VVGACGVGK (KRAS G12C)
VMGACGVGF

SEQ ID NO: 1380 - protein, 9 aa, synthetic construct
  source 1..9: note = Seed: GARGVGKSA (KRAS G12R)
GLRGVGKSA

SEQ ID NO: 1381 - protein, 9 aa, synthetic construct
  source 1..9: note = Seed: GAGDVGKSA (KRAS G13D)
GAGDVGKSQ

SEQ ID NO: 1382 - protein, 9 aa, synthetic construct
  source 1..9: note = Seed: VVGAVGVGK (KRAS G12V)
VHGAVGVGR

SEQ ID NO: 1383 - protein, 9 aa, synthetic construct
  source 1..9: note = Seed: AGDVGKSAL (KRAS G13D)
AYDVGKSAK

SEQ ID NO: 1384 - protein, 9 aa, synthetic construct
  source 1..9: note = Seed: GARGVGKSA (KRAS G12R)
GPRGVGKSN

SEQ ID NO: 1385 - protein, 9 aa, synthetic construct
  source 1..9: note = Seed: GAVGVGKSA (KRAS G12V)
GIVGVGKSW

SEQ ID NO: 1386 - protein, 9 aa, synthetic construct
  source 1..9: note = Seed: GARGVGKSA (KRAS G12R)
GFRGVGKSF

SEQ ID NO: 1387 - protein, 9 aa, synthetic construct
  source 1..9: note = Seed: GADGVGKSA (KRAS G12D)
GCDGVGKSA

SEQ ID NO: 1388 - protein, 9 aa, synthetic construct
  source 1..9: note = Seed: AGDVGKSAL (KRAS G13D)
ADDVGKSAA

SEQ ID NO: 1389 - protein, 9 aa, synthetic construct
  source 1..9: note = Seed: GAVGVGKSA (KRAS G12V)
GQVGVGKSW

SEQ ID NO: 1390 - protein, 9 aa, synthetic construct
  source 1..9: note = Seed: GAVGVGKSA (KRAS G12V)
GGVGVGKSI

SEQ ID NO: 1391 - protein, 9 aa, synthetic construct
  source 1..9: note = Seed: GAGDVGKSA (KRAS G13D)
GIGDVGKSW

SEQ ID NO: 1392 - protein, 9 aa, synthetic construct
  source 1..9: note = Seed: LVVVGAGDV (KRAS G13D)
LMVVGAGDL

SEQ ID NO: 1393 - protein, 9 aa, synthetic construct
  source 1..9: note = Seed: VVGADGVGK (KRAS G12D)
VGGADGVGF

SEQ ID NO: 1394 - protein, 9 aa, synthetic construct
  source 1..9: note = Seed: GACGVGKSA (KRAS G12C)
GLCGVGKSH

SEQ ID NO: 1395 - protein, 9 aa, synthetic construct
  source 1..9: note = Seed: LVVVGARGV (KRAS G12R)
LQVVGARGL

SEQ ID NO: 1396 - protein, 9 aa, synthetic construct
  source 1..9: note = Seed: VVGAGDVGK (KRAS G13D)
VAGAGDVGI

SEQ ID NO: 1397 - protein, 9 aa, synthetic construct
  source 1..9: note = Seed: GACGVGKSA (KRAS G12C)
GTCGVGKSW

SEQ ID NO: 1398 - protein, 9 aa, synthetic construct
  source 1..9: note = Seed: LVVVGADGV (KRAS G12D)
LVVVGADGA

SEQ ID NO: 1399 - protein, 9 aa, synthetic construct
  source 1..9: note = Seed: GACGVGKSA (KRAS G12C)
GTCGVGKSA

SEQ ID NO: 1400 - protein, 9 aa, synthetic construct
  source 1..9: note = Seed: GADGVGKSA (KRAS G12D)
GHDGVGKSK

SEQ ID NO: 1401 - protein, 9 aa, synthetic construct
  source 1..9: note = Seed: GAVGVGKSA (KRAS G12V)
GNVGVGKSL

SEQ ID NO: 1402 - protein, 9 aa, synthetic construct
  source 1..9: note = Seed: VVGARGVGK (KRAS G12R)
VFGARGVGM

SEQ ID NO: 1403 - protein, 9 aa, synthetic construct
  source 1..9: note = Seed: GARGVGKSA (KRAS G12R)
GNRGVGKSL

SEQ ID NO: 1404 - protein, 9 aa, synthetic construct
  source 1..9: note = Seed: GACGVGKSA (KRAS G12C)
GPCGVGKSW

SEQ ID NO: 1405 - protein, 9 aa, synthetic construct
  source 1..9: note = Seed: GAGDVGKSA (KRAS G13D)
GTGDVGKSA

SEQ ID NO: 1406 - protein, 9 aa, synthetic construct
  source 1..9: note = Seed: LVVVGAVGV (KRAS G12V)
LSVVGAVGL

SEQ ID NO: 1407 - protein, 9 aa, synthetic construct
  source 1..9: note = Seed: DVGKSALTI (KRAS G13D)
DMGKSALTY

SEQ ID NO: 1408 - protein, 9 aa, synthetic construct
  source 1..9: note = Seed: GACGVGKSA (KRAS G12C)
GECGVGKSM

SEQ ID NO: 1409 - protein, 9 aa, synthetic construct
  source 1..9: note = Seed: GADGVGKSA (KRAS G12D)
GADGVGKSP

SEQ ID NO: 1410 - protein, 9 aa, synthetic construct
  source 1..9: note = Seed: VVGAVGVGK (KRAS G12V)
VGGAVGVGH

SEQ ID NO: 1411 - protein, 9 aa, synthetic construct
  source 1..9: note = Seed: GARGVGKSA (KRAS G12R)
GARGVGKSS

SEQ ID NO: 1412 - protein, 9 aa, synthetic construct
  source 1..9: note = Seed: GACGVGKSA (KRAS G12C)
GPCGVGKSK

SEQ ID NO: 1413 - protein, 9 aa, synthetic construct
  source 1..9: note = Seed: GACGVGKSA (KRAS G12C)
GPCGVGKSR

SEQ ID NO: 1414 - protein, 9 aa, synthetic construct
  source 1..9: note = Seed: VVGADGVGK (KRAS G12D)
VDGADGVGY

SEQ ID NO: 1415 - protein, 9 aa, synthetic construct
  source 1..9: note = Seed: VVGARGVGK (KRAS G12R)
VMGARGVGM

SEQ ID NO: 1416 - protein, 9 aa, synthetic construct
  source 1..9: note = Seed: GAVGVGKSA (KRAS G12V)
GPVGVGKSQ

SEQ ID NO: 1417 - protein, 9 aa, synthetic construct
  source 1..9: note = Seed: VVGARGVGK (KRAS G12R)
VMGARGVGL

SEQ ID NO: 1418 - protein, 9 aa, synthetic construct
  source 1..9: note = Seed: VVGAVGVGK (KRAS G12V)
VFGAVGVGL

SEQ ID NO: 1419 - protein, 9 aa, synthetic construct
  source 1..9: note = Seed: VVGADGVGK (KRAS G12D)
VWGADGVGR

SEQ ID NO: 1420 - protein, 9 aa, synthetic construct
  source 1..9: note = Seed: DVGKSALTI (KRAS G13D)
DNGKSALTV

SEQ ID NO: 1421 - protein, 9 aa, synthetic construct
  source 1..9: note = Seed: VVGACGVGK (KRAS G12C)
VNGACGVGY

SEQ ID NO: 1422 - protein, 9 aa, synthetic construct
  source 1..9: note = Seed: VVGAGDVGK (KRAS G13D)
VVGAGDVGL

SEQ ID NO: 1423 - protein, 9 aa, synthetic construct
  source 1..9: note = Seed: GARGVGKSA (KRAS G12R)
GCRGVGKSL

SEQ ID NO: 1424 - protein, 9 aa, synthetic construct
  source 1..9: note = Seed: VVGARGVGK (KRAS G12R)
VLGARGVGF

SEQ ID NO: 1425 - protein, 9 aa, synthetic construct
  source 1..9: note = Seed: GARGVGKSA (KRAS G12R)
GIRGVGKSW

SEQ ID NO: 1426 - protein, 9 aa, synthetic construct
  source 1..9: note = Seed: VVGACGVGK (KRAS G12C)
VCGACGVGK

SEQ ID NO: 1427 - protein, 9 aa, synthetic construct
  source 1..9: note = Seed: VVGAVGVGK (KRAS G12V)
VMGAVGVGL

SEQ ID NO: 1428 - protein, 9 aa, synthetic construct
  source 1..9: note = Seed: GARGVGKSA (KRAS G12R)
GGRGVGKSV

SEQ ID NO: 1429 - protein, 9 aa, synthetic construct
  source 1..9: note = Seed: AGDVGKSAL (KRAS G13D)
AADVGKSAP

SEQ ID NO: 1430 - protein, 9 aa, synthetic construct
  source 1..9: note = Seed: VVGAGDVGK (KRAS G13D)
VWGAGDVGR

SEQ ID NO: 1431 - protein, 9 aa, synthetic construct
  source 1..9: note = Seed: VVGAVGVGK (KRAS G12V)
VLGAVGVGL

SEQ ID NO: 1432 - protein, 9 aa, synthetic construct
  source 1..9: note = Seed: VVGARGVGK (KRAS G12R)
VTGARGVGH

SEQ ID NO: 1433 - protein, 9 aa, synthetic construct
  source 1..9: note = Seed: VVGAVGVGK (KRAS G12V)
VMGAVGVGM

SEQ ID NO: 1434 - protein, 9 aa, synthetic construct
  source 1..9: note = Seed: GAVGVGKSA (KRAS G12V)
GHVGVGKSK

SEQ ID NO: 1435 - protein, 9 aa, synthetic construct
  source 1..9: note = Seed: VVGAVGVGK (KRAS G12V)
VAGAVGVGQ

SEQ ID NO: 1436 - protein, 9 aa, synthetic construct
  source 1..9: note = Seed: GADGVGKSA (KRAS G12D)
GRDGVGKSW

SEQ ID NO: 1437 - protein, 9 aa, synthetic construct
  source 1..9: note = Seed: GAGDVGKSA (KRAS G13D)
GIGDVGKSA

SEQ ID NO: 1438 - protein, 9 aa, synthetic construct
  source 1..9: note = Seed: DVGKSALTI (KRAS G13D)
DCGKSALTV

SEQ ID NO: 1439 - protein, 9 aa, synthetic construct
  source 1..9: note = Seed: VVGACGVGK (KRAS G12C)
VVGACGVGH

SEQ ID NO: 1440 - protein, 9 aa, synthetic construct
  source 1..9: note = Seed: DVGKSALTI (KRAS G13D)
DLGKSALTY

SEQ ID NO: 1441 - protein, 9 aa, synthetic construct
  source 1..9: note = Seed: GACGVGKSA (KRAS G12C)
GYCGVGKSK

SEQ ID NO: 1442 - protein, 9 aa, synthetic construct
  source 1..9: note = Seed: AGDVGKSAL (KRAS G13D)
ACDVGKSAC

SEQ ID NO: 1443 - protein, 9 aa, synthetic construct
  source 1..9: note = Seed: GADGVGKSA (KRAS G12D)
GDDGVGKSY

SEQ ID NO: 1444 - protein, 9 aa, synthetic construct
  source 1..9: note = Seed: GAVGVGKSA (KRAS G12V)
GPVGVGKSE

SEQ ID NO: 1445 - protein, 9 aa, synthetic construct
  source 1..9: note = Seed: VVGADGVGK (KRAS G12D)
VIGADGVGM

SEQ ID NO: 1446 - protein, 9 aa, synthetic construct
  source 1..9: note = Seed: AGDVGKSAL (KRAS G13D)
ACDVGKSAA

SEQ ID NO: 1447 - protein, 9 aa, synthetic construct
  source 1..9: note = Seed: VVGADGVGK (KRAS G12D)
VNGADGVGY

SEQ ID NO: 1448 - protein, 9 aa, synthetic construct
  source 1..9: note = Seed: GAVGVGKSA (KRAS G12V)
GFVGVGKSM

SEQ ID NO: 1449 - protein, 9 aa, synthetic construct
  source 1..9: note = Seed: VVGARGVGK (KRAS G12R)
VNGARGVGR

SEQ ID NO: 1450 - protein, 9 aa, synthetic construct
  source 1..9: note = Seed: VVGARGVGK (KRAS G12R)
VLGARGVGM

SEQ ID NO: 1451 - protein, 9 aa, synthetic construct
  source 1..9: note = Seed: GAGDVGKSA (KRAS G13D)
GQGDVGKSW

SEQ ID NO: 1452 - protein, 9 aa, synthetic construct
  source 1..9: note = Seed: LVVVGADGV (KRAS G12D)
LIVVGADGA

SEQ ID NO: 1453 - protein, 9 aa, synthetic construct
  source 1..9: note = Seed: GAVGVGKSA (KRAS G12V)
GIVGVGKSA

SEQ ID NO: 1454 - protein, 9 aa, synthetic construct
  source 1..9: note = Seed: VVGACGVGK (KRAS G12C)
VHGACGVGK

SEQ ID NO: 1455 - protein, 9 aa, synthetic construct
  source 1..9: note = Seed: LVVVGAGDV (KRAS G13D)
LIVVGAGDV

SEQ ID NO: 1456 - protein, 9 aa, synthetic construct
  source 1..9: note = Seed: VVGAGDVGK (KRAS G13D)
VAGAGDVGV

SEQ ID NO: 1457 - protein, 9 aa, synthetic construct
  source 1..9: note = Seed: GACGVGKSA (KRAS G12C)
GDCGVGKSF

SEQ ID NO: 1458 - protein, 9 aa, synthetic construct
  source 1..9: note = Seed: GACGVGKSA (KRAS G12C)
GFCGVGKSR

SEQ ID NO: 1459 - protein, 9 aa, synthetic construct
  source 1..9: note = Seed: GACGVGKSA (KRAS G12C)
GECGVGKSR

SEQ ID NO: 1460 - protein, 9 aa, synthetic construct
  source 1..9: note = Seed: GARGVGKSA (KRAS G12R)
GTRGVGKSW

SEQ ID NO: 1461 - protein, 9 aa, synthetic construct
  source 1..9: note = Seed: GAGDVGKSA (KRAS G13D)
GDGDVGKSF

SEQ ID NO: 1462 - protein, 9 aa, synthetic construct
  source 1..9: note = Seed: GAVGVGKSA (KRAS G12V)
GNVGVGKSH

SEQ ID NO: 1463 - protein, 9 aa, synthetic construct
  source 1..9: note = Seed: GACGVGKSA (KRAS G12C)
GICGVGKSA

SEQ ID NO: 1464 - protein, 9 aa, synthetic construct
  source 1..9: note = Seed: VVGACGVGK (KRAS G12C)
VWGACGVGK

SEQ ID NO: 1465 - protein, 9 aa, synthetic construct
  source 1..9: note = Seed: GACGVGKSA (KRAS G12C)
GCCGVGKSM

SEQ ID NO: 1466 - protein, 9 aa, synthetic construct
  source 1..9: note = Seed: LVVVGAGDV (KRAS G13D)
LMVVGAGDI

SEQ ID NO: 1467 - protein, 9 aa, synthetic construct
  source 1..9: note = Seed: VVGACGVGK (KRAS G12C)
VAGACGVGW

SEQ ID NO: 1468 - protein, 9 aa, synthetic construct
  source 1..9: note = Seed: GAGDVGKSA (KRAS G13D)
GYGDVGKSR

SEQ ID NO: 1469 - protein, 9 aa, synthetic construct
  source 1..9: note = Seed: LVVVGARGV (KRAS G12R)
LQVVGARGI

SEQ ID NO: 1470 - protein, 9 aa, synthetic construct
  source 1..9: note = Seed: LVVVGAVGV (KRAS G12V)
LLVVGAVGT

SEQ ID NO: 1471 - protein, 9 aa, synthetic construct
  source 1..9: note = Seed: VVGAGDVGK (KRAS G13D)
VSGAGDVGH

SEQ ID NO: 1472 - protein, 9 aa, synthetic construct
  source 1..9: note = Seed: AGDVGKSAL (KRAS G13D)
AIDVGKSAH

SEQ ID NO: 1473 - protein, 9 aa, synthetic construct
  source 1..9: note = Seed: VVGARGVGK (KRAS G12R)
VSGARGVGW

SEQ ID NO: 1474 - protein, 9 aa, synthetic construct
  source 1..9: note = Seed: GACGVGKSA (KRAS G12C)
GACGVGKSQ

SEQ ID NO: 1475 - protein, 9 aa, synthetic construct
  source 1..9: note = Seed: VVGACGVGK (KRAS G12C)
VQGACGVGF

SEQ ID NO: 1476 - protein, 9 aa, synthetic construct
  source 1..9: note = Seed: VVGACGVGK (KRAS G12C)
VCGACGVGY

SEQ ID NO: 1477 - protein, 9 aa, synthetic construct
  source 1..9: note = Seed: VVGADGVGK (KRAS G12D)
VCGADGVGY

SEQ ID NO: 1478 - protein, 9 aa, synthetic construct
  source 1..9: note = Seed: LVVVGAGDV (KRAS G13D)
LLVVGAGDL

SEQ ID NO: 1479 - protein, 9 aa, synthetic construct
  source 1..9: note = Seed: AGDVGKSAL (KRAS G13D)
ACDVGKSAW

SEQ ID NO: 1480 - protein, 9 aa, synthetic construct
  source 1..9: note = Seed: LVVVGADGV (KRAS G12D)
LAVVGADGL

SEQ ID NO: 1481 - protein, 9 aa, synthetic construct
  source 1..9: note = Seed: VVGAVGVGK (KRAS G12V)
VCGAVGVGF

SEQ ID NO: 1482 - protein, 9 aa, synthetic construct
  source 1..9: note = Seed: VVGADGVGK (KRAS G12D)
VHGADGVGR

SEQ ID NO: 1483 - protein, 9 aa, synthetic construct
  source 1..9: note = Seed: GACGVGKSA (KRAS G12C)
GCCGVGKSK

SEQ ID NO: 1484 - protein, 9 aa, synthetic construct
  source 1..9: note = Seed: AGDVGKSAL (KRAS G13D)
AWDVGKSAT

SEQ ID NO: 1485 - protein, 9 aa, synthetic construct
  source 1..9: note = Seed: LVVVGACGV (KRAS G12C)
LSVVGACGI

SEQ ID NO: 1486 - protein, 9 aa, synthetic construct
  source 1..9: note = Seed: GACGVGKSA (KRAS G12C)
GCCGVGKSR

SEQ ID NO: 1487 - protein, 9 aa, synthetic construct
  source 1..9: note = Seed: LVVVGAVGV (KRAS G12V)
LSVVGAVGI

SEQ ID NO: 1488 - protein, 9 aa, synthetic construct
  source 1..9: note = Seed: AGDVGKSAL (KRAS G13D)
AYDVGKSAR

SEQ ID NO: 1489 - protein, 9 aa, synthetic construct
  source 1..9: note = Seed: VVGARGVGK (KRAS G12R)
VLGARGVGL

SEQ ID NO: 1490 - protein, 9 aa, synthetic construct
  source 1..9: note = Seed: LVVVGAVGV (KRAS G12V)
LQVVGAVGM

SEQ ID NO: 1491 - protein, 9 aa, synthetic construct
  source 1..9: note = Seed: LVVVGAVGV (KRAS G12V)
LTVVGAVGM

SEQ ID NO: 1492 - protein, 9 aa, synthetic construct
  source 1..9: note = Seed: VVGADGVGK (KRAS G12D)
VTGADGVGH

SEQ ID NO: 1493 - protein, 9 aa, synthetic construct
  source 1..9: note = Seed: VVGADGVGK (KRAS G12D)
VQGADGVGF

SEQ ID NO: 1494 - protein, 9 aa, synthetic construct
  source 1..9: note = Seed: GADGVGKSA (KRAS G12D)
GYDGVGKST

SEQ ID NO: 1495 - protein, 9 aa, synthetic construct
  source 1..9: note = Seed: VVGACGVGK (KRAS G12C)
VIGACGVGL

SEQ ID NO: 1496 - protein, 9 aa, synthetic construct
  source 1..9: note = Seed: VVGAVGVGK (KRAS G12V)
VLGAVGVGM

SEQ ID NO: 1497 - protein, 9 aa, synthetic construct
  source 1..9: note = Seed: LVVVGACGV (KRAS G12C)
LSVVGACGL

SEQ ID NO: 1498 - protein, 9 aa, synthetic construct
  source 1..9: note = Seed: VVGAGDVGK (KRAS G13D)
VNGAGDVGY

SEQ ID NO: 1499 - protein, 9 aa, synthetic construct
  source 1..9: note = Seed: VVGADGVGK (KRAS G12D)
VMGADGVGF

SEQ ID NO: 1500 - protein, 9 aa, synthetic construct
  source 1..9: note = Seed: GAGDVGKSA (KRAS G13D)
GWGDVGKSR

SEQ ID NO: 1501 - protein, 9 aa, synthetic construct
  source 1..9: note = Seed: AGDVGKSAL (KRAS G13D)
AWDVGKSAK

SEQ ID NO: 1502 - protein, 9 aa, synthetic construct
  source 1..9: note = Seed: GAGDVGKSA (KRAS G13D)
GNGDVGKSL

SEQ ID NO: 1503 - protein, 9 aa, synthetic construct
  source 1..9: note = Seed: VVGAVGVGK (KRAS G12V)
VSGAVGVGW

SEQ ID NO: 1504 - protein, 9 aa, synthetic construct
  source 1..9: note = Seed: GARGVGKSA (KRAS G12R)
GQRGVGKSV

SEQ ID NO: 1505 - protein, 9 aa, synthetic construct
  source 1..9: note = Seed: VVGARGVGK (KRAS G12R)
VFGARGVGL

SEQ ID NO: 1506 - protein, 9 aa, synthetic construct
  source 1..9: note = Seed: VVGACGVGK (KRAS G12C)
VGGACGVGM

SEQ ID NO: 1507 - protein, 9 aa, synthetic construct
  source 1..9: note = Seed: DVGKSALTI (KRAS G13D)
DLGKSALTL

SEQ ID NO: 1508 - protein, 9 aa, synthetic construct
  source 1..9: note = Seed: GARGVGKSA (KRAS G12R)
GVRGVGKSC

SEQ ID NO: 1509 - protein, 9 aa, synthetic construct
  source 1..9: note = Seed: LVVVGACGV (KRAS G12C)
LQVVGACGM

SEQ ID NO: 1510 - protein, 9 aa, synthetic construct
  source 1..9: note = Seed: VVGARGVGK (KRAS G12R)
VYGARGVGY

SEQ ID NO: 1511 - protein, 9 aa, synthetic construct
  source 1..9: note = Seed: VVGADGVGK (KRAS G12D)
VYGADGVGY

SEQ ID NO: 1512 - protein, 9 aa, synthetic construct
  source 1..9: note = Seed: GACGVGKSA (KRAS G12C)
GGCGVGKSV

SEQ ID NO: 1513 - protein, 9 aa, synthetic construct
  source 1..9: note = Seed: VVGARGVGK (KRAS G12R)
VEGARGVGR

SEQ ID NO: 1514 - protein, 9 aa, synthetic construct
  source 1..9: note = Seed: VVGACGVGK (KRAS G12C)
VCGACGVGR

SEQ ID NO: 1515 - protein, 9 aa, synthetic construct
  source 1..9: note = Seed: GARGVGKSA (KRAS G12R)
GGRGVGKSI

SEQ ID NO: 1516 - protein, 9 aa, synthetic construct
  source 1..9: note = Seed: GACGVGKSA (KRAS G12C)
GYCGVGKSY

SEQ ID NO: 1517 - protein, 9 aa, synthetic construct
  source 1..9: note = Seed: AGDVGKSAL (KRAS G13D)
APDVGKSAK

SEQ ID NO: 1518 - protein, 9 aa, synthetic construct
  source 1..9: note = Seed: DVGKSALTI (KRAS G13D)
DEGKSALTL

SEQ ID NO: 1519 - protein, 9 aa, synthetic construct
  source 1..9: note = Seed: GARGVGKSA (KRAS G12R)
GERGVGKSL

SEQ ID NO: 1520 - protein, 9 aa, synthetic construct
  source 1..9: note = Seed: GADGVGKSA (KRAS G12D)
GCDGVGKSW

SEQ ID NO: 1521 - protein, 9 aa, synthetic construct
  source 1..9: note = Seed: GAGDVGKSA (KRAS G13D)
GWGDVGKSY

SEQ ID NO: 1522 - protein, 9 aa, synthetic construct
  source 1..9: note = Seed: AGDVGKSAL (KRAS G13D)
ACDVGKSAK

SEQ ID NO: 1523 - protein, 321 aa, synthetic construct
MRVTAPRTLILLLSGALALTETWAGSGGSGGGGSGGGADGVGKSMGGSGGGGSGGLMVVGADGVGGSGGGGSGGGAVGVGKSLGGSGGGGSGGLMVVGAVGVGGSGGGGSGGVTGARGVGKGGSGGGGSGGEYKFVVLGTVGHGKSGGSGGGGSGGEYKIVVAGNVGIGKSGGSGGGGSGGEYKFVVFGSDGAGKSGGSGGGGSGGMTEYKFVVSGADGIGKSALTGGSGGGGSGGMTEYKFVVIGNRGVGKSALTGGSLGGGGSGIVGIVAGLAVLAVVVIGAVVATVMCRRKSSGGKGGSYSQAASSDSAQGSDVSLTA